SEQUENCE LISTING (189 sequences)

SEQ ID NO: 1 - protein, 127 aa, Homo sapiens [sequence omitted]

SEQ ID NO: 2 - RNA, 432 nt, Homo sapiens
auguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggag

SEQ ID NO: 3 - RNA, 788 nt, Homo sapiens
aguacacagagagaaaggcuaaaguucucuggaggauguggcugcagagccugcugcucuugggcacuguggccugcagcaucucugcacccgcccgcucgcccagccccagcacgcagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaagcagcacugcccuccaaccccggaaacuuccugugcaacccagauuaucaccuuugaaaguuucaaagagaaccugaaggacuuucugcuugucauccccuuugacugcugggagccaguccaggagugagaccggccagaugaggcuggccaagccggggagcugcucucucaugaaacaagagcuagaaacucaggauggucaucuuggagggaccaaggggugggccacagccauggugggaguggccuggaccugcccugggccacacugacccugauacaggcauggcagaagaaugggaauauuuuauacugacagaaaucaguaauauuuauauauuuauauuuuuaaaauauuuauuuauuuauuuauuuaaguucauauuccauauuuauucaagauguuuuaccguaauaauuauuauuaaaaauaugcuucuacuug

SEQ ID NO: 4 - RNA, 423 nt, Mus sp.
auguggcuucagaaucucuuguuucuuggaaucgucguguacagccugucagccccaacuagaucgccuaucacugugacgcgcccguggaagcacguggaagccaucaaggaggcucugaaucugcucgacgauaugccagugacccugaacgaggaagucgaagugguguccaacgaauuuuccuucaagaaguugaccuguguucagacccggcugaagauuuucgagcagggccucaggggaaacuucaccaaacugaagggugcacugaacaugaccgccagcuacuaccagaccuauugcccuccgacuccggaaacugauugcgagacucaagucaccaccuacgcggacuucaucgacucgcucaagacguuccugacugacauccccuucgagugcaagaagccggggcagaaa

SEQ ID NO: 5 - RNA, 1033 nt, Mus sp.
ggucagacugcccaggcagggugggaaaggccuuuaaagcagcccgcaggugggcugccaguucuuggaagggcuuauuaaugaaaaccccccaagccugacaaccugggggaaggcucacuggccccauguauagcugauaagggccaggagauuccacaacucagguaguucccccgccccccuggaguucuguggucaccauuaaucauuuccucuaacuguguauauaagagcucuuuugcagugagcccaguacucagagagaaaggcuaagguccugaggaggauguggcugcagaauuuacuuuuccugggcauuguggucuacagccucucagcacccacccgcucacccaucacugucacccggccuuggaagcauguagaggccaucaaagaagcccugaaccuccuggaugacaugccugucacguugaaugaagagguagaagucgucucuaacgaguucuccuucaagaagcuaacaugugugcagacccgccugaagauauucgagcagggucuacggggcaauuucaccaaacucaagggcgccuugaacaugacagccagcuacuaccagacauacugccccccaacuccggaaacggacugugaaacacaaguuaccaccuaugcggauuucauagacagccuuaaaaccuuucugacugauauccccuuugaaugcaaaaaaccaggccaaaaaugaggaagcccaggccagcucugaauccagcuucucagacugcugcuuuugugccugcguaaugagccaggaacuuggaauuucugccuuaaagggaccaagagauguggcacagccacaguuggaaggcaguauagcccucugaaaacgcugacucagcuuggacagcggaagacaaacgagagauauuuucuacugauagggaccauuauauuuauuuauauauuuauauuuuuuaaauauuuauuuauuuauuuauuuauuuuugcaacucuauuuauugagaaugucuuaccagaauaauaaauuauuaaaacuuuu

SEQ ID NO: 6 - RNA, 432 nt, Rattus sp.
auguggcugcagaaccugcuguuccugggcaucgugguguacagcuucagcgccccuaccagaagcccuaacccugugaccagaccuuggaagcacguggacgccaucaaggaggcccugagccugcugaacgacaugagagcccuggagaacgagaagaacgaggacguggacaucaucagcaacgaguucagcauccagagaccuaccugcgugcagaccagacugaagcuguacaagcagggccugagaggcaaccugaccaagcugaacggcgcccugaccaugaucgccagccacuaccagaccaacugcccuccuaccccugagaccgacugcgagaucgaggugaccaccuucgaggacuucaucaagaaccugaagggcuuccuguucgacaucccuuucgacugcuggaagccugugcagaag

SEQ ID NO: 7 - RNA, 441 nt, Rattus sp.
auguggcugcagaauuuacuuuuccugggcauuguggucuacaguuucucagcacccacccgcucgcccaacccugucacccggcccuggaagcauguagaugccaucaaagaagcucugagccuccuaaaugacaugcgugcucuggagaacgaaaagaacgaagacguagacaucaucucuaaugaguucuccauccagaggccgacaugugugcagacccgccugaagcuauacaagcagggucuacggggcaaccucaccaaacucaauggcgccuugaccaugauagccagccacuaccagacgaacugcccuccaaccccggaaacugacugugaaauagaagucaccaccuuugaggauuucauaaagaaccuuaaaggcuuucuguuugauaucccuuuugacugcuggaagccgguccagaaaugaggaggc

SEQ ID NO: 8 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 9 - protein, 141 aa, Mus sp.
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 10 - protein, 144 aa, Rattus sp.
MWLQNLLFLGIVVYSFSAPTRSPNPVTRPWKHVDAIKEALSLLNDMRALENEKNEDVDIISNEFSIQRPTCVQTRLKLYKQGLRGNLTKLNGALTMIASHYQTNCPPTPETDCEIEVTTFEDFIKNLKGFLFDIPFDCWKPVQK

SEQ ID NO: 11 - protein, 144 aa, Macaca fascicularis
MWLQGLLLLGTVACSISAPARSPSPGTQPWEHVNAIQEARRLLNLSRDTAAEMNKTVEVVSEMFDLQEPSCLQTRLELYKQGLQGSLTKLKGPLTMMASHYKQHCPPTPETSCATQIITFQSFKENLKDFLLVIPFDCWEPVQE

SEQ ID NO: 12 - RNA, 432 nt, Macaca fascicularis
auguggcugcagggccugcugcugcugggcaccguggccugcagcaucagcgccccugccagaagcccuagcccuggcacccagccuugggagcacgugaacgccauccaggaggccagaagacugcugaaccugagcagagacaccgccgccgagaugaacaagaccguggagguggugagcgagauguucgaccugcaggagccuagcugccugcagaccagacuggagcuguacaagcagggccugcagggcagccugaccaagcugaagggcccucugaccaugauggccagccacuacaagcagcacugcccuccuaccccugagaccagcugcgccacccagaucaucaccuuccagagcuucaaggagaaccugaaggacuuccugcuggugaucccuuucgacugcugggagccugugcaggag

SEQ ID NO: 13-24 - skipped (12 entries)

SEQ ID NO: 25 - DNA, 100 bp, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 120 bp, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27-86 - skipped (60 entries)

SEQ ID NO: 87 - RNA, 9 nt, Unknown
  source: /note=“Description of Unknown: Kozak sequence”
ccrccaugg

SEQ ID NO: 88 - RNA, 92 nt, Unknown
  source: /note=“Description of Unknown: 5′ UTR-018 (Upstream UTR) 5′ UTR sequence”
ucaagcuuuuggacccucguacagaagcuaauacgacucacuauagggaaauaagagagaaaagaagaguaagaagaaauauaagagccacc

SEQ ID NO: 89 - RNA, 47 nt, Unknown
  source: /note=“Description of Unknown: 5′ UTR-002 (Upstream UTR) sequence”
gggagaucagagagaaaagaagaguaagaagaaauauaagagccacc

SEQ ID NO: 90 - RNA, 42 nt, Unknown
  source: /note=“Description of Unknown: 5′ UTR-004 (Upstream UTR) sequence”
gggagacaagcuuggcauuccgguacuguugguaaagccacc

SEQ ID NO: 91 - RNA, 47 nt, Unknown; identical to SEQ ID NO: 89
  source: /note=“Description of Unknown: 5′ UTR-005 (Upstream UTR) sequence”

SEQ ID NO: 92 - RNA, 42 nt, Unknown; identical to SEQ ID NO: 90
  source: /note=“Description of Unknown: 5′ UTR-007 (Upstream UTR) sequence”

SEQ ID NO: 93 - RNA, 47 nt, Unknown
  source: /note=“Description of Unknown: 5′ UTR-008 (Upstream UTR) sequence”
gggaauuaacagagaaaagaagaguaagaagaaauauaagagccacc

SEQ ID NO: 94 - RNA, 47 nt, Unknown
  source: /note=“Description of Unknown: 5′ UTR-009 (Upstream UTR) sequence”
gggaaauuagacagaaaagaagaguaagaagaaauauaagagccacc

SEQ ID NO: 95 - RNA, 47 nt, Unknown
  source: /note=“Description of Unknown: 5′ UTR-010, Upstream sequence”
gggaaauaagagaguaaagaacaguaagaagaaauauaagagccacc

SEQ ID NO: 96 - RNA, 47 nt, Unknown
  source: /note=“Description of Unknown: 5′ UTR-011 (Upstream UTR) sequence”
gggaaaaaagagagaaaagaagacuaagaagaaauauaagagccacc

SEQ ID NO: 97 - RNA, 47 nt, Unknown
  source: /note=“Description of Unknown: 5′ UTR-012 (Upstream UTR) sequence”
gggaaauaagagagaaaagaagaguaagaagauauauaagagccacc

SEQ ID NO: 98 - RNA, 47 nt, Unknown
  source: /note=“Description of Unknown: 5′ UTR-013 (Upstream UTR) sequence”
gggaaauaagagacaaaacaagaguaagaagaaauauaagagccacc

SEQ ID NO: 99 - RNA, 47 nt, Unknown
  source: /note=“Description of Unknown: 5′ UTR-014 (Upstream UTR) sequence”
gggaaauuagagaguaaagaacaguaaguagaauuaaaagagccacc

SEQ ID NO: 100 - RNA, 47 nt, Unknown
  source: /note=“Description of Unknown: 5′ UTR-015 (Upstream UTR) sequence”
gggaaauaagagagaauagaagaguaagaagaaauauaagagccacc

SEQ ID NO: 101 - RNA, 47 nt, Unknown
  source: /note=“Description of Unknown: 5′ UTR-016 (Upstream UTR) sequence”
gggaaauaagagagaaaagaagaguaagaagaaaauuaagagccacc

SEQ ID NO: 102 - RNA, 47 nt, Unknown
  source: /note=“Description of Unknown: 5′ UTR-017 (Upstream UTR) sequence”
gggaaauaagagagaaaagaagaguaagaagaaauuuaagagccacc

SEQ ID NO: 103 - skipped

SEQ ID NO: 104 - RNA, 142 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 105 - RNA, 142 nt, Artificial Sequence
  source: /note=“Description of Artificial Sequence: Synthetic polynucleotide”
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 106 - RNA, 142 nt, Artificial Sequence
  source: /note=“Description of Artificial Sequence: Synthetic polynucleotide”
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 107 - RNA, 142 nt, Artificial Sequence
  source: /note=“Description of Artificial Sequence: Synthetic polynucleotide”
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 108 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 109 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 110 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 111 - RNA, 164 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 112 - RNA, 164 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 113 - skipped

SEQ ID NO: 114 - RNA, 87 nt, Unknown
  source: /note=“Description of Unknown: miR-142 sequence”
gacagugcagucacccauaaaguagaaagcacuacuaacagcacuggaggguguaguguuuccuacuuuauggaugaguguacugug

SEQ ID NO: 115 - RNA, 23 nt, Unknown
  source: /note=“Description of Unknown: miR-142-3p sequence”
uguaguguuuccuacuuuaugga

SEQ ID NO: 116 - RNA, 23 nt, Unknown
  source: /note=“Description of Unknown: miR 142-3p binding site sequence”
uccauaaaguaggaaacacuaca

SEQ ID NO: 117 - RNA, 21 nt, Unknown
  source: /note=“Description of Unknown: miR-142-5p sequence”
cauaaaguagaaagcacuacu

SEQ ID NO: 118 - RNA, 21 nt, Unknown
  source: /note=“Description of Unknown: miR-142-5p binding site sequence”
aguagugcuuucuacuuuaug

SEQ ID NO: 119 - RNA, 85 nt, Unknown
  source: /note=“Description of Unknown: miR-126 sequence”
cgcuggcgacgggacauuauuacuuuugguacgcgcugugacacuucaaacucguaccgugaguaauaaugcgccguccacggca

SEQ ID NO: 120 - RNA, 22 nt, Unknown
  source: /note=“Description of Unknown: miR-126-3p sequence”
ucguaccgugaguaauaaugcg

SEQ ID NO: 121 - RNA, 22 nt, Unknown
  source: /note=“Description of Unknown: miR-126-3p binding site sequence”
cgcauuauuacucacgguacga

SEQ ID NO: 122 - RNA, 21 nt, Unknown
  source: /note=“Description of Unknown: miR-126-5p sequence”
cauuauuacuuuugguacgcg

SEQ ID NO: 123 - RNA, 21 nt, Unknown
  source: /note=“Description of Unknown: miR-126-5p binding site sequence”
cgcguaccaaaaguaauaaug

SEQ ID NO: 124 - RNA, 9 nt, Artificial Sequence
  source: /note=“Description of Artificial Sequence: Synthetic oligonucleotide”
ugauaauag

SEQ ID NO: 125 - RNA, 9 nt, Artificial Sequence
  source: /note=“Description of Artificial Sequence: Synthetic oligonucleotide”
ugauaguaa

SEQ ID NO: 126 - RNA, 9 nt, Artificial Sequence
  source: /note=“Description of Artificial Sequence: Synthetic oligonucleotide”
uaaugauag

SEQ ID NO: 127 - RNA, 9 nt, Artificial Sequence
  source: /note=“Description of Artificial Sequence: Synthetic oligonucleotide”
ugauaauaa

SEQ ID NO: 128 - RNA, 9 nt, Artificial Sequence
  source: /note=“Description of Artificial Sequence: Synthetic oligonucleotide”
ugauaguag

SEQ ID NO: 129 - RNA, 9 nt, Artificial Sequence
  source: /note=“Description of Artificial Sequence: Synthetic oligonucleotide”
uaaugauga

SEQ ID NO: 130 - RNA, 9 nt, Artificial Sequence
  source: /note=“Description of Artificial Sequence: Synthetic oligonucleotide”
uaauaguag

SEQ ID NO: 131 - RNA, 9 nt, Artificial Sequence
  source: /note=“Description of Artificial Sequence: Synthetic oligonucleotide”
ugaugauga

SEQ ID NO: 132 - RNA, 9 nt, Artificial Sequence
  source: /note=“Description of Artificial Sequence: Synthetic oligonucleotide”
uaauaauaa

SEQ ID NO: 133 - RNA, 9 nt, Artificial Sequence
  source: /note=“Description of Artificial Sequence: Synthetic oligonucleotide”
uaguaguag

SEQ ID NO: 134 - RNA, 133 nt, Artificial Sequence
  source: /note=“Description of Artificial Sequence: Synthetic polynucleotide”
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 135 - RNA, 22 nt, Unknown
  source: /note=“Description of Unknown: miR 146-3p sequence”
ccucugaaauucaguucuucag

SEQ ID NO: 136 - RNA, 22 nt, Unknown
  source: /note=“Description of Unknown: miR 146-5p sequence”
ugagaacugaauuccauggguu

SEQ ID NO: 137 - RNA, 22 nt, Unknown
  source: /note=“Description of Unknown: miR 155-3p sequence”
cuccuacauauuagcauuaaca

SEQ ID NO: 138 - RNA, 23 nt, Unknown
  source: /note=“Description of Unknown: miR 155-5p sequence”
uuaaugcuaaucgugauaggggu

SEQ ID NO: 139 - RNA, 22 nt, Unknown
  source: /note=“Description of Unknown: miR 16-3p sequence”
ccaguauuaacugugcugcuga

SEQ ID NO: 140 - RNA, 22 nt, Unknown
  source: /note=“Description of Unknown: miR 16-5p sequence”
uagcagcacguaaauauuggcg

SEQ ID NO: 141 - RNA, 21 nt, Unknown
  source: /note=“Description of Unknown: miR 21-3p sequence”
caacaccagucgaugggcugu

SEQ ID NO: 142 - RNA, 22 nt, Unknown
  source: /note=“Description of Unknown: miR 21-5p sequence”
uagcuuaucagacugauguuga

SEQ ID NO: 143 - RNA, 22 nt, Unknown
  source: /note=“Description of Unknown: miR 223-3p sequence”
ugucaguuugucaaauacccca

SEQ ID NO: 144 - RNA, 22 nt, Unknown
  source: /note=“Description of Unknown: miR 223-5p sequence”
cguguauuugacaagcugaguu

SEQ ID NO: 145 - RNA, 22 nt, Unknown
  source: /note=“Description of Unknown: miR 24-3p sequence”
uggcucaguucagcaggaacag

SEQ ID NO: 146 - RNA, 22 nt, Unknown
  source: /note=“Description of Unknown: miR 24-5p sequence”
ugccuacugagcugauaucagu

SEQ ID NO: 147 - RNA, 21 nt, Unknown
  source: /note=“Description of Unknown: miR 27-3p sequence”
uucacaguggcuaaguuccgc

SEQ ID NO: 148 - RNA, 22 nt, Unknown
  source: /note=“Description of Unknown: miR 27-5p sequence”
agggcuuagcugcuugugagca

SEQ ID NO: 149 - RNA, 141 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 150 - RNA, 119 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaagucugagugggcggc

SEQ ID NO: 151 - RNA, 142 nt, Artificial Sequence; identical to SEQ ID NO: 109
  source: /note=“Description of Artificial Sequence: Synthetic polynucleotide”

SEQ ID NO: 152 - skipped

SEQ ID NO: 153 - RNA, 23 nt, Unknown
  source: /note=“Description of Unknown: miR 155-5p sequence”
uuaaugcuaauugugauaggggu

SEQ ID NO: 154 - RNA, 23 nt, Unknown
  source: /note=“Description of Unknown: miR 155-5p binding site sequence”
accccuaucacaauuagcauuaa

SEQ ID NO: 155 - RNA, 188 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 156 - RNA, 140 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccaguagugcuuucuacuuuaugguggucuuugaauaaagucugagugggcggc

SEQ ID NO: 157 - RNA, 182 nt, Artificial Sequence
  source: /note=“Description of Artificial Sequence: Synthetic polynucleotide”
ugauaauagaguagugcuuucuacuuuauggcuggagccucgguggccaugcuucuugccccuugggccaguagugcuuucuacuuuauguccccccagccccuccuccccuuccugcacccguacccccaguagugcuuucuacuuuaugguggucuuugaauaaagucugagugggcggc

SEQ ID NO: 158 - RNA, 184 nt, Artificial Sequence
  source: /note=“Description of Artificial Sequence: Synthetic polynucleotide”
ugauaauagaguagugcuuucuacuuuauggcuggagccucgguggccaugcuucuugccccuugggccuccauaaaguaggaaacacuacauccccccagccccuccuccccuuccugcacccguacccccaguagugcuuucuacuuuaugguggucuuugaauaaagucugagugggcggc

SEQ ID NO: 159 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccaccccuaucacaauuagcauuaaguggucuuugaauaaagucugagugggcggc

SEQ ID NO: 160 - RNA, 188 nt, Artificial Sequence
  source: /note=“Description of Artificial Sequence: Synthetic polynucleotide”
ugauaauagaccccuaucacaauuagcauuaagcuggagccucgguggccaugcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 161 - RNA, 188 nt, Artificial Sequence
  source: /note=“Description of Artificial Sequence: Synthetic polynucleotide”
ugauaauagaccccuaucacaauuagcauuaagcuggagccucgguggccaugcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 162 - RNA, 142 nt, Artificial Sequence; identical to SEQ ID NO: 104
  source: /note=“Description of Artificial Sequence: Synthetic polynucleotide”

SEQ ID NO: 163 - RNA, 142 nt, Artificial Sequence; identical to SEQ ID NO: 105
  source: /note=“Description of Artificial Sequence: Synthetic polynucleotide”

SEQ ID NO: 164 - RNA, 142 nt, Artificial Sequence
  source: /note=“Description of Artificial Sequence: Synthetic polynucleotide”
ugauaauaggcuggagccucgguggccaugcuucuugccccuugggccuccauaaaguaggaaacacuacauccccccagccccuccuccccuuccugcacccguacccccguggucuuugaauaaagucugagugggcggc

SEQ ID NO: 165 - RNA, 70 nt, Artificial Sequence
  source: /note=“Description of Artificial Sequence: Synthetic oligonucleotide”
gggaaauaagaguccauaaaguaggaaacacuacaagaaaagaagaguaagaagaaauauaagagccacc

SEQ ID NO: 166 - RNA, 70 nt, Artificial Sequence
  source: /note=“Description of Artificial Sequence: Synthetic oligonucleotide”
gggaaauaagagagaaaagaagaguaauccauaaaguaggaaacacuacagaagaaauauaagagccacc

SEQ ID NO: 167 - RNA, 70 nt, Artificial Sequence
  source: /note=“Description of Artificial Sequence: Synthetic oligonucleotide”
gggaaauaagagagaaaagaagaguaagaagaaauauaauccauaaaguaggaaacacuacagagccacc

SEQ ID NO: 168 - RNA, 23 nt, Unknown
  source: /note=“Description of Unknown: miR 155-5p binding site sequence”
accccuaucacaauuagcauuaa

SEQ ID NO: 169 - RNA, 181 nt, Artificial Sequence
  source: /note=“Description of Artificial Sequence: Synthetic polynucleotide”
ugauaauagaguagugcuuucuacuuuauggcuggagccucgguggccaugcuucuugccccuugggccaguagugcuuucuacuuuauguccccccagccccucuccccuuccugcacccguacccccaguagugcuuucuacuuuaugguggucuuugaauaaagucugagugggcggc

SEQ ID NO: 170 - RNA, 142 nt, Artificial Sequence; identical to SEQ ID NO: 106
  source: /note=“Description of Artificial Sequence: Synthetic polynucleotide”

SEQ ID NO: 171 - RNA, 142 nt, Artificial Sequence; identical to SEQ ID NO: 107
  source: /note=“Description of Artificial Sequence: Synthetic polynucleotide”

SEQ ID NO: 172 - RNA, 142 nt, Artificial Sequence; identical to SEQ ID NO: 108
  source: /note=“Description of Artificial Sequence: Synthetic polynucleotide”

SEQ ID NO: 173 - RNA, 142 nt, Artificial Sequence; identical to SEQ ID NO: 110
  source: /note=“Description of Artificial Sequence: Synthetic polynucleotide”

SEQ ID NO: 174 - RNA, 164 nt, Artificial Sequence; identical to SEQ ID NO: 112
  source: /note=“Description of Artificial Sequence: Synthetic polynucleotide”

SEQ ID NO: 175 - RNA, 119 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccguggucuuugaauaaagucugagugggcggc

SEQ ID NO: 176 - skipped

SEQ ID NO: 177 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 178 - RNA, 188 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 179 - RNA, 142 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccccccagccccuccuccccuuccugcacccguacccccguggucuuugaauaaagucugagugggcggc

SEQ ID NO: 180 - RNA, 142 nt, Artificial Sequence
  source: /note=“Description of Artificial Sequence: Synthetic polynucleotide”
ugauaauaggcuggagccucgguggcuccauaaaguaggaaacacuacacuagcuucuugccccuugggccuccccccagccccuccuccccuuccugcacccguacccccguggucuuugaauaaagucugagugggcggc

SEQ ID NO: 181 - RNA, 142 nt, Artificial Sequence
  source: /note=“Description of Artificial Sequence: Synthetic polynucleotide”
ugauaauaggcuggagccucgguggccuagcuucuugccccuugggccuccauaaaguaggaaacacuacauccccccagccccuccuccccuuccugcacccguacccccguggucuuugaauaaagucugagugggcggc

SEQ ID NO: 182 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccaccccuaucacaauuagcauuaaguggucuuugaauaaagucugagugggcggc

SEQ ID NO: 183 - RNA, 188 nt, Artificial Sequence
  source: /note=“Description of Artificial Sequence: Synthetic polynucleotide”
ugauaauagaccccuaucacaauuagcauuaagcuggagccucgguggccuagcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 184 - RNA, 188 nt, Artificial Sequence
  source: /note=“Description of Artificial Sequence: Synthetic polynucleotide”
ugauaauagaccccuaucacaauuagcauuaagcuggagccucgguggccuagcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 185 - RNA, 47 nt, Unknown
  source: /note=“Description of Unknown: 5′ UTR sequence”
gggaaauaagagagaaaagaagaguaagaagaaauauaagagccacc

SEQ ID NO: 186 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 187 - protein, 740 aa, Artificial Sequence
  source: /note=“Description of Artificial Sequence: Synthetic polypeptide”
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 188 - RNA, 2220 nt, Artificial Sequence
  source: /note=“Description of Artificial Sequence: Synthetic polynucleotide”
augaagugggugaccuucaucagccugcuguuccuguucagcagcgccuacagcagaggcguguucagaagagacgcccacaagagcgagguggcccacagauucaaggaccugggcgaggagaacuucaaggcccuggugcugaucgccuucgcccaguaccugcagcagugcccuuucgaggaccacgugaagcuggugaacgaggugaccgaguucgccaagaccugcguggccgacgagagcgccgagaacugcgacaagagccugcacacccuguucggcgacaagcugugcaccguggccacccugagagaaacuuacggcgagauggccgacugcugcgccaagcaggagccagagcggaacgagugcuuccugcaacacaaggacgacaacccuaaccugccuagacugguccggccugagguggacgugaugugcacggccuuccacgacaacgaggagacuuuccugaagaaguaccuguacgagaucgccagaagacacccuuacuucuacgccccugagcuccuguucuucgcgaagagauacaaggccgccuucaccgagugcugccaggccgccgacaaggcagcuugccugcugccuaagcuggacgagcugagagacgagggcaaggccuccucagcuaagcagagacugaagugcgccagccugcagaaguucggugagagagcauucaaggcuugggccgucgcaagacugucacagagauucccuaaggcagaauucgcggaggugagcaagcuagugaccgaccugaccaaggugcauacagagugcugccacggcgaccugcuggagugcgccgacgacagagccgaccuggccaaguacaucugcgagaaccaggacagcaucagcuccaagcugaaggagugcugugagaagccucugcuggagaagucacacugcauugccgaggucgagaacgacgagaugccugccgaucuuccuagccuugccgccgauuucguggagagcaaggacgugugcaagaacuacgccgaggcaaaggacguguuccugggcauguuccuuuacgaauacgcucgccggcauccagacuacagcguggugcugcugcugagauuggccaagacuuacgagacgacccucgagaaguguugcgcagcagcugauccucacgaguguuacgccaagguguucgacgaguucaagccgcuuguggaggagccucagaaccugaucaagcagaauugugagcuguucgagcagcugggugaguacaaguuccagaacgcccugcuggugcgcuacaccaagaaggugccucaagugucuaccccuacccugguugaaguuucccgcaaccugggcaaggugggcagcaagugcugcaagcauccugaagcaaagaggaugccuugcgccgaggacuaccugucagugguccuuaaccagcugugcgugcugcacgagaagaccccugugagcgacagagugacaaaguguuguaccgagagccuggucaacagaagaccuugcuucagcgcccuggaagucgacgagacauacgugccuaaggaguucaacgccgaaaccuucaccuuccacgccgacaucugcacacugagcgagaaggagagacagaucaagaagcagaccgcccuggucgaguuggugaagcacaagccuaaggccaccaaggagcaacucaaggccgugauggacgacuucgcggccuucguugagaagugcuguaaggcugacgacaaggagacgugcuucgcugaggaggguaagaagcuugucgccgccucucaggccgcuuugggacucggcggcggcagugcgccugccagaagcccuuccccaucuacccagccuugggagcacgugaacgccauccaggaggccagacgucugcugaaccugucacgggauaccgcagcugagaugaacgaaacuguugaggucaucagcgagauguucgaccuacaggaaccuaccugcuugcagaccagacuggagcuguacaagcagggauuaagaggcucccugacgaagcuuaagggcccucugaccaugauggccagccacuauaagcagcacugcccuccuaccccugaaacgucgugugcuacccagaucaucaccuucgagagcuucaaggagaaucugaaggacuuccugcucguuauuccguucgauuguugggagccugugcaggag

SEQ ID NO: 189 - protein, 584 aa, Homo sapiens
AHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

(19) United States
(12) Patent Application Publication
(10) Pub. No.: US 2024/0123034 A1
(43) Pub. Date: Apr. 18, 2024

(54) MRNAS ENCODING GRANULOCYTE-MACROPHAGE COLONY STIMULATING FACTOR FOR TREATING PARKINSON'S DISEASE

(71) Applicants: ModernaTX, Inc., Cambridge, MA (US); BOARD OF REGENTS OF THE UNIVERSITY OF NEBRASKA, Lincoln, NE (US)

(72) Inventors: Eric Yi-Chun Huang, Boston, MA (US); Jared Iacovelli, Waltham, MA (US); Seymour De Picciotto, Boston, MA (US); Sze-Wah Tse, Boston, MA (US); Laurie Kenney, Boston, MA (US); Katherine Olson, Council Bluffs, IA (US); Howard Gendelman, Omaha, NE (US); Lee R. Mosley, Omaha, NE (US)

(21) Appl. No.: 17/768,514

(22) PCT Filed: Oct. 15, 2020
(86) PCT No.: PCT/US20/55852

§ 371 Date: Apr. 13, 2022

Related U.S. Application Data
(60) Provisional application No. 62/915,317, filed on Oct. 15, 2019.
(60) Provisional application No. 63/013,139, filed on Apr. 21, 2020.

(51) Int. Cl. A61K 38/19; A61K 9/51; A61K 31/7115; A61P 25/16

(52) U.S. Cl. CPC A61K 38/193; A61K 9/5123; A61K 31/7115; A61P 25/16

(57) ABSTRACT

The disclosure features lipid nanoparticle (LNP) compositions comprising mRNA molecules encoding human granulocyte macrophage colony stimulating factor (GM-CSF) polypeptides and uses thereof in the treatment of Parkinson's Disease. The LNP compositions of the present disclosure comprise mRNA therapeutics encoding human GM-CSF for use in such treatment.

### CROSS-REFERENCE TO RELATED APPLICATIONS

This application claims the benefit of U.S. Provisional Application No. 62/915,317, filed Oct. 15, 2019 and U.S. Provisional Application No. 63/013,139, filed Apr. 21, 2020. The contents of the aforesaid applications are hereby incorporated by reference in their entirety.

### GOVERNMENT SUPPORT

This invention was made with government support under P01 DA028555, R01 NS036126, P30 MH062261, R01 AG043540, and 2R01 NS034239 awarded by the National Institutes of Health. The government has certain rights in the invention.

### SEQUENCE LISTING

This application contains a Sequence Listing which has been submitted electronically in ASCII format and is hereby incorporated by reference in its entirety. Said ASCII copy, created on Oct. 15, 2020 is named M2180-7002WO_SL.txt and is 61,278 bytes in size.

### BACKGROUND OF THE DISCLOSURE

Regulatory T cells (also known as T regulatory cells or T regs) are of potential therapeutic value for prevention and/or treatment of stroke, amyotrophic lateral sclerosis (ALS), Alzheimer's and Parkinson's diseases. In Parkinson's Disease, disease onset and progression is often linked to diminished numbers of Tregs and their anti-proliferation and anti-inflammatory activities. However, the mechanism by which this occurs remains under investigation. Therefore, there is an unmet need to develop therapies that can stimulate regulatory T cells in Parkinson's disease.

### SUMMARY OF THE DISCLOSURE

The present disclosure provides, inter alia, methods of using a lipid nanoparticle (LNP) composition comprising a polynucleotide encoding a human granulocyte macrophage colony stimulating factor (GM-CSF) polypeptide for the treatment of Parkinson's disease. The LNP compositions of the present disclosure comprise mRNA therapeutics encoding a human GM-CSF polypeptide for use in the methods described herein. In an aspect, the LNP compositions of the present disclosure can be used in the treatment of Parkinson's disease in a subject. Additional aspects of the disclosure are described in further detail below.

Accordingly, in one aspect, the disclosure provides a lipid nanoparticle (LNP) composition comprising a polynucleotide encoding a human GM-CSF polypeptide for use, in the treatment of Parkinson's disease in a subject. In an embodiment, the GM-CSF polypeptide comprises the amino acid sequence of SEQ ID NO: 1. In an embodiment, the GM-CSF polypeptide comprises the amino acid sequence of SEQ ID NO: 8. In an embodiment, the GM-CSF polypeptide comprises the amino acid sequence of SEQ ID NO: 8 without the leader sequence. In an embodiment, the GM-CSF polypeptide comprises the amino acid sequence of SEQ ID NO: 187. In an embodiment, the GM-CSF polypeptide comprises the amino acid sequence of SEQ ID NO: 187 without the leader sequence.

In another aspect, provided herein is a method of treating Parkinson's disease in a subject, comprising administering to the subject an effective amount of a lipid nanoparticle (LNP) composition comprising a polynucleotide encoding a human GM-CSF polypeptide. In an embodiment, the GM-CSF polypeptide comprises the amino acid sequence of SEQ ID NO: 1. In an embodiment, the GM-CSF polypeptide comprises the amino acid sequence of SEQ ID NO: 8.

In an embodiment, the GM-CSF polypeptide comprises the amino acid sequence of SEQ ID NO: 8 without the leader sequence. In an embodiment, the GM-CSF polypeptide comprises the amino acid sequence of SEQ ID NO: 187. In an embodiment, the GM-CSF polypeptide comprises the amino acid sequence of SEQ ID NO: 187 without the leader sequence.

In an embodiment of a method or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 2. In an embodiment, the polynucleotide encoding the human GM-CSF polypeptide comprises the nucleotide sequence of SEQ ID NO: 2.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 3. In an embodiment, the polynucleotide encoding the human GM-CSF polypeptide comprises the nucleotide sequence of SEQ ID NO: 3.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 3. In an embodiment, the polynucleotide encoding the human GM-CSF polypeptide comprises the nucleotide sequence of SEQ ID NO: 188.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 4. In an embodiment, the polynucleotide encoding the human GM-CSF polypeptide comprises the nucleotide sequence of SEQ ID NO: 4.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 5. In an embodiment, the polynucleotide encoding the human GM-CSF polypeptide comprises the nucleotide sequence of SEQ ID NO: 5.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 6. In an embodiment, the polynucleotide encoding the human GM-CSF polypeptide comprises the nucleotide sequence of SEQ ID NO: 6.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 7. In an embodiment, the polynucleotide encoding the human GM-CSF polypeptide comprises the nucleotide sequence of SEQ ID NO: 7.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to the sequence of SEQ ID NO: 12. In an embodiment, the polynucleotide encoding the human GM-CSF polypeptide comprises the nucleotide sequence of SEQ ID NO: 12.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 216.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 221.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 219.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 224.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 201.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 204.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 206.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 209.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 211.

In an embodiment of any of the methods or composition for use disclosed herein, the polynucleotide encoding the human GM-CSF polypeptide comprises a nucleotide sequence having at least 85%, 90%, 95%, 96%, 97%, 98%, 99% or 100% identity to SEQ ID NO: 214.

In an embodiment of any of the methods or composition for use disclosed herein, administration of LNP increases the level and/or activity of T regulatory cells in a sample (e.g., a sample from a subject), e.g., as determined by an assay in any one of Examples 2-8.

In an embodiment of any of the methods or composition for use disclosed herein, the T regulatory cells comprise FoxP3+ expressing and/or CD25+ expressing T regulatory cells. In an embodiment, the T regulatory cells comprise FoxP3+ expressing T regulatory cells. In an embodiment, the T regulatory cells comprise CD25+ expressing T regulatory cells. In an embodiment, the T regulatory cells comprise FoxP3+ expressing and CD25+ expressing T regulatory cells.

In an embodiment, the T regulatory cells are CD4+ and/or CD8+ T regulatory cells. In an embodiment, the T regulatory cells are CD4+ T regulatory cells. In an embodiment, the T regulatory cells are CD8+ T regulatory cells. In an embodiment, the T regulatory cells are CD4+ and CD8+ T regulatory cells.

In an embodiment of any of the methods or composition for use disclosed herein, the increase in level and/or activity of T regulatory cells is compared to the level and/or activity of T regulatory cells in an otherwise similar sample which is: not contacted with the LNP; or contacted with recombinant GM-CSF. In an embodiment, the increase in level and/or activity of T regulatory cells occurs in vivo.

In an embodiment of any of the methods or composition for use disclosed herein, the increase in level and/or activity of T regulatory cells comprises one, two, or all, or a combination of the following parameters:
(a) increased level of (e.g., number or proportion of) T regulatory cells (e.g., CD4+ FoxP3+CD25+ T regulatory cells);
(b) increased activity or expression level of one or more genes listed in FIG. 6A, or one or more pathways listed in FIG. 6B or FIG. 6C; or
(c) decreased activity or expression level of one or more genes listed in FIG. 6A, or one or more pathways listed in FIG. 6B or FIG. 6C.

In an embodiment, the increase in level and/or activity of T regulatory cells comprises increased activity or expression level of one or more genes listed in FIG. 6A, or one or more pathways listed in FIG. 6B or FIG. 6C. In an embodiment, the increase in level and/or activity of T regulatory cells comprises increased activity or expression level of one or more genes listed in FIG. 6A. In an embodiment, the increase in activity and/or expression level is about 2-5 fold, about 2-4.5 fold, about 2-4 fold, about 2-3.5 fold, or about 2-3 fold. In an embodiment, the increase in activity and/or expression level is about 2 fold. In an embodiment, the increase in activity and/or expression level is about 3 fold. In an embodiment, the increase in activity and/or expression level is about 4 fold. In an embodiment, the increase in activity and/or expression level is about 5 fold. In an embodiment, the increase in activity and/or expression level is more than 5-fold.

In an embodiment of any of the methods or composition for use disclosed herein, administration of the LNP comprising a polynucleotide encoding GM-CSF increases bioavailability of GM-CSF (e.g., in a sample from the subject). In an embodiment, the increase in bioavailability is compared to administration of recombinant GM-CSF, e.g., sargramostim. In an embodiment, the increase in bioavailability is at least 1.5 to 10 fold, at least 1.5 to 9 fold, at least 1.5 to 8 fold, at least 1.5 to 7 fold, at least 1.5 to 6 fold, at least 1.5 to 5 fold, at least 1.5 to 4 fold, at least 1.5 to 3 fold or at least 1.5 to 2 fold.

In an embodiment of any of the methods or composition for use disclosed herein, administration of the LNP comprising a polynucleotide encoding GM-CSF increases the expression level, e.g., stability or half-life, of GM-CSF (e.g., in a plasma sample from the subject), as compared to: a subject who has not been administered the LNP comprising a polynucleotide encoding GM-CSF; or a subject who has been administered recombinant GM-CSF, e.g., Sargramostim. the increase in expression level of GM-CSF is about 10 to 50 fold, e.g., as measured by an assay in Example 2. In an embodiment, the increase in expression level of GM-CSF is about 10-45 fold, about 10-40 fold, about 10-35 fold, about 10-30 fold, about 10-25 fold, about 10-20 fold or about 10-15 fold.

In an embodiment of any of the methods or composition for use disclosed herein, the level of GM-CSF in tissues is not increased as compared to a reference, e.g., an appropriate control.

In an embodiment of any of the methods or composition for use disclosed herein, the LNP comprising an mRNA encoding GM-CSF can be administered at a lower dose (e.g., lower effective dose) as compared to the dose of GM-CSF administered in a different form. In an embodiment, the lower dose of the LNP is compared to a dose of recombinant GM-CSF, e.g., Sargramostim. In an embodiment, the dose of GM-CSF in the LNP is at least 1.5 fold, 2 fold, 3 fold, 4 fold, 5 fold, 6 fold, 7 fold, 8 fold, 9 fold or 10 fold lower. In an embodiment, the dose of GM-CSF in the LNP is about 1.5 to 10 fold lower, about 1.5 to 9 fold lower, about 1.5 to 8 fold lower, about 1.5 to 7 fold lower, about 1.5 to 6 fold lower, about 1.5 to 5 fold lower, about 1.5 to 4 fold lower, about 1.5 to 3 fold lower, or about 1.5 fold to 2 fold lower, e.g., compared to a dose of recombinant GM-CSF, e.g., Sargramostim.

In an embodiment of any of the methods or composition for use disclosed herein, administration of the LNP prevents a reduction in the level of neurons (e.g., number or proportion of neurons), e.g., as compared to the level of neurons in a subject who has not been administered the LNP comprising a polynucleotide encoding GM-CSF; or a subject who has been administered recombinant GM-CSF, e.g., Sargramostim. In an embodiment, the level of, e.g., number of, neurons is at least 20-50% higher in a sample from the subject administered the LNP, e.g., as measured by an assay in Example 4 or 5.

In an embodiment, nigrostriatal neurodegeneration and/or microglial activation is reduced upon administration of an LNP comprising an mRNA encoding GM-CSF, e.g., as compared to a reference, e.g., an appropriate control. In an embodiment, the reduction in nigrostriatal neurodegeneration and/or microglial activation is about at least 1.5 fold, 2 fold, 3 fold, 4 fold, 5 fold, 6 fold, 7 fold, 8 fold, 9 fold or 10 fold lesser as compared to nigrostriatal neurodegeneration and/or microglial activation in a reference, e.g., without administration of an LNP comprising an mRNA encoding GM-CSF.

In an embodiment of any of the methods or composition for use disclosed herein, the polynucleotide encoding the human GM-CSF polypeptide comprises at least one chemical modification. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine.

In an embodiment of any of the methods or composition for use disclosed herein, the polynucleotide encoding the human GM-CSF polypeptide comprises an mRNA comprising fully modified N1-methylpseudouridine. In an embodiment of any of the methods or composition for use disclosed herein, the polynucleotide encoding the human GM-CSF polypeptide comprises an otherwise identical mRNA that does not comprise one or more or fully modified N1-methylpseudouridine.

In an embodiment of any of the methods or composition for use disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid, e.g., a PEG-modified lipid. In an embodiment, the ionizable lipid comprises Compound 18. In an embodiment, the phospholipid comprises Compound H-409. In an embodiment, the structural lipid comprises cholesterol. In an embodiment, the PEG-lipid comprises PEG-DMG or Compound P-428.

Nucleic acids of the present disclosure (encoding GM-CSF mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In an embodiment, the LNP comprises a molar ratio of about 20-60% Compound 18: 5-25% phospholipid: 25-55% cholesterol; and 0.5-15% PEG-modified lipid. In an embodiment, the LNP comprises a molar ratio of about 50% Compound 18: about 10% phospholipid: about 38.5% cholesterol; and about 1.5% PEG-modified lipid.

In an embodiment, the LNP comprises a molar ratio of about 50% Compound 18: about 10% Compound H-409: about 38.5% cholesterol; and about 1.5% PEG-DMG.

In an embodiment, the LNP comprises a molar ratio of about 50% Compound 18: about 10% Compound H-409: about 38.5% cholesterol; and about 1.5% Compound P-428.

In an embodiment of any of the methods or composition for use disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid, e.g., a PEG-modified lipid. In an embodiment, the ionizable lipid comprises Compound 25. In an embodiment, the phospholipid comprises Compound H-409. In an embodiment, the structural lipid comprises cholesterol. In an embodiment, the PEG-lipid comprises PEG-DMG or Compound P-428.

In an embodiment, the LNP comprises a molar ratio of about 20-60% Compound 25: 5-25% phospholipid: 25-55% cholesterol; and 0.5-15% PEG-modified lipid. In an embodiment, the LNP comprises a molar ratio of about 50% Compound 25: about 10% phospholipid: about 38.5% cholesterol; and about 1.5% PEG-modified lipid.

In an embodiment, the LNP comprises a molar ratio of about 50% Compound 25: about 10% Compound H-409: about 38.5% cholesterol; and about 1.5% PEG-DMG.

In an embodiment, the LNP comprises a molar ratio of about 50% Compound 25: about 10% Compound H-409: about 38.5% cholesterol; and about 1.5% Compound P-428.

In an embodiment of any of the methods or composition for use disclosed herein, the subject administered the LNP is a mammal, e.g., a mouse, rat or a human. In an embodiment, the subject is a human.

In an embodiment of any of the methods or composition for use disclosed herein, the composition is administered intramuscularly or subcutaneously. In an embodiment, the composition is administered intramuscularly. In an embodiment, the composition is administered subcutaneously.

In an embodiment of any of the methods or composition for use disclosed herein, the LNP is administered daily for about 2-35 days, e.g., about 2, 3, 4, 5, 6, 7, 8, 9, 10, 14, 21, 28, 30 or 35 days. In an embodiment, the LNP is administered daily for about 2-35 days, about 2-34 days. about 2-33 days, about 2-32 days, about 2-31 days, about 2-30 days, about 2-29 days, about 2-28 days, about 2-27 days, about 2-26 days, about 2-25 days, about 2-24 days, about 2-23 days, about 2-22 days, about 2-21 days, about 2-20 days, about 2-19 days, about 2-18 days, about 2-17 days, about 2-16 days, about 2-15 days, about 2-14 days, about 2-13 days, about 2-12 days, about 2-11 days, about 2-10 days, about 2-9 days, about 2-8 days, about 2-7 days, about 2-6 days, about 2-5 days, about 3-35 days, about 5-35 days, about 10-35 days, about 14-35 days, about 21-35 days, about 28-35 days, about 30-35 days, or about 21-30 days. In an embodiment, the LNP is administered daily for about 4 days. In an embodiment, the LNP is administered daily for about 28 days. In another embodiment, the LNP is administered less frequently, e.g., weekly, every other week, monthly, or less frequently.

In an embodiment of any of the methods or composition for use disclosed herein, the LNP is administered as a monotherapy.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### Other Embodiments of the Disclosure

E1. A lipid nanoparticle (LNP) composition comprising a polynucleotide encoding a human GM-CSF polypeptide for use, in the treatment of Parkinson's disease in a subject, wherein the GM-CSF polypeptide comprises the amino acid sequence of SEQ ID NO: 1, SEQ ID NO: 8, SEQ ID NO: 187, SEQ ID NO: 215, or SEQ ID NO: 220.
E2. A method of treating Parkinson's disease in a subject, comprising administering to the subject an effective amount of a lipid nanoparticle (LNP) composition comprising a polynucleotide encoding a human GM-CSF polypeptide, wherein the GM-CSF polypeptide comprises the amino acid sequence of SEQ ID NO: 1, SEQ ID NO: 8, SEQ ID NO: 187, SEQ ID NO: 215, or SEQ ID NO: 220.
E3. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 2.
E4. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 3.
E5. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 4.
E6. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 5.
E7. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 6.
E8. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 7.
E9. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 188.
E10. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 216.
E11. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 219.
E12. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 221.
E13. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 224.
E14. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 201 or 204.
E15. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 206 or 209.
E16. The LNP composition for use of embodiment 1, or the method of embodiment 2, wherein the polynucleotide encoding the human GM-CSF polypeptide comprises a nucleotide sequence having at least 85%, 90%, 95%, 96%, 97%, 98%, 99% or 100% identity to the sequence of SEQ ID NO: 211 or 214.
E17. The LNP composition for use, or the method of any one of embodiments 1-16, wherein administration of LNP increases the level and/or activity of T regulatory cells in a sample (e.g., a sample from a subject), e.g., as determined by an assay in any one of Examples 2-8.
E18. The LNP composition for use, or the method of embodiment 17, wherein the T regulatory cells comprise FoxP3+ expressing and/or CD25+ expressing T regulatory cells.
E19. The LNP composition for use, or the method of embodiment 17 or 18, wherein the T regulatory cells are CD4+ and/or CD8+ T regulatory cells.
E20. The LNP composition for use, or the method of any one of embodiments 17 to 19, wherein the increase in level and/or activity of T regulatory cells is compared to the level and/or activity of T regulatory cells in an otherwise similar sample which is: not contacted with the LNP; or contacted with recombinant GM-CSF.
E21. The LNP composition for use, or the method of any one of embodiments 17 to 20, wherein the increase in level and/or activity of T regulatory cells occurs in vivo.
E22. The LNP composition for use, or the method of any one of embodiments 17 to 21, wherein the increase in level and/or activity of T regulatory cells comprises one, two, or all, or a combination of the following parameters:
(a) increased level of (e.g., number or proportion of) T regulatory cells (e.g., CD4+ FoxP3+ CD25+ T regulatory cells);
(b) increased activity or expression level of one or more genes listed in FIG. 6A, or one or more pathways listed in FIG. 6B or FIG. 6C; or
(c) decreased activity or expression level of one or more genes listed in FIG. 6A, or one or more pathways listed in FIG. 6B or FIG. 6C.
   E23. The LNP composition for use, or the method of any one of embodiments 17 to 22, wherein the increase in level and/or activity of T regulatory cells is about 1.5-5 fold, e.g., as measured by an assay in any one of Examples 2-8.
   E24. The LNP composition for use, or the method of any one of embodiments 17-22, wherein the increase in activity and/or expression level of one or more genes listed in FIG. 6A is about 2-5 fold or more than 5-fold.
   E25. The LNP composition for use, or the method of any one of embodiments 17 to 22, wherein the decrease in activity and/or expression level of one or more genes listed in FIG. 6A is about 2-fold.
   E26. The LNP composition for use, or the method of any one of embodiments 1 to 25, wherein administration of the LNP comprising a polynucleotide encoding GM-CSF increases bioavailability of GM-CSF (e.g., in a sample from the subject), e.g., as compared to administration of recombinant GM-CSF, e.g., sargramostim.
   E27. The LNP composition for use, or the method of embodiment 26, wherein the increase in bioavailability is at least 1.5-10 fold.
   E28. The LNP composition for use, or the method of embodiment 26 or 27, wherein administration of the LNP comprising a polynucleotide encoding GM-CSF increases the expression level, e.g., stability or half-life, of GM-CSF (e.g., in a plasma sample from the subject), as compared to: a subject who has not been administered the LNP comprising a polynucleotide encoding GM-CSF; or a subject who has been administered recombinant GM-CSF, e.g., sargramostim.
   E29. The LNP composition for use, or the method of embodiment 28, wherein the increase in expression level of GM-CSF is about 10-50 fold, e.g., as measured by an assay in Example 2.
   E30. The LNP composition for use, or the method of any one of embodiments 26 to 29, wherein the LNP can be administered at a lower dose (e.g., lower effective dose), as compared to administration of recombinant GM-CSF, e.g., Sargramostim.
   E31. The LNP composition for use, or the method of embodiment 30, wherein the dose of GM-CSF in the LNP is at least 1.5 fold, 2 fold, 3 fold, 4 fold, 5 fold, 6 fold, 7 fold, 8 fold, 9 fold or 10 fold lower.
   E32. The LNP composition for use, or the method of any one of embodiments 1 to 31, wherein administration of the LNP prevents a reduction in the level of neurons (e.g., number or proportion of neurons), e.g., as compared to the level of neurons in a subject who has not been administered the LNP comprising a polynucleotide encoding GM-CSF; or a subject who has been administered recombinant GM-CSF, e.g., Sargramostim.
   E33. The LNP composition for use, or the method of embodiment 32, wherein the level of, e.g., number of, neurons is at least 20-50% higher in a sample from the subject administered the LNP, e.g., as measured by an assay in Example 4 or 5.
   E34. The LNP composition for use, or the method of any one of embodiments 1-33, wherein the polynucleotide encoding the human GM-CSF polypeptide comprises at least one chemical modification.
   E35. The LNP composition for use, or the method of embodiment 34, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   E36. The LNP composition for use, or the method of embodiment 35, wherein the chemical modification is selected from the group consisting of pseudouridine, N1-methylpseudouridine, 5-methylcytosine, 5-methoxyuridine, and a combination thereof.
   E37. The LNP composition for use, or the method of embodiment 35 or 36 wherein the chemical modification is N1-methylpseudouridine.
   E38. The LNP composition for use, or the method of any one of embodiments 1 to 37, wherein the polynucleotide comprises an mRNA comprising fully modified N1-methylpseudouridine.
   E39. The LNP composition for use, or the method of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid, e.g., a PEG-modified lipid.
   E40. The LNP composition for use, or the method of embodiment 39, wherein the ionizable lipid comprises Compound 18 or Compound 25.
   E41. The LNP composition for use, or the method of embodiment 39 or 40, wherein the phospholipid is Compound H-409.
   E42. The LNP composition for use, or the method of any one of embodiments 39 to 41, wherein the structural lipid is cholesterol.
   E43. The LNP composition for use, or the method of any one of embodiments 39 to 42, wherein the PEG-lipid is PEG-DMG or Compound P-428.
   E44. The LNP composition for use, or the method of any one of embodiments 39 to 43, wherein the LNP comprises a molar ratio of about 20-60% Compound 18 or Compound 25: 5-25% phospholipid: 25-55% cholesterol; and 0.5-15% PEG-modified lipid.
   E45. The LNP composition for use, or the method of embodiment 44, wherein the lipid nanoparticle comprises a molar ratio of about 50% Compound 18: about 10% phospholipid: about 38.5% cholesterol; and about 1.5% PEG-modified lipid.
   E46. The LNP composition for use, or the method of embodiment 44, wherein the lipid nanoparticle comprises a molar ratio of about 50% Compound 18: about 10% phospholipid: about 38.5% cholesterol; and about 1.5% PEG-DMG.
   E47. The LNP composition for use, or the method of embodiment 44, wherein the lipid nanoparticle comprises a molar ratio of about 50% Compound 18: about 10% phospholipid: about 38.5% cholesterol; and about 1.5% Compound P-428.
   E48. The LNP composition for use, or the method of embodiment 44, wherein the lipid nanoparticle comprises a molar ratio of about 50% Compound 25: about 10% phospholipid: about 38.5% cholesterol; and about 1.5% PEG-modified lipid.
   E49. The LNP composition for use, or the method of embodiment 44, wherein the lipid nanoparticle comprises a molar ratio of about 50% Compound 25: about 10% phospholipid: about 38.5% cholesterol; and about 1.5% PEG-DMG.
   E50. The LNP composition for use, or the method of embodiment 44, wherein the lipid nanoparticle comprises a molar ratio of about 50% Compound 25: about 10% phospholipid: about 38.5% cholesterol; and about 1.5% Compound P-428.
   E51. The LNP composition for use, or the method of any one of embodiments 1 to 50, wherein the subject is a mammal, e.g., a mouse, rat or a human.
   E52. The LNP composition for use, or the method of any one of embodiments 1 to 51, wherein the composition is administered intramuscularly or subcutaneously.
   E53. The LNP composition for use, or the method of any one of embodiments 1 to 52, wherein the LNP is administered daily for about 2-35 days, e.g., about 2, 3, 4, 5, 6, 7, 8, 9, 10, 14, 21, 28, 30 or 35 days.
   E54. The LNP composition for use, or the method of embodiment 53, wherein the LNP is administered daily for about 4 days.
   E55. The LNP composition for use, or the method of embodiment 53, wherein the LNP is administered daily for about 28 days.
   E56. The LNP composition for use, or the method of any one of embodiments 1 to 55, wherein the LNP is administered as a monotherapy.
   E57. The LNP composition for use, or the method of any one of embodiments 1 to 56, wherein the level of GM-CSF in tissues is not increased as compared to a reference, e.g., an appropriate control.
   E58. The LNP composition for use, or the method of any one of embodiments 1 to 57, nigrostriatal neurodegeneration and/or microglial activation is reduced upon administration of an LNP comprising an mRNA encoding GM-CSF, as compared to a reference, e.g., an appropriate control.
   E59. The LNP composition for use, or the method of embodiment 58, wherein the reduction in nigrostriatal neurodegeneration and/or microglial activation is about at least 1.5 fold, 2 fold, 3 fold, 4 fold, 5 fold, 6 fold, 7 fold, 8 fold, 9 fold or 10 fold lesser as compared to nigrostriatal neurodegeneration and/or microglial activation in a reference, e.g., without administration of an LNP comprising an mRNA encoding GM-CSF.

### BRIEF DESCRIPTION OF THE DRAWINGS

FIGS. 1A-1J demonstrate the effects of intramuscular treatment with LNP formulated Gm-csf mRNA. FIG. 1A provides a graph depicting the quantification of plasma GM-CSF protein levels in peripheral blood before (pre) and 6 hours after (post) treatment with multiple ascending doses of LNP formulated Gm-csf mRNA scaffold or control NTFIX. FIG. 1B provides representative images depicting splenomegaly in spleens from mice treated with multiple ascending doses of LNP formulated Gm-csf mRNA scaffold. FIG. 1C provides a graph depicting quantification of organ weight four days after initial treatment, and linear regression analysis of organ weight (R2=0.4674, P=0.0009). FIGS. 1D-1G provide graphs depicting absolute counts of white blood cells (WBC) (FIG. 1D), monocytes (FIG. 1E), neutrophils (FIG. 1F), and lymphocytes (FIG. 1G) within whole blood following treatment. FIGS. 1H-1J provide graphs depicting changes in blood chemistry profiles for alkaline phosphatase (FIG. 1H), albumin (FIG. 1I) and amylase (FIG. 1J) following treatment. Differences in mean±SEM (n=4-5 per group) were determined where p<0.05 compared with *pre, and ^{a}0 mg/kg.

FIGS. 2A-2G demonstrate increase in CD4+CD25+FOXP3+ Treg numbers in C57/BL6 mice with increasing doses of LNP formulated Gm-csf mRNA. FIG. 2A provides a graph depicting CD4+ T cell frequency in peripheral blood following treatment. FIG. 2B provides a graph depicting Treg frequency in peripheral blood follows Gm-csf mRNA treatment (n=4-5, R2=0.37, P=0.006). FIGS. 2C-2F provides graphs depicting quantification of CD3+ (FIG. 2C), CD8+ (FIG. 2D), CD4+ (FIG. 2E), and CD4+CD25+FOXP3+ (FIG. 2F) cells in peripheral blood following treatment with ascending doses of Gm-csf mRNA compared to treatment with native recombinant GM-CSF protein. FIG. 2G provides a graph depicting assessment of Treg-mediated inhibition (±SEM) of CFSE-stained Tresps (CD4+CD25−) stimulated with anti-CD3/CD28 beads. Tregs were isolated from untreated (0 mg/kg), Gm-csf mRNA-treated (0.001 mg/kg-0.1 mg/kg), or recombinant GM-CSF protein-treated mice after 4 days of treatment. Linear regression analysis indicates p<0.04, R2≥0.91 for 0 mg/kg, 0.001 mg/kg, 0.01 mg/kg, and GM-CSF lines and p=0.08, R2=0.84 for 0.1 mg/kg. Differences in mean±SEM (n=4-5 per group) were determined where p<0.05 compared with ^{a}0 mg/kg.

FIGS. 3A-3G show increases in myeloid population in a dose-dependent manner in C57/BL6 mice upon LNP formulated GM-CSF mRNA treatment. FIGS. 3A and 3B show the frequency of CD11c+ and representative flow cytometry plots gated on total CD45+CD3− population in treated animals. FIGS. 3C and 3D show the frequency and representative flow cytometry plots of CD11b+ or CD8a subpopulation within total CD11c+ population. FIGS. 3E and 3F show the frequency and representative flow cytometry plots of CD11 b+ population within total CD45+CD3− population. FIG. 3G shows the expression (represented by MFI) of CD86, CD40 and class II I-A/I-E on gated total CD11c+ cells. Results shown are mean+/−SEM from at least three independent experiments (FIGS. 3A-3F) and two (FIG. 3G) independent experiments.

FIGS. 4A-4E show attenuation of MPTP-induced nigrostriatal neurodegeneration and microglial activation in LNP formulated GM-CSF mRNA treated animals in an alpha-syn model. FIG. 4A provides photomicrographs of dopaminergic (TH+/Nissl+) neurons within the substantia nigra (SN) and TH+ cell termini within the striatum (STR) of mice (SN TH+=scale bar, 500 μm, STR=scale bar, 1000 μm). FIG. 4B provides a graph depicting stereological quantification of total numbers of surviving TH+/Nissl+ and non-dopaminergic (TH−/Nissl+) neurons within the SN following MPTP intoxication. Differences in means (±SEM, n=8) were determined where p<0.05 compared with: groups treated with PBS (indicated by the letter “a” in drawings); groups treated with MPTP (indicated by the letter “b” in drawings); groups treated with 0.01 mg/kg LNP formulated GM-CSF mRNA (indicated by the letter “c” in drawings), and groups treated with 0.1 mg/kg LNP formulated GM-CSF mRNA (indicated by the letter “d” in the drawings). Mean percent remaining total neuron number is indicated on each treatment bar.

FIG. 4C provides a graph depicting relative fold change of TH density in striatal dopaminergic termini normalized to PBS controls. Differences in means (±SEM, n=8) were determined where p<0.05 compared with PBS (indicated by the letter “a” in drawings). FIG. 4D provides photomicrographs of Mac-1+ microglia in the SN. For all images, mice were treated with PBS, MPTP, 0.001 mg/kg Gm-csf mRNA, 0.01 mg/kg Gm-csf mRNA, 0.1 mg/kg Gm-csf mRNA, or 0.1 mg/kg recombinant GM-CSF protein (scale bar, 500 μm; inset image=200×). FIG. 4E provides a graph depicting the quantification of reactive microglia taken from midbrains two days post MPTP-intoxication. Differences in means (±SEM, n=6) were determined where p<0.05 compared with PBS (indicated by the letter “a” in the drawings) and MPTP (indicated by the letter “b” in the drawings).

FIGS. 5A-5C show that adoptive transfer of mRNA-induced Treg is protective against MPTP-induced lesions. FIG. 5A provides representative images of TH+/Nissl+ dopaminergic neurons within the substantia nigra (SN, top row), along with the projections into the striatum (STR, bottom row) of mice treated with PBS or MPTP followed by adoptive transfer of Treg isolated from mice treated with either 0.01 mg/kg Gm-csf mRNA or 0.1 mg/kg Gm-csf mRNA (SN TH+=scale bar, 500 μm, STR=scale bar, 1000 μm). FIG. 5B provides a graph depicting quantification of total numbers of surviving dopaminergic (TH+/Nissl+) and non-dopaminergic (TH−/Nissl+) neurons within the SN following MPTP intoxication and adoptive transfer of 1×10⁶ Treg. FIG. 5C provides a graph depicting densitometry analysis of TH+ cell termini within the STR with MPTP intoxication followed by adoptive transfer of Treg. Differences in means (±SEM, n=6-7) were determined where p<0.05 compared with PBS (indicated by the letter “a” in drawings).

FIGS. 6A-6C show gene expression patterns in CD4+ T cell populations following treatment with LNP formulated Gm-csfmRNA. FIG. 6A provides a table of genes upregulated or downregulated ≥2. Genes are grouped according to the extent of upregulation or downregulation, and denoted with various patterns as described in the legend of FIG. 6A. FIG. 6B provides a pathway analysis schematic of dysregulated genes within the Hematological System Development and Function Network. FIG. 6C provides a pathway analysis schematic of dysregulated genes within the Cellular and Tissue Development Network. Nodes patterned with cross-hatching indicate downregulated genes. Nodes patterned with dots and dashes indicate upregulated genes. Nodes lacking patterning indicate genes identified by ingenuity pathway analysis (IPA) that were not measured but are involved in each corresponding signaling pathway. Grey arrows indicate direct relationships between two connecting genes.

FIGS. 7A-7I show the results of LNP formulated Gm-csf mRNA treatment in α-syn overexpressed Sprague-Dawley rats. FIG. 7A provides a graph depicting quantification of spleen weight normalized to body weight following treatment with PBS, 0.01 mg/kg, or 0.1 mg/kg rat Gm-csf mRNA, or 0.1 mg/kg recombinant rat GM-CSF protein. Differences in means (±SEM, n=3) were determined where p<0.05 compared with 0 mg/kg (indicated by the letter “a” in drawings). FIGS. 7B-7D provide graphs depicting flow cytometric analysis of T cell phenotype frequencies including CD3+ (FIG. 7B), CD4+ (FIG. 7C), and CD4+CD25+FOXP3+ (FIG. 7D) subsets in peripheral blood following treatment. Differences in means (±SEM, n=3) were determined where p<0.05 compared with 0 mg/kg (indicated by the letter “a” in drawings) and 0.1 mg/kg GM-CSF (indicated by the letter “b” in drawings). FIG. 7E provides a graph depicting quantification of Treg (CD4+CD25+)-mediated cell suppression (±SEM) at various Tresp:Treg ratios ex vivo. Treg-mediated suppression is expressed as percent inhibition. Linear regression analysis indicates p<0.01, R2≥0.87 for all treatments. FIGS. 7F-7I provide graphs depicting flow cytometric analysis of T cell phenotype frequencies before (pre) (black bars) and after (post) (gray bars) treatment with either Sham, AAV2/1-GFP (AAV-GFP) vector, AAV2/1-α-syn (AAV-α-syn) vector, AAV-α-syn+0.01 mg/kg Gm-csf mRNA, or AAV-α-syn+0.1 mg/kg Gm-csf mRNA. Peripheral whole blood was analyzed for the frequency of CD3+ (FIG. 7F), CD4+ (FIG. 7G), CD8+ (FIG. 7H), or CD4+CD25+FOXP3+ (FIG. 7I) cells within the lymphocyte population. Differences in means (±SEM, n=7) were determined where P<0.05 compared with *pre, Sham-post (indicated by the letter “a” in drawings), AAV-GFP-post (indicated by the letter “b” in drawings), AAV-α-syn-post (indicated by the letter “c” in drawings), or AAV-α-syn+0.01 mg/kg Gm-csf mRNA-post (indicated by the letter “d” in drawings).

FIGS. 8A-8E depict neuroprotective and anti-inflammatory effects of extended treatment with LNP formulated Gm-csf mRNA in an α-syn overexpressed Sprague-Dawley rat model. FIG. 8A provides representative images of TH+/Nissl+ dopaminergic neurons within the substantia nigra (column 1 and 2) of Sprague-Dawley rats that were stereotaxically-injected on the ipsilateral side with either PBS (Sham), an AAV control (AAV-GFP), AAV-α-syn alone, or AAV-α-syn followed by treatment with two different doses of Gm-csf mRNA, 0.01 mg/kg or 0.1 mg/kg (scale bar, 1000 μm). Representative images of TH+ dopaminergic neuron termini within the striatum after treatment are displayed in column 3 (scale bar, 1000 μm). FIG. 8B provides a graph depicting stereological quantification of the ipsilateral/contralateral ratios of total numbers of surviving dopaminergic (TH+/Nissl+, black bar) and non-dopaminergic (TH−/Nissl+, grey bar) neurons within the ipsilateral and contralateral hemispheres of the SN following α-syn overexpression. Differences in means (±SEM, n=7) were determined where p<0.05 compared with Sham (indicated by the letter “a” in drawings), AAV-GFP (indicated by the letter “b” in drawings), or AAV-α-syn treatment (indicated by the letter “c” in drawings). FIG. 8C provides a graph depicting ipsilateral/contralateral ratios of striatal TH dopaminergic termini density within ipsilateral and contralateral hemispheres of the striatum. Differences in means (±SEM, n=7) were determined where P<0.05 compared with Sham (indicated by the letter “a” in drawings), AAV-GFP (indicated by the letter “b” in drawings), AAV-α-syn (indicated by the letter “c” in drawings), or AAV-α-syn+0.01 mg/kg Gm-csf mRNA (indicated by the letter “d” in drawings). FIG. 8D provides representative images of Iba1+ microglia within the substantia nigra on both contralateral and ipsilateral sides (scale bar, 40 μm). FIG. 8E provides a graph depicting the quantification of reactive, amoeboid Iba1+ microglia density utilizing stereological analysis displayed as a ratio of ipsilateral and contralateral densities. Differences in means (±SEM, n=7) were determined where p<0.05 compared with Sham (indicated by the letter “a” in drawings), AAV-GFP (indicated by the letter “b” in drawings), or AAV-α-syn (indicated by the letter “c” in drawings).

FIGS. 9A-9D depict peripheral cytokine profiles following treatment with AAV alpha-syn and LNP formulated Gm-csf mRNA, or treatment with LNP formulated Gm-csf mRNA alone. FIG. 9A provides a graph depicting the fold change of CINC-1, CINC-2a/b, CINC-3, CNTF, Fractalkine, GMCSF, siCAM-1, and IP-10 peripheral cytokines within peripheral blood plasma following treatment with AAV-α-syn and 0.01 mg/kg Gm-csf mRNA or 0.1 mg/kg Gm-csf mRNA. FIG. 9B provides a graph depicting the fold change of IFNγ, IL-1a, IL-1B, IL-1ra, IL-2, IL-3, and IL-4. FIG. 9C provides a graph depicting the fold change of LIX, L-Selectin, MIG, MIP-1a, RANTES, CXCL7, and TIMP-1. FIG. 9D provides a graph depicting the fold change of IL-6, IL-10, IL-13, IL-17, TNFα, and VEGFA. Differences in means (±SEM, n=4) were determined where p<0.05 compared with AAV-α-syn (indicated by the letter “a” in drawings).

FIGS. 10-12 demonstrate the effects of intramuscular treatment with LNP formulated MSA-conjugated Gm-csf mRNA. FIG. 10 provides a graph depicting the quantification of plasma GM-CSF protein levels in peripheral blood 6 hours, and 1, 3, and 5 days after treatment with multiple ascending doses of LNP formulated MSA-conjugated Gm-csf mRNA scaffold, control NTFIX, or GM-CSF protein. FIG. 11 provides a graph depicting quantification of organ weight 1, 3, and 5 days after initial treatment. FIG. 12 provides graphs depicting absolute counts of white blood cells (WBC) (top left), monocytes (bottom left), neutrophils (bottom right), and lymphocytes (top right) within whole blood following treatment.

FIGS. 13-14 demonstrates the immunohistochemistry effects in mice administered increasing doses of LNP formulated MSA-conjugated Gm-csfmRNA at 1, 3, and 5 days after treatment. FIG. 13 provides graphs depicting quantification of CD3+ (top right), CD8+ (bottom right), CD4+ (bottom left), and CD4+CD25+FOXP3+ (top left) cells in peripheral blood following treatment with ascending doses of MSA-conjugated Gm-csfmRNA compared to treatment with native recombinant GM-CSF protein and control NTFIX. FIG. 14 provides graphs depicting assessment of Treg-mediated inhibition (±SEM) of CFSE-stained Tresps (CD4+CD25−) stimulated with anti-CD3/CD28 beads. Tregs were isolated from untreated (0 mg/kg), MSA-conjugated Gm-csf mRNA-treated (0.001 mg/kg-0.1 mg/kg), or recombinant GM-CSF protein-treated mice 1, 3, and 5 days after treatment.

FIGS. 15-17 show attenuation of MPTP-induced nigrostriatal neurodegeneration and microglial activation in LNP formulated MSA-conjugated Gm-csf mRNA treated animals. FIG. 15 provides photomicrographs of dopaminergic (TH+/Nissl+) neurons within the substantia nigra (SN) of mice, along with a graph depicting stereological quantification of total numbers of surviving TH+/Nissl+ and non-dopaminergic (TH−/Nissl+) neurons within the SN following MPTP intoxication. Differences in means (±SEM, n=15) were determined where p<0.05 compared with: groups treated with PBS (indicated by the letter “a” in drawings); groups treated with MPTP (indicated by the letter “b” in drawings); groups treated with 0.01 mg/kg LNP formulated MSA-conjugated Gm-csf mRNA (indicated by the letter “c” in drawings); groups treated with 0.03 mg/kg LNP formulated MSA-conjugated Gm-csf mRNA (indicated by the letter “d” in drawings), and groups treated with 0.1 mg/kg LNP formulated Gm-csf mRNA (indicated by the letter “e” in the drawings). FIG. 16 provides photomicrographs of dopaminergic (TH+/Nissl+) neurons within the within the striatum (STR) of mice, along with a graph depicting relative fold change of TH density in striatal dopaminergic termini normalized to PBS controls. Differences in means (±SEM, n=15) were determined where p<0.05 compared with PBS (indicated by the letter “a” in drawings); groups treated with MPTP (indicated by the letter “b” in drawings); groups treated with 0.01 mg/kg LNP formulated MSA-conjugated Gm-csf mRNA (indicated by the letter “c” in drawings); groups treated with 0.03 mg/kg LNP formulated MSA-conjugated Gm-csf mRNA (indicated by the letter “d” in drawings), and groups treated with 0.1 mg/kg LNP formulated Gm-csf mRNA (indicated by the letter “e” in the drawings). FIG. 17 provides photomicrographs of Mac-1+ microglia in the SN. For all images, mice were treated with PBS, MPTP, 0.01 mg/kg MSA-conjugated Gm-csfmRNA, 0.03 mg/kg MSA-conjugated Gm-csf mRNA, 0.1 mg/kg MSA-conjugated Gm-csf mRNA, or 0.1 mg/kg NTFIX. FIG. 17 also provides a graph depicting the quantification of reactive microglia taken from midbrains two days post MPTP-intoxication. Differences in means (±SEM, n=15) were determined where p<0.05 compared with PBS (indicated by the letter “a” in drawings); groups treated with MPTP (indicated by the letter “b” in drawings); groups treated with 0.01 mg/kg LNP formulated MSA-conjugated Gm-csf mRNA (indicated by the letter “c” in drawings); groups treated with 0.03 mg/kg LNP formulated MSA-conjugated Gm-csf mRNA (indicated by the letter “d” in drawings), and groups treated with 0.1 mg/kg LNP formulated Gm-csf mRNA (indicated by the letter “e” in the drawings). FIG. 18 provides photomicrographs of dopaminergic (TH+/Nissl+) neurons within the substantia nigra (SN) of mice, along with a graph depicting stereological quantification of total numbers of surviving TH+/Nissl+ and non-dopaminergic (TH−/Nissl+) neurons within the SN following MPTP intoxication for groups treated with PBS; MPTP; 0.1 mg/kg recombinant GM-CSF protein; and 0. 1 mg/kg LNP formulated MSA-conjugated GM-CSF mRNA. FIG. 19 provides photomicrographs of dopaminergic (TH+/Nissl+) neurons within the within the striatum (STR) of mice, along with a graph depicting relative fold change of TH density in striatal dopaminergic termini for groups treated with PBS; MPTP; 0.1 mg/kg recombinant GM-CSF protein; and 0.1 mg/kg LNP formulated MSA-conjugated Gm-csf mRNA.

FIGS. 20-27 show the results of LNP formulated RSA-conjugated Gm-csf mRNA treatment in α-syn overexpressed Sprague-Dawley rats. FIG. 20 provides a graph depicting quantification of Treg (CD4+CD25+)-mediated cell suppression (±SEM) at various Tresp:Treg ratios ex vivo. Treg-mediated suppression is expressed as percent inhibition. FIGS. 21-22 provide graphs depicting flow cytometric analysis of T cell phenotype frequencies in harvested spleens following treatment including CD3+, CD4+, CD4+CD25+FOXP3+, CD8+, and CD45R+ subsets. FIGS. 23-27 provide graphs depicting flow cytometric analysis of T cell phenotype frequencies including CD3+ (FIG. 23), CD4+ (FIG. 24), CD4+CD25+FOXP3+ (FIG. 25), CD8+ (FIG. 26), and CD45R+ (FIG. 27) subsets in peripheral blood following treatment.

### DETAILED DESCRIPTION

Regulatory T cells (also known as T regulatory cells or T regs) are of potential therapeutic value for prevention and/or treatment of Parkinson's disease (PD). In Parkinson's disease, disease onset and progression are often linked to diminished numbers of Tregs and their anti-proliferation and anti-inflammatory activities. However, the mechanism by which this occurs remains under investigation.

The therapeutic potential of GM-CSF (LEUKINE®, sargramostim) was recently disclosed in a Phase I clinical trial in PD patients (Gendelman, H. E., et al. (2017) NPJ Parkinsons Dis 3, 10). Daily administration of GM-CSF for 2 months led to increased Treg number and function. However, while daily treatment was generally well-tolerated, mild-to-moderate adverse events were experienced by all subjects including injection site reactions, elevated white blood cell counts, and bone pain. Furthermore, the limited bioavailability and short half-life of recombinant GM-CSF requires relatively high and frequent doses. Thus, there is a need to develop alternative delivery strategies for GM-CSF.

Accordingly, disclosed herein is a lipid nanoparticle (LNP) comprising a polynucleotide encoding a human GM-CSF polypeptide for use in the treatment of a subject having PD. As demonstrated in Examples 2-8, administration of LNP formulated GM-CSF mRNA resulted in, e.g., enhancement of Treg numbers, function, and superior neuroprotective activities in divergent models of human disease indicating an advance over the native GM-CSF protein.

In an aspect, the present disclosure provides, inter alia, methods of using a lipid nanoparticle (LNP) composition comprising a human granulocyte macrophage colony stimulating factor (GM-CSF) polypeptide. The LNP compositions of the present disclosure for use described herein comprise mRNA therapeutics encoding a human GM-CSF polypeptide. In an aspect, the LNP compositions of the present disclosure can be used in the treatment of Parkinson's disease in a subject.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 0%, 19% 18%, 15%, 14%, 13%, 12%, %11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein. Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” or “modification” refers to a changed state or a change in composition or structure of a molecule (e.g., polynucleotide, e.g., mRNA). Molecules (e.g., polynucleotides) may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof). In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 μm, or about 100 nm. Also routinely, nanoparticles have any one structural feature on a scale of from about 50 nm to about 500 nm, from about 50 nm to about 200 nm or from about 70 nm to about 120 nm. In exemplary embodiments, a nanoparticle is a particle having one or more dimensions of the order of about 1 nm to about 1000 nm. In other exemplary embodiments, a nanoparticle is a particle having one or more dimensions of the order of about 10 nm to about 500 nm. In other exemplary embodiments, a nanoparticle is a particle having one or more dimensions of the order of about 50 nm to about 200 nm. A spherical nanoparticle would have a diameter, for example, of between about 50 nm to about 100 nm or about 70 nm to about 120 nm. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (elF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

### LNPs Comprising a Polynucleotide Encoding GM-CSF for Use in Treating Parkinson's Disease

Granulocyte-macrophage colony stimulating factor (GM-CSF) is a cytokine which is secreted by many cells including, macrophages, T cells, mast cells, natural killer cells, endothelial cells and fibroblasts. GM-CSF is also known as colony stimulating factor 2 (CSF2). GM-CSF can stimulate stem cells to produce granulocytes (e.g., neutrophils) and monocytes, which can mature into macrophages and dendritic cells (DCs). GM-CSF can also increase DC maturation, function and recruitment.

In an aspect, the disclosure provides an LNP composition comprising a polynucleotide (e.g., mRNA) encoding a human GM-CSF polypeptide, e.g., as described herein, for use in the treatment of Parkinson's disease. In an embodiment, the LNP composition comprises an mRNA encoding a human GM-CSF polypeptide.

In an embodiment, the human GM-CSF polypeptide comprises an amino acid sequence having 100% identity to the amino acid sequence of a human GM-CSF polypeptide provided in Table 1A or 4A. In an embodiment, the human GM-CSF polypeptide comprises the amino acid sequence of SEQ ID NO: 1. In an embodiment, the human GM-CSF polypeptide comprises the amino acid sequence of SEQ ID NO: 8. In an embodiment, the human GM-CSF polypeptide comprises the amino acid sequence of SEQ ID NO: 8 without the leader sequence. In an embodiment, the human GM-CSF polypeptide comprises the amino acid sequence of SEQ ID NO: 187. In an embodiment, the human GM-CSF polypeptide comprises the amino acid sequence of SEQ ID NO: 187 without the leader sequence.

In an embodiment, the polynucleotide (e.g., mRNA) encoding the human GM-CSF polypeptide comprises a nucleotide sequence provided in Table 1A or 4A, or a nucleotide sequence having at least 85%, 90%, 95%, 96%, 97%, 98%, 99% or 100% identity to a nucleotide sequence provided in Table 1A or 4A. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 2. In an embodiment, the polynucleotide (e.g., mRNA) encoding the human GM-CSF polypeptide comprises the nucleotide sequence of SEQ ID NO: 2. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 3. In an embodiment, the polynucleotide (e.g., mRNA) encoding the human GM-CSF polypeptide comprises the nucleotide sequence of SEQ ID NO: 3. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 4. In an embodiment, the polynucleotide (e.g., mRNA) encoding the human GM-CSF polypeptide comprises the nucleotide sequence of SEQ ID NO: 4. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 5. In an embodiment, the polynucleotide (e.g., mRNA) encoding the human GM-CSF polypeptide comprises the nucleotide sequence of SEQ ID NO: 5. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 6. In an embodiment, the polynucleotide (e.g., mRNA) encoding the human GM-CSF polypeptide comprises the nucleotide sequence of SEQ ID NO: 6. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 7. In an embodiment, the polynucleotide (e.g., mRNA) encoding the human GM-CSF polypeptide comprises the nucleotide sequence of SEQ ID NO: 7. In an embodiment, the polynucleotide (e.g., mRNA) encoding the human GM-CSF polypeptide comprises a nucleotide sequence having at least 85%, 90%, 95%, 96%, 97%, 98%, 99% or 100% identity to the sequence of SEQ ID NO: 188. In an embodiment, the polynucleotide (e.g., mRNA) encoding the human GM-CSF polypeptide comprises the nucleotide sequence of SEQ ID NO: 188.

In an aspect, the disclosure provides an LNP composition comprising a polynucleotide (e.g., mRNA) encoding a murine GM-CSF polypeptide, e.g., as described herein, for use in the treatment of Parkinson's disease. In an embodiment, the LNP composition comprises an mRNA encoding a murine GM-CSF polypeptide.

In an embodiment, the murine GM-CSF polypeptide comprises an amino acid sequence having 100% identity to the amino acid sequence of a murine GM-CSF polypeptide provided in Table 1A. In an embodiment, the murine GM-CSF polypeptide comprises the amino acid sequence of SEQ ID NO: 9. In an embodiment, the murine GM-CSF polypeptide comprises the amino acid sequence of SEQ ID NO: 9 without the leader sequence.

In an embodiment, the polynucleotide (e.g., mRNA) encoding the murine GM-CSF polypeptide comprises a nucleotide sequence provided in Table 1A, or a nucleotide sequence having at least 85%, 90%, 95%, 96%, 97%, 98%, 99% or 100% identity to a nucleotide sequence provided in Table 1A. In an embodiment, the polynucleotide (e.g., mRNA) encoding the murine GM-CSF polypeptide comprises a nucleotide sequence having at least 85%, 90%, 95%, 96%, 97%, 98%, 99% or 100% identity to the sequence of SEQ ID NO: 4. In an embodiment, the polynucleotide (e.g., mRNA) encoding the murine GM-CSF polypeptide comprises the nucleotide sequence of SEQ ID NO: 4. In an embodiment, the polynucleotide (e.g., mRNA) encoding the murine GM-CSF polypeptide comprises a nucleotide sequence having at least 85%, 90%, 95%, 96%, 97%, 98%, 99% or 100% identity to the sequence of SEQ ID NO: 5. In an embodiment, the polynucleotide (e.g., mRNA) encoding the murine GM-CSF polypeptide comprises the nucleotide sequence of SEQ ID NO: 5.

In an aspect, the disclosure provides an LNP composition comprising a polynucleotide (e.g., mRNA) encoding a rat GM-CSF polypeptide, e.g., as described herein, for use in the treatment of Parkinson's disease. In an embodiment, the LNP composition comprises an mRNA encoding a rat GM-CSF polypeptide.

In an embodiment, the rat GM-CSF polypeptide comprises an amino acid sequence having 100% identity to the amino acid sequence of a rat GM-CSF polypeptide provided in Table 1A or 4A. In an embodiment, the rat GM-CSF polypeptide comprises the amino acid sequence of SEQ ID NO: 10. In an embodiment, the rat GM-CSF polypeptide comprises the amino acid sequence of SEQ ID NO: 10 without the leader sequence.

In an embodiment, the polynucleotide (e.g., mRNA) encoding the rat GM-CSF polypeptide comprises a nucleotide sequence provided in Table 1A or 4A, or a nucleotide sequence having at least 85%, 90%, 95%, 96%, 97%, 98%, 99% or 100% identity to a nucleotide sequence provided in Table 1A or 4A. In an embodiment, the polynucleotide (e.g., mRNA) encoding the rat GM-CSF polypeptide comprises a nucleotide sequence having at least 85%, 90%, 95%, 96%, 97%, 98%, 99% or 100% identity to the sequence of SEQ ID NO: 6. In an embodiment, the polynucleotide (e.g., mRNA) encoding the rat GM-CSF polypeptide comprises the nucleotide sequence of SEQ ID NO: 6. In an embodiment, the polynucleotide (e.g., mRNA) encoding the rat GM-CSF polypeptide comprises a nucleotide sequence having at least 85%, 90%, 95%, 96%, 97%, 98%, 99% or 100% identity to the sequence of SEQ ID NO: 7. In an embodiment, the polynucleotide (e.g., mRNA) encoding the rat GM-CSF polypeptide comprises the nucleotide sequence of SEQ ID NO: 7.

In an aspect, the disclosure provides an LNP composition comprising a polynucleotide (e.g., mRNA) encoding a cyno GM-CSF polypeptide, e.g., as described herein, for use in the treatment of Parkinson's disease. In an embodiment, the LNP composition comprises an mRNA encoding a cyno GM-CSF polypeptide.

In an embodiment, the cyno GM-CSF polypeptide comprises an amino acid sequence having 100% identity to the amino acid sequence of a cyno GM-CSF polypeptide provided in Table 1A. In an embodiment, the cyno GM-CSF polypeptide comprises the amino acid sequence of SEQ ID NO: 11. In an embodiment, the cyno GM-CSF polypeptide comprises the amino acid sequence of SEQ ID NO: 11 without the leader sequence.

In an embodiment, the polynucleotide (e.g., mRNA) encoding the cyno GM-CSF polypeptide comprises a nucleotide sequence provided in Table 1A, or a nucleotide sequence having at least 85%, 90%, 95%, 96%, 97%, 98%, 99% or 100% identity to a nucleotide sequence provided in Table 1A. In an embodiment, the polynucleotide (e.g., mRNA) encoding the cyno GM-CSF polypeptide comprises a nucleotide sequence having at least 85%, 90%, 95%, 96%, 97%, 98%, 99% or 100% identity to the sequence of SEQ ID NO: 12. In an embodiment, the polynucleotide (e.g., mRNA) encoding the cyno GM-CSF polypeptide comprises the nucleotide sequence of SEQ ID NO: 12.

In an embodiment, the GM-CSF molecule further comprises a half-life extender, e.g., a protein (or fragment thereof) that binds to a serum protein such as albumin, IgG, FcRn or transferrin. In an embodiment, the half-life extender comprises albumin or a fragment thereof, or an Fc domain of an antibody molecule (e.g., an Fc domain with enhanced FcRn binding). In an embodiment, the half-life extender is albumin, or a fragment thereof. In an embodiment, the half-life extender is albumin, e.g., human serum albumin (HSA), mouse serum albumin (MSA), cyno serum albumin (CSA) or rat serum albumin (RSA). In an embodiment, the half-life extender is human serum albumin (HSA). In an embodiment, the half-life extender is mouse serum albumin (MSA). In an embodiment, the half-life extender is cyno serum albumin (CSA). In an embodiment, the half-life extender is rat serum albumin (RSA). In a preferred embodiment, the species of the serum albumin molecule is the same as the species to be treated.

In an embodiment, the half-life extender is human serum albumin (HSA). In an embodiment, HSA comprises an amino acid sequence having at least 85%, 90%, 95%, 96%, 97%, 98%, 99% or 100% identity to the amino acid sequence of SEQ ID NO: 189. In an embodiment, HSA comprises the amino acid sequence of SEQ ID NO: 189.

In an embodiment, the LNP comprises a polynucleotide encoding a GM-CSF molecule comprising a half-life extender. In an embodiment, the half-life extender is human serum albumin (HSA). In an embodiment, the GM-CSF molecule comprising HSA, e.g., HSA-GM-CSF, comprises an amino acid sequence having at least 85%, 90%, 95%, 96%, 97%, 98%, 99% or 100% identity to an HSA-GM-CSF sequence provided in Table 1A or 4A. In an embodiment, the GM-CSF molecule comprising HSA, e.g., HSA-GM-CSF, comprises the amino acid sequence of an HSA-GM-CSF sequence provided in Table 1A or 4A. In an embodiment, the half-life extender is human serum albumin (HSA). In an embodiment, the GM-CSF molecule comprising HSA, e.g., HSA-GM-CSF, comprises an amino acid sequence having at least 85%, 90%, 95%, 96%, 97%, 98%, 99% or 100% identity to SEQ ID NO: 187. In an embodiment, the GM-CSF molecule comprising HSA, e.g., HSA-GM-CSF, comprises the amino acid sequence of SEQ ID NO: 187.

In an embodiment, an LNP composition comprising a second polynucleotide (e.g., mRNA) encoding a GM-CSF molecule comprising a half-life extender comprises a nucleotide sequence having at least 85%, 90%, 95%, 96%, 97%, 98%, 99% or 100% identity to the nucleic acid sequence of SEQ ID NO: 188. In an embodiment, the second polynucleotide encoding the GM-CSF molecule comprising a half-life extender comprises the nucleotide sequence of SEQ ID NO: 188.

In an embodiment, the polynucleotide (e.g., mRNA) encoding the GM-CSF molecule further comprises one or more elements, e.g., a 5′ UTR and/or a 3′ UTR disclosed herein, e.g., in Table 4B. In an embodiment, the 5′ UTR and/or 3′UTR comprise one or more micro RNA (miR) binding sites, e.g., as disclosed herein. Exemplary 5′ UTRs and 3′ UTRs are disclosed in the section entitled “5′ UTR and 3′UTR″ herein.

TABLE 1A
Exemplary GM-CSF sequences for use in treating Parkinson's disease

| SEQ | | |
|---|---|---|
| ID | Sequence | |
| NO | information | Sequence |
| 1 | Human GMCSF | APARSPSPSTQPWEHVNAIQEARRLLNLSRDTAAEMNETVEV |
| | polypeptide | ISEMFDLQEPTCLQTRLELYKQGLRGSLTKLKGPLTMMASH |
| | | YKQHCPPTPETSCATQIITFESFKENLKDFLLVIPFDCWEPVQE |
| 8 | Human GMCSF | MWLQSLLLLGTVACSISAPARSPSPSTQPWEHVNAIQEARRL |
| | Polypeptide | LNLSRDTAAEMNETVEVISEMFDLQEPTCLQTRLELYKQGLR |
| | with leader | GSLTKLKGPLTMMASHYKQHCPPTPETSCATQIITFESFKENL |
| | sequence | KDFLL VIPFDCWEPVQE |
| | (underlined) | |
| 2 | Human GMCSF | AUGUGGCUGCAGAGCCUGCUGCUCUUGGGCACUGUGGCC |
| | mRNA | UGCAGCAUCUCUGCACCCGCCCGCUCGCCCAGCCCCAGC |
| | sequence | ACGCAGCCCUGGGAGCAUGUGAAUGCCAUCCAGGAGGCC |
| | | CGGCGUCUCCUGAACCUGAGUAGAGACACUGCUGCUGAG |
| | | AUGAAUGAAACAGUAGAAGUCAUCUCAGAAAUGUUUGA |
| | | CCUCCAGGAGCCGACCUGCCUACAGACCCGCCUGGAGCU |
| | | GUACAAGCAGGGCCUGCGGGGCAGCCUCACCAAGCUCAA |
| | | GGGCCCCUUGACCAUGAUGGCCAGCCACUACAAGCAGCA |
| | | CUGCCCUCCAACCCCGGAAACUUCCUGUGCAACCCAGAU |
| | | UAUCACCUUUGAAAGUUUCAAAGAGAACCUGAAGGACU |
| | | UUCUGCUUGUCAUCCCCUUUGACUGCUGGGAGCCAGUCC |
| | | AGGAG |
| 3 | Human GMCSF | AGUACACAGA GAGAAAGGCU AAAGUUCUCU |
| | mRNA | GGAGGAUGUG GCUGCAGAGC CUGCUGCUCU |
| | sequence-2 | UGGGCACUGU GGCCUGCAGC AUCUCUGCAC |
| | | CCGCCCGCUC GCCCAGCCCC AGCACGCAGC |
| | | CCUGGGAGCA UGUGAAUGCC AUCCAGGAGG |
| | | CCCGGCGUCU CCUGAACCUG AGUAGAGACA |
| | | CUGCUGCUGA GAUGAAUGAA ACAGUAGAAG |
| | | UCAUCUCAGA AAUGUUUGAC CUCCAGGAGC |
| | | CGACCUGCCU ACAGACCCGC CUGGAGCUGU |
| | | ACAAGCAGGG CCUGCGGGGC AGCCUCACCA |
| | | AGCUCAAGGG CCCCUUGACC AUGAUGGCCA |
| | | GCCACUACAA GCAGCACUGC CCUCCAACCC |
| | | CGGAAACUUC CUGUGCAACC CAGAUUAUCA |
| | | CCUUUGAAAG UUUCAAAGAG AACCUGAAGG |
| | | ACUUUCUGCU UGUCAUCCCC UUUGACUGCU |
| | | GGGAGCCAGU CCAGGAGUGA GACCGGCCAG |
| | | AUGAGGCUGG CCAAGCCGGG GAGCUGCUCU |
| | | CUCAUGAAAC AAGAGCUAGA AACUCAGGAU |
| | | GGUCAUCUUG GAGGGACCAA GGGGUGGGCC |
| | | ACAGCCAUGG UGGGAGUGGC CUGGACCUGC |
| | | CCUGGGCCAC ACUGACCCUG AUACAGGCAU |
| | | GGCAGAAGAA UGGGAAUAUU UUAUACUGAC |
| | | AGAAAUCAGU AAUAUUUAUA UAUUUAUAUU |
| | | UUUAAAAUAU UUAUUUAUUU AUUUAUUUAA |
| | | GUUCAUAUUC CAUAUUUAUU CAAGAUGUUU |
| | | UACCGUAAUA AUUAUUAUUA AAAAUAUGCU |
| | | UCUACUUG |
| 9 | Murine GMCSF | MWLQNLLFLGIVVYSLSAPTRSPITVTRPWKHVEAIKEALNL |
| | Polypeptide | LDDMPVTLNEEVEVVSNEFSFKKLTCVQTRLKIFEQGLRGNF |
| | with leader | TKLKGALNMTASYYQTYCPPTPETDCETQVTTYADFIDSLKT |
| | sequence | FLTDIPFECKKPGQK |
| | (underlined) | |
| 4 | Murine GMCSF | AUGUGGCUUCAGAAUCUCUUGUUUCUUGGAAUCGUCGU |
| | mRNA | GUACAGCCUGUCAGCCCCAACUAGAUCGCCUAUCACUGU |
| | sequence | GACGCGCCCGUGGAAGCACGUGGAAGCCAUCAAGGAGGC |
| | | UCUGAAUCUGCUCGACGAUAUGCCAGUGACCCUGAACGA |
| | | GGAAGUCGAAGUGGUGUCCAACGAAUUUUCCUUCAAGA |
| | | AGUUGACCUGUGUUCAGACCCGGCUGAAGAUUUUCGAG |
| | | CAGGGCCUCAGGGGAAACUUCACCAAACUGAAGGGUGC |
| | | ACUGAACAUGACCGCCAGCUACUACCAGACCUAUUGCCC |
| | | UCCGACUCCGGAAACUGAUUGCGAGACUCAAGUCACCAC |
| | | CUACGCGGACUUCAUCGACUCGCUCAAGACGUUCCUGAC |
| | | UGACAUCCCCUUCGAGUGCAAGAAGCCGGGGCAGAAA |
| 5 | Murine GMCSF | GGUCAGACUG CCCAGGCAGG GUGGGAAAGG CCUUUAAAGC AGCCCGCAGG |
| | mRNA | UGGGCUGCCA GUUCUUGGAA GGGCUUAUUA AUGAAAACCC CCCAAGCCUG |
| | sequence-2 | ACAACCUGGG GGAAGGCUCA CUGGCCCCAU GUAUAGCUGA UAAGGGCCAG |
| | | GAGAUUCCAC AACUCAGGUA GUUCCCCCGC CCCCCUGGAG UUCUGUGGUC |
| | | ACCAUUAAUC AUUUCCUCUA ACUGUGUAUA UAAGAGCUCU UUUGCAGUGA |
| | | GCCCAGUACU CAGAGAGAAA GGCUAAGGUC CUGAGGAGGA UGUGGCUGCA |
| | | GAAUUUACUU UUCCUGGGCA UUGUGGUCUA CAGCCUCUCA GCACCCACCC |
| | | GCUCACCCAU CACUGUCACC CGGCCUUGGA AGCAUGUAGA GGCCAUCAAA |
| | | GAAGCCCUGA ACCUCCUGGA UGACAUGCCU GUCACGUUGA AUGAAGAGGU |
| | | AGAAGUCGUC UCUAACGAGU UCUCCUUCAA GAAGCUAACA UGUGUGCAGA |
| | | CCCGCCUGAA GAUAUUCGAG CAGGGUCUAC GGGGCAAUUU CACCAAACUC |
| | | AAGGGCGCCU UGAACAUGAC AGCCAGCUAC UACCAGACAU ACUGCCCCCC |
| | | AACUCCGGAA ACGGACUGUG AAACACAAGU UACCACCUAU GCGGAUUUCA |
| | | UAGACAGCCU UAAAACCUUU CUGACUGAUA UCCCCUUUGA AUGCAAAAAA |
| | | CCAGGCCAAA AAUGAGGAAG CCCAGGCCAG CUCUGAAUCC AGCUUCUCAG |
| | | ACUGCUGCUU UUGUGCCUGC GUAAUGAGCC AGGAACUUGG AAUUUCUGCC |
| | | UUAAAGGGAC CAAGAGAUGU GGCACAGCCA CAGUUGGAAG GCAGUAUAGC |
| | | CCUCUGAAAA CGCUGACUCA GCUUGGACAG CGGAAGACAA ACGAGAGAUA |
| | | UUUUCUACUG AUAGGGACCA UUAUAUUUAU UUAUAUAUUU AUAUUUUUUA |
| | | AAUAUUUAUU UAUUUAUUUA UUUAUUUUUG CAACUCUAUU UAUUGAGAAU |
| | | GUCUUACCAG AAUAAUAAAU UAUUAAAACU UUU |
| 10 | Rat Polypeptide | MWLQNLLFLGIVVYSFSAPTRSPNPVTRPWKHVDAIKEALSL |
| | with leader | LNDMRALENEKNEDVDIISNEFSIQRPTCVQTRLKLYKQGLR |
| | sequence | GNLTKLNGALTMIASHYQTNCPPTPETDCEIEVTTFEDFIKNL |
| | (underlined) | KGFLFDIPFDCWKPVQK |
| 6 | Rat GMCSF | AUGUGGCUGCAGAACCUGCUGUUCCUGGGCAUCGUGGU |
| | mRNA | GUACAGCUUCAGCGCCCCUACCAGAAGCCCUAACCCUGU |
| | sequence | GACCAGACCUUGGAAGCACGUGGACGCCAUCAAGGAGGC |
| | | CCUGAGCCUGCUGAACGACAUGAGAGCCCUGGAGAACGA |
| | | GAAGAACGAGGACGUGGACAUCAUCAGCAACGAGUUCA |
| | | GCAUCCAGAGACCUACCUGCGUGCAGACCAGACUGAAGC |
| | | UGUACAAGCAGGGCCUGAGAGGCAACCUGACCAAGCUG |
| | | AACGGCGCCCUGACCAUGAUCGCCAGCCACUACCAGACC |
| | | AACUGCCCUCCUACCCCUGAGACCGACUGCGAGAUCGAG |
| | | GUGACCACCUUCGAGGACUUCAUCAAGAACCUGAAGGGC |
| | | UUCCUGUUCGACAUCCCUUUCGACUGCUGGAAGCCUGUG |
| | | CAGAAG |
| 7 | Rat GMCSF | AUGUGGCUGC AGAAUUUACU UUUCCUGGGC AUUGUGGUCU ACAGUUUCUC |
| | mRNA | AGCACCCACC CGCUCGCCCA ACCCUGUCAC CCGGCCCUGG AAGCAUGUAG |
| | sequence-2 | AUGCCAUCAA AGAAGCUCUG AGCCUCCUAA AUGACAUGCG UGCUCUGGAG |
| | | AACGAAAAGA ACGAAGACGU AGACAUCAUC UCUAAUGAGU UCUCCAUCCA |
| | | GAGGCCGACA UGUGUGCAGA CCCGCCUGAA GCUAUACAAG CAGGGUCUAC |
| | | GGGGCAACCU CACCAAACUC AAUGGCGCCU UGACCAUGAU AGCCAGCCAC |
| | | UACCAGACGA ACUGCCCUCC AACCCCGGAA ACUGACUGUG AAAUAGAAGU |
| | | CACCACCUUU GAGGAUUUCA UAAAGAACCU UAAAGGCUUU CUGUUUGAUA |
| | | UCCCUUUUGA CUGCUGGAAG CCGGUCCAGA AAUGAGGAGG C |
| 11 | Cyno | MWLQGLLLLGTVACSISAPARSPSPGTQPWEHVNAIQEARRL |
| | Polypeptide | LNLSRDTAAEMNKTVEVVSEMFDLQEPSCLQTRLELYKQGL |
| | with leader | QGSLTKLKGPLTMMASHYKQHCPPTPETSCATQIITFQSFKE |
| | sequence | NLKDFLLVIPFDCWEPVQE |
| | (underlined) | |
| 12 | Cyno GMCSF | AUGUGGCUGCAGGGCCUGCUGCUGCUGGGCACCGUGGCC |
| | mRNA | UGCAGCAUCAGCGCCCCUGCCAGAAGCCCUAGCCCUGGC |
| | sequence | ACCCAGCCUUGGGAGCACGUGAACGCCAUCCAGGAGGCC |
| | | AGAAGACUGCUGAACCUGAGCAGAGACACCGCCGCCGAG |
| | | AUGAACAAGACCGUGGAGGUGGUGAGCGAGAUGUUCGA |
| | | CCUGCAGGAGCCUAGCUGCCUGCAGACCAGACUGGAGCU |
| | | GUACAAGCAGGGCCUGCAGGGCAGCCUGACCAAGCUGAA |
| | | GGGCCCUCUGACCAUGAUGGCCAGCCACUACAAGCAGCA |
| | | CUGCCCUCCUACCCCUGAGACCAGCUGCGCCACCCAGAU |
| | | CAUCACCUUCCAGAGCUUCAAGGAGAACCUGAAGGACUU |
| | | CCUGCUGGUGAUCCCUUUCGACUGCUGGGAGCCUGUGCA |
| | | GGAG |
| 187 | NVL(SdP) | MKWVTFISLLFLFSSAYSRGVFRRDAHKSEVAHRFKDLGE |
| | HSA- | ENFKALVLIAFAQYLQQCPFEDHVKLVNEVTEFAKTCVADES |
| | hsGMCSF | AENCDKSLHTLFGDKLCTVATLRETYGEMADCCAKQEPERN |
| | (underlined) | ECFLQHKDDNPNLPRLVRPEVDVMCTAFHDNEETFLKKYLY |
| | Note: leader | EIARRHPYFYAPELLFFAKRYKAAFTECCQAADKAACLLPKL |
| | sequence in | DELRDEGKASSAKQRLKCASLQKFGERAFKAWAVARLSQRF |
| | bold and | PKAEFAEVSKLVTDLTKVHTECCHGDLLECADDRADLAKYI |
| | underline; | CENQDSISSKLKECCEKPLLEKSHCIAEVENDEMPADLPSLAA |
| | linker italicized | DFVESKDVCKNYAEAKDVFLGMFLYEYARRHPDYSVVLLL |
| | and underlined | RLAKTYETTLEKCCAAADPHECYAKVFDEFKPLVEEPQNLIK |
| | | QNCELFEQLGEYKFQNALLVRYTKKVPQVSTPTLVEVSRNL |
| | | GKVGSKCCKHPEAKRMPCAEDYLSVVLNQLCVLHEKTPVSD |
| | | RVTKCCTESLVNRRPCFSALEVDETYVPKEFNAETFTFHADIC |
| | | TLSEKERQIKKQTALVELVKHKPKATKEQLKAVMDDFAAFV |
| | | EKCCKADDKETCFAEEGKKLVAASQAALGLGGGSAPARSPS |
| | | PSTQPWEHVNAIQEARRLLNLSRDTAAEMNETVEVISEMFDL |
| | | QEPTCLQTRLELYKQGLRGSLTKLKGPLTMMASHYKQHCPP |
| | | TPETSCATQIITFESFKENLKDFLLVIPFDCWEPVQE |
| 188 | NVL(SdP) | AUGAAGUGGGUGACCUUCAUCAGCCUGCUGUUCCUGUUCAGCAGCG |
| | HSA- | CCUACAGCAGAGGCGUGUUCAGAAGAGACGCCCACAAGAGCGAGGU |
| | hsGMCSF | GGCCCACAGAUUCAAGGACCUGGGCGAGGAGAACUUCAAGGCCCUG |
| | | GUGCUGAUCGCCUUCGCCCAGUACCUGCAGCAGUGCCCUUUCGAGGA |
| | | CCACGUGAAGCUGGUGAACGAGGUGACCGAGUUCGCCAAGACCUGC |
| | | GUGGCCGACGAGAGCGCCGAGAACUGCGACAAGAGCCUGCACACCCU |
| | | GUUCGGCGACAAGCUGUGCACCGUGGCCACCCUGAGAGAAACUUACG |
| | | GCGAGAUGGCCGACUGCUGCGCCAAGCAGGAGCCAGAGCGGAACGA |
| | | GUGCUUCCUGCAACACAAGGACGACAACCCUAACCUGCCUAGACUGG |
| | | UCCGGCCUGAGGUGGACGUGAUGUGCACGGCCUUCCACGACAACGAG |
| | | GAGACUUUCCUGAAGAAGUACCUGUACGAGAUCGCCAGAAGACACC |
| | | CUUACUUCUACGCCCCUGAGCUCCUGUUCUUCGCGAAGAGAUACAAG |
| | | GCCGCCUUCACCGAGUGCUGCCAGGCCGCCGACAAGGCAGCUUGCCU |
| | | GCUGCCUAAGCUGGACGAGCUGAGAGACGAGGGCAAGGCCUCCUCA |
| | | GCUAAGCAGAGACUGAAGUGCGCCAGCCUGCAGAAGUUCGGUGAGA |
| | | GAGCAUUCAAGGCUUGGGCCGUCGCAAGACUGUCACAGAGAUUCCC |
| | | UAAGGCAGAAUUCGCGGAGGUGAGCAAGCUAGUGACCGACCUGACC |
| | | AAGGUGCAUACAGAGUGCUGCCACGGCGACCUGCUGGAGUGCGCCG |
| | | ACGACAGAGCCGACCUGGCCAAGUACAUCUGCGAGAACCAGGACAGC |
| | | AUCAGCUCCAAGCUGAAGGAGUGCUGUGAGAAGCCUCUGCUGGAGA |
| | | AGUCACACUGCAUUGCCGAGGUCGAGAACGACGAGAUGCCUGCCGA |
| | | UCUUCCUAGCCUUGCCGCCGAUUUCGUGGAGAGCAAGGACGUGUGC |
| | | AAGAACUACGCCGAGGCAAAGGACGUGUUCCUGGGCAUGUUCCUUU |
| | | ACGAAUACGCUCGCCGGCAUCCAGACUACAGCGUGGUGCUGCUGCUG |
| | | AGAUUGGCCAAGACUUACGAGACGACCCUCGAGAAGUGUUGCGCAG |
| | | CAGCUGAUCCUCACGAGUGUUACGCCAAGGUGUUCGACGAGUUCAA |
| | | GCCGCUUGUGGAGGAGCCUCAGAACCUGAUCAAGCAGAAUUGUGAG |
| | | CUGUUCGAGCAGCUGGGUGAGUACAAGUUCCAGAACGCCCUGCUGG |
| | | UGCGCUACACCAAGAAGGUGCCUCAAGUGUCUACCCCUACCCUGGUU |
| | | GAAGUUUCCCGCAACCUGGGCAAGGUGGGCAGCAAGUGCUGCAAGC |
| | | AUCCUGAAGCAAAGAGGAUGCCUUGCGCCGAGGACUACCUGUCAGU |
| | | GGUCCUUAACCAGCUGUGCGUGCUGCACGAGAAGACCCCUGUGAGCG |
| | | ACAGAGUGACAAAGUGUUGUACCGAGAGCCUGGUCAACAGAAGACC |
| | | UUGCUUCAGCGCCCUGGAAGUCGACGAGACAUACGUGCCUAAGGAG |
| | | UUCAACGCCGAAACCUUCACCUUCCACGCCGACAUCUGCACACUGAG |
| | | CGAGAAGGAGAGACAGAUCAAGAAGCAGACCGCCCUGGUCGAGUUG |
| | | GUGAAGCACAAGCCUAAGGCCACCAAGGAGCAACUCAAGGCCGUGA |
| | | UGGACGACUUCGCGGCCUUCGUUGAGAAGUGCUGUAAGGCUGACGA |
| | | CAAGGAGACGUGCUUCGCUGAGGAGGGUAAGAAGCUUGUCGCCGCC |
| | | UCUCAGGCCGCUUUGGGACUCGGCGGCGGCAGUGCGCCUGCCAGAAG |
| | | CCCUUCCCCAUCUACCCAGCCUUGGGAGCACGUGAACGCCAUCCAGG |
| | | AGGCCAGACGUCUGCUGAACCUGUCACGGGAUACCGCAGCUGAGAU |
| | | GAACGAAACUGUUGAGGUCAUCAGCGAGAUGUUCGACCUACAGGAA |
| | | CCUACCUGCUUGCAGACCAGACUGGAGCUGUACAAGCAGGGAUUAA |
| | | GAGGCUCCCUGACGAAGCUUAAGGGCCCUCUGACCAUGAUGGCCAGC |
| | | CACUAUAAGCAGCACUGCCCUCCUACCCCUGAAACGUCGUGUGCUAC |
| | | CCAGAUCAUCACCUUCGAGAGCUUCAAGGAGAAUCUGAAGGACUUC |
| | | CUGCUCGUUAUUCCGUUCGAUUGUUGGGAGCCUGUGCAGGAG |
| 189 | Human serum | AHKSEVAHRFKDLGEENFKALVLIAFAQYLQQCPFEDHVKL |
| | albumin (HSA) | VNEVTEFAKTCVADESAENCDKSLHTLFGDKLCTVATLRET |
| | | YGEMADCCAKQEPERNECFLQHKDDNPNLPRLVRPEVDVM |
| | | CTAFHDNEETFLKKYLYEIARRHPYFYAPELLFFAKRYKAAF |
| | | TECCQAADKAACLLPKLDELRDEGKASSAKQRLKCASLQKF |
| | | GERAFKAWAVARLSQRFPKAEFAEVSKLVTDLTKVHTECCH |
| | | GDLLECADDRADLAKYICENQDSISSKLKECCEKPLLEKSHCI |
| | | AEVENDEMPADLPSLAADFVESKDVCKNYAEAKDVFLGMFL |
| | | YEYARRHPDYSVVLLLRLAKTYETTLEKCCAAADPHECYAK |
| | | VFDEFKPLVEEPQNLIKQNCELFEQLGEYKFQNALLVRYTKK |
| | | VPQVSTPTLVEVSRNLGKVGSKCCKHPEAKRMPCAEDYLSV |
| | | VLNQLCVLHEKTPVSDRVTKCCTESLVNRRPCFSALEVDETY |
| | | VPKEFNAETFTFHADICTLSEKERQIKKQTALVELVKHKPKAT |
| | | KEQLKAVMDDFAAFVEKCCKADDKETCFAEEGKKLVAASQ |
| | | AALGL |

Without wishing to be bound by theory, a skilled person would understand that in some embodiments the amino acid sequence of RGVFRRD can constitute part of the leader sequence described herein as HSA is generally made as a pre-pro-peptide.

In some embodiments, a polynucleotide of the present disclosure, for example a polynucleotide comprising an mRNA nucleotide sequence encoding a polypeptide, comprises (1) a 5′ cap, e.g., as disclosed herein, (2) a 5′ UTR, e.g., as provided in Table 4A, (3) a nucleotide sequence ORF provided in Table 1A, or 4A, (4) a stop codon, (5) a 3′UTR, e.g., as provided in Table 4A, and (6) a poly-A tail, e.g., as disclosed herein, e.g., a poly-A tail of about 100 residues, e.g., SEQ ID NO: 25.

In some embodiments, a polynucleotide comprising an mRNA nucleotide sequence encoding a GM-CSF polypeptide, comprises SEQ ID NO: 204 that consists from 5′ to 3′ end: 5′ UTR of SEQ ID NO: 202, ORF sequence of SEQ ID NO: 201, 3′ UTR of SEQ ID NO: 203 and Poly A tail of SEQ ID NO: 25.

In some embodiments, a polynucleotide comprising an mRNA nucleotide sequence encoding a GM-CSF polypeptide, comprises SEQ ID NO: 209 that consists from 5′ to 3′ end: 5′ UTR of SEQ ID NO: 207, ORF sequence of SEQ ID NO: 206, 3′ UTR of SEQ ID NO: 208 and Poly A tail of SEQ ID NO: 25.

In some embodiments, a polynucleotide comprising an mRNA nucleotide sequence encoding a GM-CSF polypeptide, comprises SEQ ID NO: 214 that consists from 5′ to 3′ end: 5′ UTR of SEQ ID NO: 212, ORF sequence of SEQ ID NO: 211, 3′ UTR of SEQ ID NO: 213 and Poly A tail of SEQ ID NO: 25.

In some embodiments, a polynucleotide comprising an mRNA nucleotide sequence encoding a GM-CSF polypeptide, comprises SEQ ID NO: 219 that consists from 5′ to 3′ end: 5′ UTR of SEQ ID NO: 217, ORF sequence of SEQ ID NO: 216, 3′ UTR of SEQ ID NO: 218 and Poly A tail of SEQ ID NO: 25.

In some embodiments, a polynucleotide comprising an mRNA nucleotide sequence encoding a GM-CSF polypeptide, comprises SEQ ID NO: 224 that consists from 5′ to 3′ end: 5′ UTR of SEQ ID NO: 222, ORF sequence of SEQ ID NO: 221, 3′ UTR of SEQ ID NO: 223 and Poly A tail of SEQ ID NO: 25.

TABLE 4A
Exemplary GM-CSF construct sequences for use in treating Parkinson's disease

| | | | | | | Con- |
|---|---|---|---|---|---|---|
| | mRNA | ORF Sequence | ORF Sequence | 5′ UTR | 3′ UTR | struct |
| | Name | (Amino Acid) | (Nucleotide) | Sequence | Sequence | Sequence |
| SEQ ID NO: | | 200 | 201 | 202 | 203 | 204 |
| | Cyno.GMC | MWLQGLLLLGTV | AUGUGGCUGCA | GGGAA | UGAUAA | SEQ ID |
| | SF | ACSISAPARSPSP | GGGCCUGCUGC | AUAAG | UAGGCU | NO: 204 |
| | G5 | GTQPWEHVNAIQ | UGCUGGGCACC | AGAGA | GGAGCC | consists |
| | Cap: C1 | EARRLLNLSRDT | GUGGCCUGCAG | AAAGA | UCGGUG | from 5′ to |
| | Poly A | AAEMNKTVEVVS | CAUCAGCGCCCC | AGAGU | GCCUAG | 3′ end: 5′ |
| | tail: 100 nt | EMFDLQEPSCLQ | UGCCAGAAGCCC | AAGAA | CUUCUU | UTR of |
| | | TRLELYKQGLQG | UAGCCCUGGCAC | GAAAU | GCCCCU | SEQ ID |
| | | SLTKLKGPLTMM | CCAGCCUUGGG | AUAAG | UGGGCC | NO: 202, |
| | | ASHYKQHCPPTP | AGCACGUGAAC | ACCCC | UCCCCC | ORF |
| | | ETSCATQIITFQSF | GCCAUCCAGGA | GGCGC | CAGCCC | sequence |
| | | KENLKDFLLVIPF | GGCCAGAAGAC | CGCCA | CUCCUC | of SEQ ID |
| | | DCWEPVQE | UGCUGAACCUG | CC | CCCUUC | NO: 201, |
| | | | AGCAGAGACAC | | CUGCAC | 3′ UTR of |
| | | | CGCCGCCGAGAU | | CCGUAC | SEQ ID |
| | | | GAACAAGACCG | | CCCCGU | NO: 203 |
| | | | UGGAGGUGGUG | | GGUCUU | and Poly |
| | | | AGCGAGAUGUU | | UGAAUA | A tail of |
| | | | CGACCUGCAGG | | AAGUCU | SEQ ID |
| | | | AGCCUAGCUGCC | | GAGUGG | NO: 25 |
| | | | UGCAGACCAGA | | GCGGC | |
| | | | CUGGAGCUGUA | | | |
| | | | CAAGCAGGGCC | | | |
| | | | UGCAGGGCAGC | | | |
| | | | CUGACCAAGCU | | | |
| | | | GAAGGGCCCUC | | | |
| | | | UGACCAUGAUG | | | |
| | | | GCCAGCCACUAC | | | |
| | | | AAGCAGCACUG | | | |
| | | | CCCUCCUACCCC | | | |
| | | | UGAGACCAGCU | | | |
| | | | GCGCCACCCAGA | | | |
| | | | UCAUCACCUUCC | | | |
| | | | AGAGCUUCAAG | | | |
| | | | GAGAACCUGAA | | | |
| | | | GGACUUCCUGC | | | |
| | | | UGGUGAUCCCU | | | |
| | | | UUCGACUGCUG | | | |
| | | | GGAGCCUGUGC | | | |
| | | | AGGAG | | | |
| SEQ ID NO: | | 205 | 206 | 207 | 208 | 209 |
| | Rn. GMCSF | MWLQNLLFLGIV | AUGUGGCUGCA | GGGAA | UGAUAA | SEQ ID |
| | G5 | VYSFSAPTRSPNP | GAACCUGCUGU | AUAAG | UAGGCU | NO: 209 |
| | Cap: C1 | VTRPWKHVDAIK | UCCUGGGCAUC | AGAGA | GGAGCC | consists |
| | Poly A | EALSLLNDMRAL | GUGGUGUACAG | AAAGA | UCGGUG | from 5′ to |
| | tail: 100 nt | ENEKNEDVDIISN | CUUCAGCGCCCC | AGAGU | GCCUAG | 3′ end: 5′ |
| | | EFSIQRPTCVQTR | UACCAGAAGCCC | AAGAA | CUUCUU | UTR of |
| | | LKLYKQGLRGNL | UAACCCUGUGA | GAAAU | GCCCCU | SEQ ID |
| | | TKLNGALTMIAS | CCAGACCUUGG | AUAAG | UGGGCC | NO: 207, |
| | | HYQTNCPPTPETD | AAGCACGUGGA | ACCCC | UCCCCC | ORF |
| | | CEIEVTTFEDFIKN | CGCCAUCAAGG | GGCGC | CAGCCC | sequence |
| | | LKGFLFDIPFDCW | AGGCCCUGAGCC | CGCCA | CUCCUC | of SEQ ID |
| | | KPVQK | UGCUGAACGAC | CC | CCCUUC | NO: 206, |
| | | | AUGAGAGCCCU | | CUGCAC | 3' UTR of |
| | | | GGAGAACGAGA | | CCGUAC | SEQ ID |
| | | | AGAACGAGGAC | | CCCCGU | NO: 208 |
| | | | GUGGACAUCAU | | GGUCUU | and Poly |
| | | | CAGCAACGAGU | | UGAAUA | A tail of |
| | | | UCAGCAUCCAG | | AAGUCU | SEQ ID |
| | | | AGACCUACCUGC | | GAGUGG | NO: 25 |
| | | | GUGCAGACCAG | | GCGGC | |
| | | | ACUGAAGCUGU | | | |
| | | | ACAAGCAGGGC | | | |
| | | | CUGAGAGGCAA | | | |
| | | | CCUGACCAAGCU | | | |
| | | | GAACGGCGCCCU | | | |
| | | | GACCAUGAUCG | | | |
| | | | CCAGCCACUACC | | | |
| | | | AGACCAACUGCC | | | |
| | | | CUCCUACCCCUG | | | |
| | | | AGACCGACUGC | | | |
| | | | GAGAUCGAGGU | | | |
| | | | GACCACCUUCGA | | | |
| | | | GGACUUCAUCA | | | |
| | | | AGAACCUGAAG | | | |
| | | | GGCUUCCUGUU | | | |
| | | | CGACAUCCCUUU | | | |
| | | | CGACUGCUGGA | | | |
| | | | AGCCUGUGCAG | | | |
| | | | AAG | | | |
| SEQ ID NO: | | 210 | 211 | 212 | 213 | 214 |
| | Mm.GMCSF | MWLQNLLFLGIV | AUGUGGCUUCA | GGGAA | GGGAAA | SEQ ID |
| | G5 | VYSLSAPTRSPIT | GAAUCUCUUGU | AUAAG | UAAGAG | NO: 214 |
| | Cap: C1 | VTRPWKHVEAIK | UUCUUGGAAUC | AGAGA | AGAAAA | consists |
| | Poly A | EALNLLDDMPVT | GUCGUGUACAG | AAAGA | GAAGAG | from 5′ to |
| | tail: 100 nt | LNEEVEVVSNEFS | CUUAUCAGCCCC | AGAGU | UAAGAA | 3′ end: 5′ |
| | | FKKLTCVQTRLKI | AACUAGAUCGC | AAGAA | GAAAUA | UTR of |
| | | FEQGLRGNFTKL | CUAUCACGGUG | GAAAU | UAAGAG | SEQ ID |
| | | KGALNMTASYY | ACGCGCCCGUGG | AUAAG | CCACC | NO: 212, |
| | | QTYCPPTPETDCE | AAGCACGUAGA | AGCCA | | ORF |
| | | TQVTTYADFIDSL | AGCCAUCAAGG | CC | | sequence |
| | | KTFLTDIPFECKK | AGGCUCUCAAU | | | of SEQ ID |
| | | PGQK | UUACUCGACGA | | | NO: 211, |
| | | | UAUGCCAGUGA | | | 3' UTR of |
| | | | CCCUUAACGAG | | | SEQ ID |
| | | | GAAGUCGAAGU | | | NO: 213 |
| | | | GGUGUCCAACG | | | and Poly |
| | | | AAUUUUCCUUC | | | A tail of |
| | | | AAGAAGUUGAC | | | SEQ ID |
| | | | CUGUGUUCAGA | | | NO: 25 |
| | | | CCCGGCUGAAG | | | |
| | | | AUUUUCGAGCA | | | |
| | | | GGGCCUCAGGG | | | |
| | | | GAAACUUCACC | | | |
| | | | AAACUGAAGGG | | | |
| | | | UGCACUGAACA | | | |
| | | | UGACCGCCAGCU | | | |
| | | | ACUACCAGACCU | | | |
| | | | AUUGCCCUCCGA | | | |
| | | | CUCCGGAAACU | | | |
| | | | GAUUGCGAGAC | | | |
| | | | UCAAGUCACCAC | | | |
| | | | CUACGCGGACU | | | |
| | | | UCAUCGACUCGC | | | |
| | | | UCAAGACGUUC | | | |
| | | | CUGACUGACAU | | | |
| | | | CCCCUUCGAGUG | | | |
| | | | CAAGAAGCCGG | | | |
| | | | GGCAGAAA | | | |
| SEQ ID NO: | | 215 | 216 | 217 | 218 | 219 |
| | hs. GMCSF | MWLQSLLLLGTV | AUGUGGCUGCA | GGGAA | UGAUAA | SEQ ID |
| | G5 | ACSISAPARSPSPS | GAGCCUGCUGC | AUAAG | UAGGCU | NO: 219 |
| | Cap: C1 | TQPWEHVNAIQE | UCUUGGGCACU | AGAGA | GGAGCC | consists |
| | Poly A | ARRLLNLSRDTA | GUGGCCUGCAG | AAAGA | UCGGUG | from 5′ to |
| | tail: 100 nt | AEMNETVEVISE | CAUCUCUGCACC | AGAGU | GCCUAG | 3′ end: 5′ |
| | | MFDLQEPTCLQT | CGCCCGCUCGCC | AAGAA | CUUCUU | UTR of |
| | | RLELYKQGLRGS | CAGCCCCAGCAC | GAAAU | GCCCCU | SEQ ID |
| | | LTKLKGPLTMMA | GCAGCCCUGGG | AUAAG | UGGGCC | NO: 217, |
| | | SHYKQHCPPTPET | AGCAUGUGAAU | ACCCC | UCCCCC | ORF |
| | | SCATQIITFESFKE | GCCAUCCAGGA | GGCGC | CAGCCC | sequence |
| | | NLKDFLLVIPFDC | GGCCCGGCGUCU | CGCCA | CUCCUC | of SEQ ID |
| | | WEPVQE | CCUGAACCUGA | CC | CCCUUC | NO: 216, |
| | | | GUAGAGACACU | | CUGCAC | 3′ UTR of |
| | | | GCUGCUGAGAU | | CCGUAC | SEQ ID |
| | | | GAAUGAAACAG | | CCCCGU | NO: 218 |
| | | | UAGAAGUCAUC | | GGUCUU | and Poly |
| | | | UCAGAAAUGUU | | UGAAUA | A tail of |
| | | | UGACCUCCAGG | | AAGUCU | SEQ ID |
| | | | AGCCGACCUGCC | | GAGUGG | NO: 25 |
| | | | UACAGACCCGCC | | GCGGC | |
| | | | UGGAGCUGUAC | | | |
| | | | AAGCAGGGCCU | | | |
| | | | GCGGGGCAGCC | | | |
| | | | UCACCAAGCUCA | | | |
| | | | AGGGCCCCUUG | | | |
| | | | ACCAUGAUGGC | | | |
| | | | CAGCCACUACAA | | | |
| | | | GCAGCACUGCCC | | | |
| | | | UCCAACCCCGGA | | | |
| | | | AACUUCCUGUG | | | |
| | | | CAACCCAGAUU | | | |
| | | | AUCACCUUUGA | | | |
| | | | AAGUUUCAAAG | | | |
| | | | AGAACCUGAAG | | | |
| | | | GACUUUCUGCU | | | |
| | | | UGUCAUCCCCUU | | | |
| | | | UGACUGCUGGG | | | |
| | | | AGCCAGUCCAG | | | |
| | | | GAG | | | |
| SEQ ID NO: | | 220 | 221 | 222 | 223 | 224 |
| | NVL(SdP)_ | MKWVTFISLLFLF | AUGAAGUGGGU | GGGAA | UGAUAA | SEQ ID |
| | HSA- | SSAYSRGVFRRD | GACCUUCAUCA | AUAAG | UAGGCU | NO: 224 |
| | hsGMCSF | AHKSEVAHRFKD | GCCUGCUGUUCC | AGAGA | GGAGCC | consists |
| | G5 | LGEENFKALVLIA | UGUUCAGCAGC | AAAGA | UCGGUG | from 5′ to |
| | Cap: C1 | FAQYLQQCPFED | GCCUACAGCAG | AGAGU | GCCUAG | 3′ end: 5′ |
| | Poly A | HVKLVNEVTEFA | AGGCGUGUUCA | AAGAA | CUUCUU | UTR of |
| | tail: 100 nt | KTCVADESAENC | GAAGAGACGCC | GAAAU | GCCCCU | SEQ ID |
| | | DKSLHTLFGDKL | CACAAGAGCGA | AUAAG | UGGGCC | NO: 222, |
| | | CTVATLRETYGE | GGUGGCCCACA | ACCCC | UCCCCC | ORF |
| | | MADCCAKQEPER | GAUUCAAGGAC | GGCGC | CAGCCC | sequence |
| | | NECFLQHKDDNP | CUGGGCGAGGA | CGCCA | CUCCUC | of SEQ ID |
| | | NLPRLVRPEVDV | GAACUUCAAGG | CC | CCCUUC | NO: 221, |
| | | MCTAFHDNEETF | CCCUGGUGCUG | | CUGCAC | 3′ UTR of |
| | | LKKYLYEIARRHP | AUCGCCUUCGCC | | CCGUAC | SEQ ID |
| | | YFYAPELLFFAKR | CAGUACCUGCA | | CCCCGU | NO: 223 |
| | | YKAAFTECCQAA | GCAGUGCCCUU | | GGUCUU | and Poly |
| | | DKAACLLPKLDE | UCGAGGACCAC | | UGAAUA | A tail of |
| | | LRDEGKASSAKQ | GUGAAGCUGGU | | AAGUCU | SEQ ID |
| | | RLKCASLQKFGE | GAACGAGGUGA | | GAGUGG | NO: 25 |
| | | RAFKAWAVARLS | CCGAGUUCGCCA | | GCGGC | |
| | | QRFPKAEFAEVS | AGACCUGCGUG | | | |
| | | KLVTDLTKVHTE | GCCGACGAGAG | | | |
| | | CCHGDLLECADD | CGCCGAGAACU | | | |
| | | RADLAKYICENQ | GCGACAAGAGC | | | |
| | | DSISSKLKECCEK | CUGCACACCCUG | | | |
| | | PLLEKSHCIAEVE | UUCGGCGACAA | | | |
| | | NDEMPADLPSLA | GCUGUGCACCG | | | |
| | | ADFVESKDVCKN | UGGCCACCCUGA | | | |
| | | YAEAKDVFLGMF | GAGAAACUUAC | | | |
| | | LYEYARRHPDYS | GGCGAGAUGGC | | | |
| | | VVLLLRLAKTYE | CGACUGCUGCGC | | | |
| | | TTLEKCCAAADP | CAAGCAGGAGC | | | |
| | | HECYAKVFDEFK | CAGAGCGGAAC | | | |
| | | PLVEEPQNLIKQN | GAGUGCUUCCU | | | |
| | | CELFEQLGEYKF | GCAACACAAGG | | | |
| | | QNALLVRYTKKV | ACGACAACCCUA | | | |
| | | PQVSTPTLVEVSR | ACCUGCCUAGAC | | | |
| | | NLGKVGSKCCKH | UGGUCCGGCCU | | | |
| | | PEAKRMPCAEDY | GAGGUGGACGU | | | |
| | | LSVVLNQLCVLH | GAUGUGCACGG | | | |
| | | EKTPVSDRVTKC | CCUUCCACGACA | | | |
| | | CTESLVNRRPCFS | ACGAGGAGACU | | | |
| | | ALEVDETYVPKE | UUCCUGAAGAA | | | |
| | | FNAETFTFHADIC | GUACCUGUACG | | | |
| | | TLSEKERQIKKQT | AGAUCGCCAGA | | | |
| | | ALVELVKHKPKA | AGACACCCUUAC | | | |
| | | TKEQLKAVMDDF | UUCUACGCCCCU | | | |
| | | AAFVEKCCKADD | GAGCUCCUGUU | | | |
| | | KETCFAEEGKKL | CUUCGCGAAGA | | | |
| | | VAASQAALGLGG | GAUACAAGGCC | | | |
| | | GSAPARSPSPSTQ | GCCUUCACCGAG | | | |
| | | PWEHVNAIQEAR | UGCUGCCAGGCC | | | |
| | | RLLNLSRDTAAE | GCCGACAAGGC | | | |
| | | MNETVEVISEMF | AGCUUGCCUGC | | | |
| | | DLQEPTCLQTRLE | UGCCUAAGCUG | | | |
| | | LYKQGLRGSLTK | GACGAGCUGAG | | | |
| | | LKGPLTMMASHY | AGACGAGGGCA | | | |
| | | KQHCPPTPETSCA | AGGCCUCCUCAG | | | |
| | | TQIITFESFKENLK | CUAAGCAGAGA | | | |
| | | DFLLVIPFDCWEP | CUGAAGUGCGC | | | |
| | | VQE | CAGCCUGCAGA | | | |
| | | | AGUUCGGUGAG | | | |
| | | | AGAGCAUUCAA | | | |
| | | | GGCUUGGGCCG | | | |
| | | | UCGCAAGACUG | | | |
| | | | UCACAGAGAUU | | | |
| | | | CCCUAAGGCAG | | | |
| | | | AAUUCGCGGAG | | | |
| | | | GUGAGCAAGCU | | | |
| | | | AGUGACCGACC | | | |
| | | | UGACCAAGGUG | | | |
| | | | CAUACAGAGUG | | | |
| | | | CUGCCACGGCGA | | | |
| | | | CCUGCUGGAGU | | | |
| | | | GCGCCGACGACA | | | |
| | | | GAGCCGACCUG | | | |
| | | | GCCAAGUACAU | | | |
| | | | CUGCGAGAACC | | | |
| | | | AGGACAGCAUC | | | |
| | | | AGCUCCAAGCU | | | |
| | | | GAAGGAGUGCU | | | |
| | | | GUGAGAAGCCU | | | |
| | | | CUGCUGGAGAA | | | |
| | | | GUCACACUGCA | | | |
| | | | UUGCCGAGGUC | | | |
| | | | GAGAACGACGA | | | |
| | | | GAUGCCUGCCG | | | |
| | | | AUCUUCCUAGCC | | | |
| | | | UUGCCGCCGAU | | | |
| | | | UUCGUGGAGAG | | | |
| | | | CAAGGACGUGU | | | |
| | | | GCAAGAACUAC | | | |
| | | | GCCGAGGCAAA | | | |
| | | | GGACGUGUUCC | | | |
| | | | UGGGCAUGUUC | | | |
| | | | CUUUACGAAUA | | | |
| | | | CGCUCGCCGGCA | | | |
| | | | UCCAGACUACA | | | |
| | | | GCGUGGUGCUG | | | |
| | | | CUGCUGAGAUU | | | |
| | | | GGCCAAGACUU | | | |
| | | | ACGAGACGACCC | | | |
| | | | UCGAGAAGUGU | | | |
| | | | UGCGCAGCAGC | | | |
| | | | UGAUCCUCACG | | | |
| | | | AGUGUUACGCC | | | |
| | | | AAGGUGUUCGA | | | |
| | | | CGAGUUCAAGC | | | |
| | | | CGCUUGUGGAG | | | |
| | | | GAGCCUCAGAA | | | |
| | | | CCUGAUCAAGC | | | |
| | | | AGAAUUGUGAG | | | |
| | | | CUGUUCGAGCA | | | |
| | | | GCUGGGUGAGU | | | |
| | | | ACAAGUUCCAG | | | |
| | | | AACGCCCUGCUG | | | |
| | | | GUGCGCUACACC | | | |
| | | | AAGAAGGUGCC | | | |
| | | | UCAAGUGUCUA | | | |
| | | | CCCCUACCCUGG | | | |
| | | | UUGAAGUUUCC | | | |
| | | | CGCAACCUGGGC | | | |
| | | | AAGGUGGGCAG | | | |
| | | | CAAGUGCUGCA | | | |
| | | | AGCAUCCUGAA | | | |
| | | | GCAAAGAGGAU | | | |
| | | | GCCUUGCGCCGA | | | |
| | | | GGACUACCUGU | | | |
| | | | CAGUGGUCCUU | | | |
| | | | AACCAGCUGUG | | | |
| | | | CGUGCUGCACG | | | |
| | | | AGAAGACCCCU | | | |
| | | | GUGAGCGACAG | | | |
| | | | AGUGACAAAGU | | | |
| | | | GUUGUACCGAG | | | |
| | | | AGCCUGGUCAA | | | |
| | | | CAGAAGACCUU | | | |
| | | | GCUUCAGCGCCC | | | |
| | | | UGGAAGUCGAC | | | |
| | | | GAGACAUACGU | | | |
| | | | GCCUAAGGAGU | | | |
| | | | UCAACGCCGAA | | | |
| | | | ACCUUCACCUUC | | | |
| | | | CACGCCGACAUC | | | |
| | | | UGCACACUGAG | | | |
| | | | CGAGAAGGAGA | | | |
| | | | GACAGAUCAAG | | | |
| | | | AAGCAGACCGCC | | | |
| | | | CUGGUCGAGUU | | | |
| | | | GGUGAAGCACA | | | |
| | | | AGCCUAAGGCC | | | |
| | | | ACCAAGGAGCA | | | |
| | | | ACUCAAGGCCG | | | |
| | | | UGAUGGACGAC | | | |
| | | | UUCGCGGCCUUC | | | |
| | | | GUUGAGAAGUG | | | |
| | | | CUGUAAGGCUG | | | |
| | | | ACGACAAGGAG | | | |
| | | | ACGUGCUUCGC | | | |
| | | | UGAGGAGGGUA | | | |
| | | | AGAAGCUUGUC | | | |
| | | | GCCGCCUCUCAG | | | |
| | | | GCCGCUUUGGG | | | |
| | | | ACUCGGCGGCG | | | |
| | | | GCAGUGCGCCU | | | |
| | | | GCCAGAAGCCCU | | | |
| | | | UCCCCAUCUACC | | | |
| | | | CAGCCUUGGGA | | | |
| | | | GCACGUGAACG | | | |
| | | | CCAUCCAGGAG | | | |
| | | | GCCAGACGUCU | | | |
| | | | GCUGAACCUGU | | | |
| | | | CACGGGAUACC | | | |
| | | | GCAGCUGAGAU | | | |
| | | | GAACGAAACUG | | | |
| | | | UUGAGGUCAUC | | | |
| | | | AGCGAGAUGUU | | | |
| | | | CGACCUACAGG | | | |
| | | | AACCUACCUGCU | | | |
| | | | UGCAGACCAGA | | | |
| | | | CUGGAGCUGUA | | | |
| | | | CAAGCAGGGAU | | | |
| | | | UAAGAGGCUCC | | | |
| | | | CUGACGAAGCU | | | |
| | | | UAAGGGCCCUC | | | |
| | | | UGACCAUGAUG | | | |
| | | | GCCAGCCACUAU | | | |
| | | | AAGCAGCACUG | | | |
| | | | CCCUCCUACCCC | | | |
| | | | UGAAACGUCGU | | | |
| | | | GUGCUACCCAG | | | |
| | | | AUCAUCACCUUC | | | |
| | | | GAGAGCUUCAA | | | |
| | | | GGAGAAUCUGA | | | |
| | | | AGGACUUCCUG | | | |
| | | | CUCGUUAUUCC | | | |
| | | | GUUCGAUUGUU | | | |
| | | | GGGAGCCUGUG | | | |
| | | | CAGGAG | | | |

Note:
“G5” indicates that all uracils (U) in the mRNA are replaced by N1-methylpseudouracils.

### Lipid Content of LNPs

As set forth above, with respect to lipids, LNPs disclosed herein comprise an (i) ionizable lipid; (ii) sterol or other structural lipid; (iii) a non-cationic helper lipid or phospholipid; and 0 a (iv) PEG lipid. These categories of lipids are set forth in more detail below.

### Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs.

In some aspects, the ionizable lipids of the present disclosure may be one or more of compounds of Formula (I):

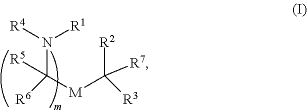

or their N-oxides, or salts or isomers thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, and each n is independently selected from 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected
from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of Cis alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R⁴ is (CH₂)ₙQ, (CH₂)ₙCHQR, —CHQR, or CQ(R)₂, then (i) Q is not N(R)₂ when n is 1, 2, 3, 4 or 5, or (ii) Q is not 5, 6, or 7-membered heterocycloalkyl when n is 1 or 2.

In one embodiment, the compounds of Formula (I) are of Formula (IIa),

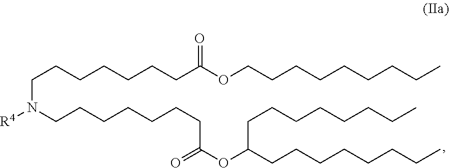

or their N-oxides, or salts or isomers thereof, wherein R⁴ is as described herein.
In another embodiment, the compounds of Formula (I) are of Formula (IIb),

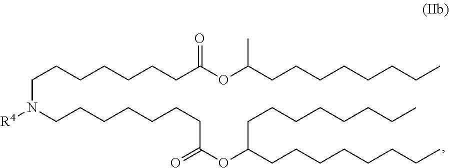

or their N-oxides, or salts or isomers thereof, wherein R⁴ is as described herein.

The structure of ionizable lipids of the disclosure include the prefix I to distinguish them from other lipids of the invention.

In an embodiment, the LNP comprises an ionizable lipid comprising compound I-18.

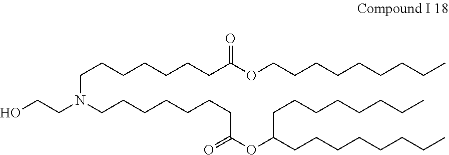 [SMILES: CCCCCCCCCOC(=O)CCCCCCCN(CCO)CCCCCCCC(=O)OC(CCCCCCCC)CCCCCCCC]

In an embodiment, the LNP comprises an ionizable lipid comprising compound I-25.

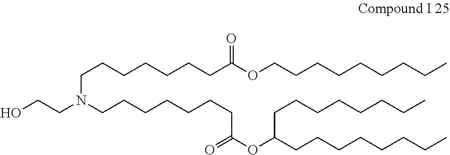 [SMILES: CCCCCCCCCOC(=O)CCCCCCCN(CCO)CCCCCCCC(=O)OC(CCCCCCCC)CCCCCCCC]

### Cholesterol/Structural Lipids

The LNP described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol.

Non-Cationic Helper Lipids/Phospholipids In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

### Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

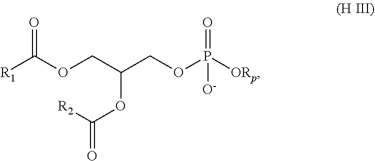

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.

Phospholipids useful in the compositions and methods described herein may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine(22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE(18:2/18:2), 1,2-dilinolenoyl-sn-glycero-3-phosphoethanolamine (PE 18:3(9Z, 12Z, 15Z), 1,2-diarachidonoyl-sn-glycero-3-phosphoethanolamine (DAPE 18:3 (9Z, 12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin. Each possibility represents a separate embodiment of the invention.

In some embodiments, an LNP includes DSPC. In certain embodiments, an LNP includes DOPE. In some embodiments, an LNP includes DMPE. In some embodiments, an LNP includes both DSPC and DOPE.

In one embodiment, a non-cationic helper lipid for use in a target cell target cell delivery LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

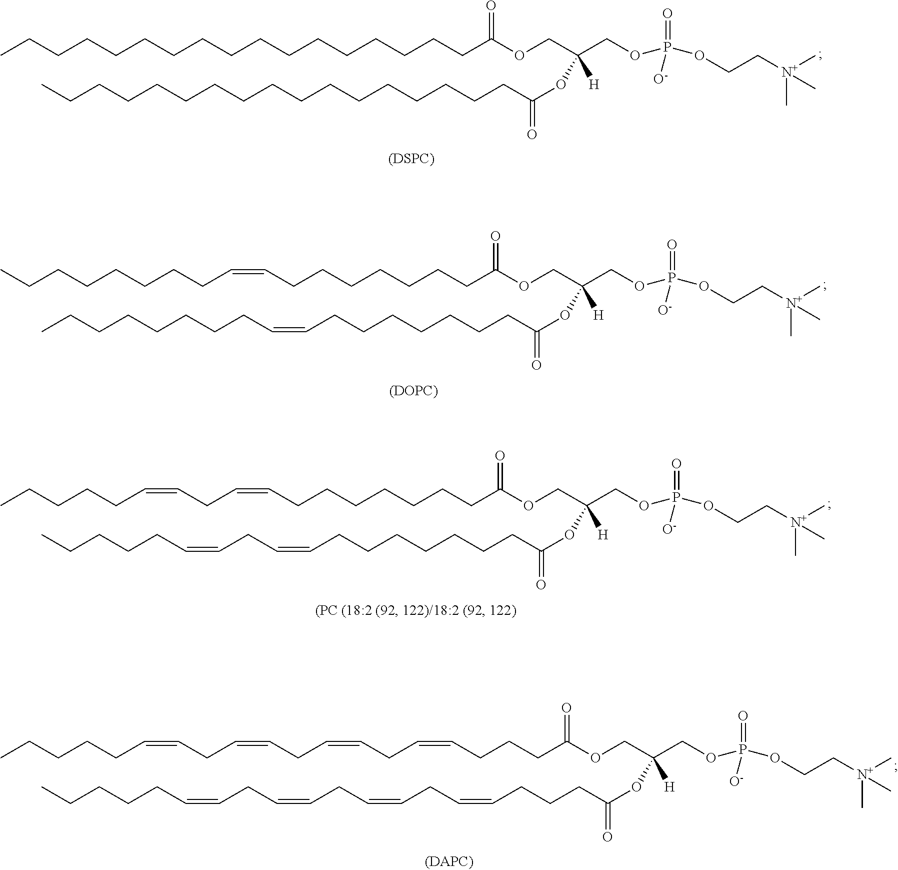

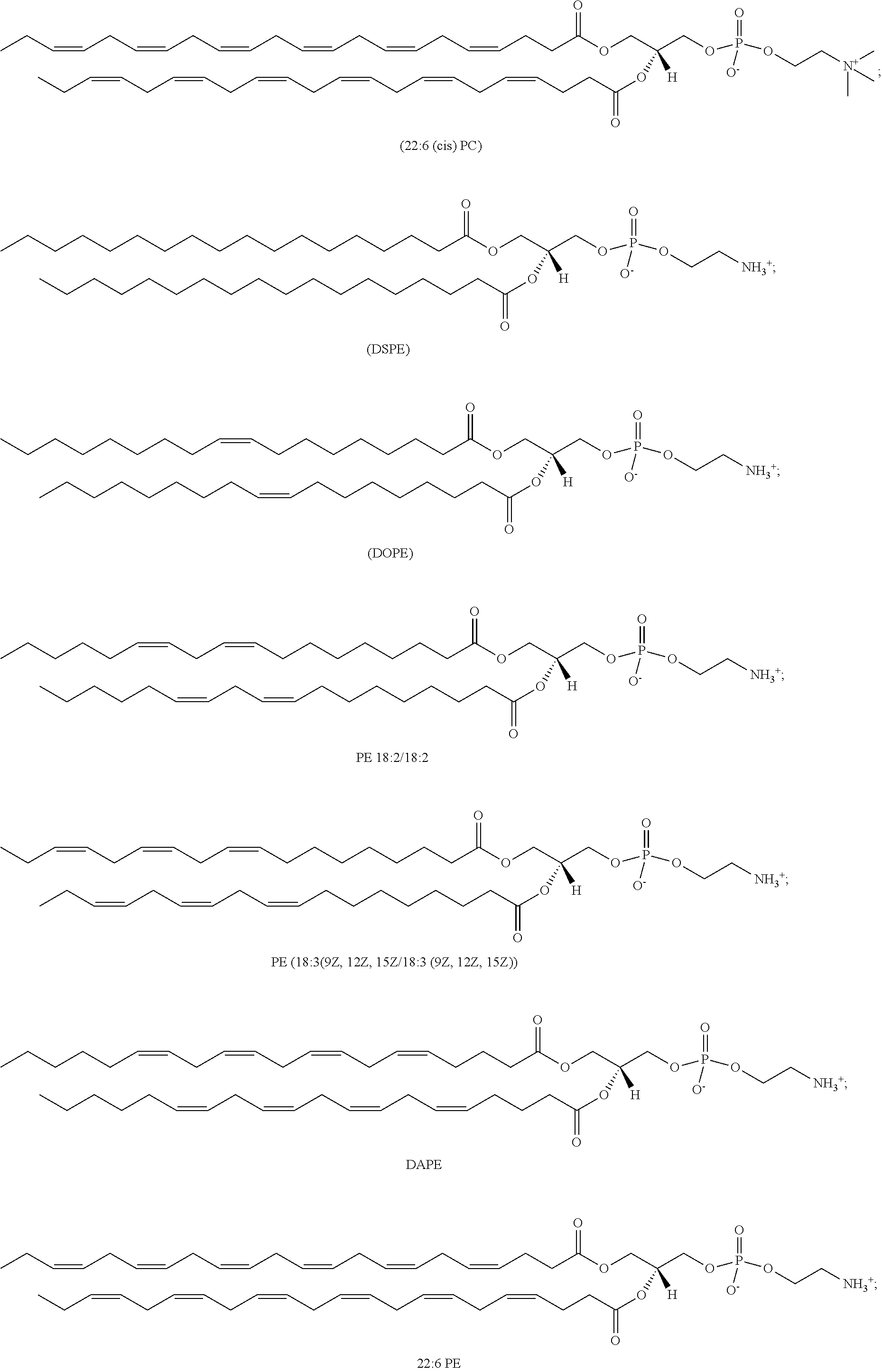

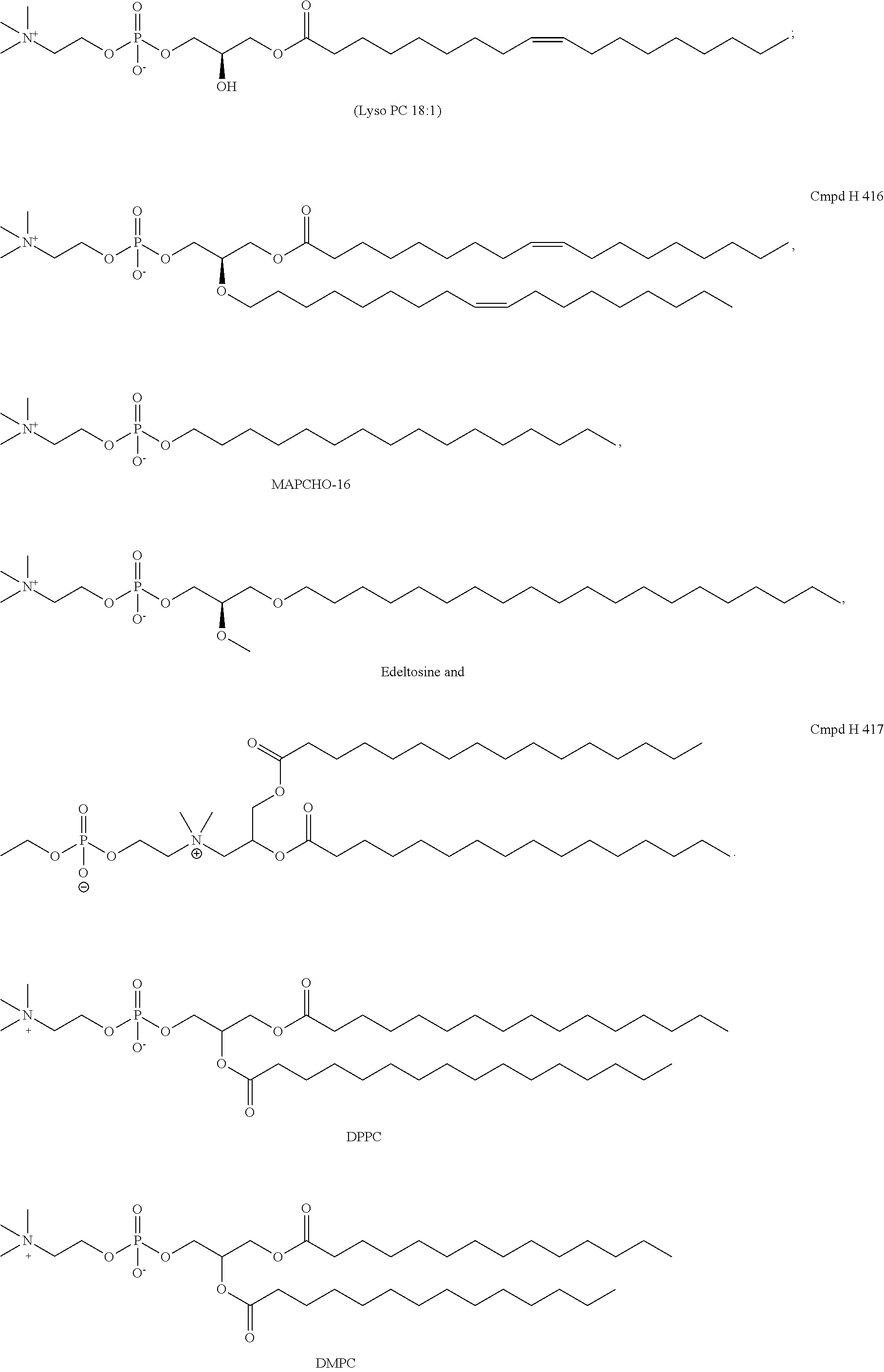

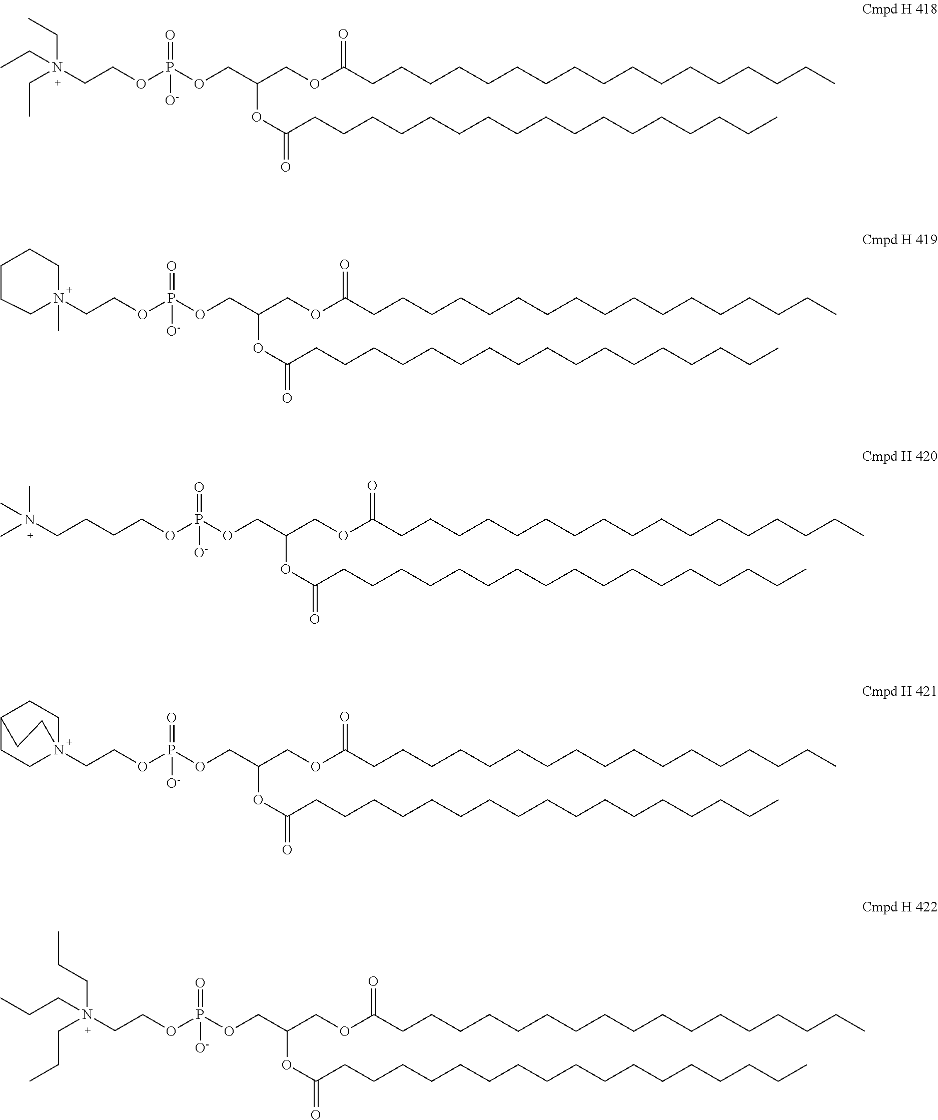

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

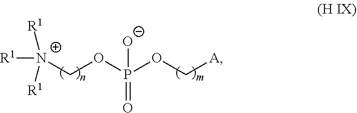

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula: or

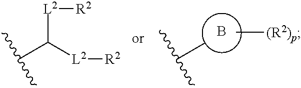

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}CO—, —C(O)), —CO—(O)—, —C(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N}) —C(O)O—, —OC(O)—, —OC(O)O—, —OC(O)N(R^{N})—, —NRNC(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S) —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

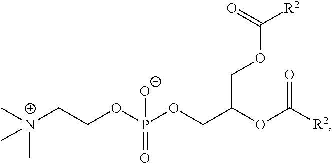

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

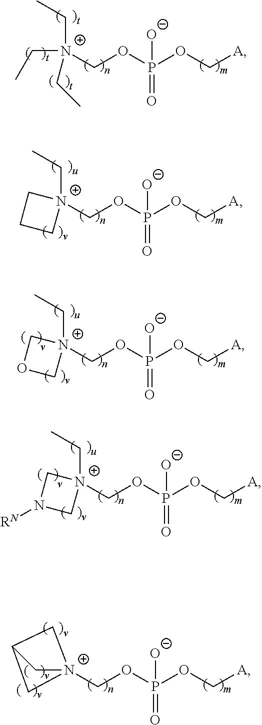

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (H IX) is of one of the following formulae:

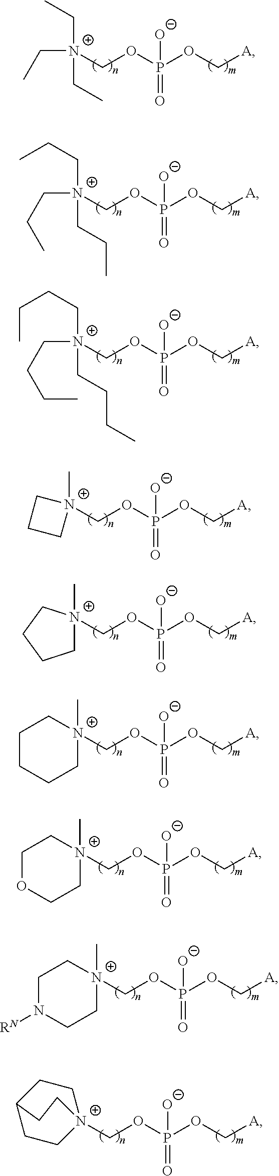

or a salt thereof.

In certain embodiments, a compound of Formula (H IX) is one of the following:

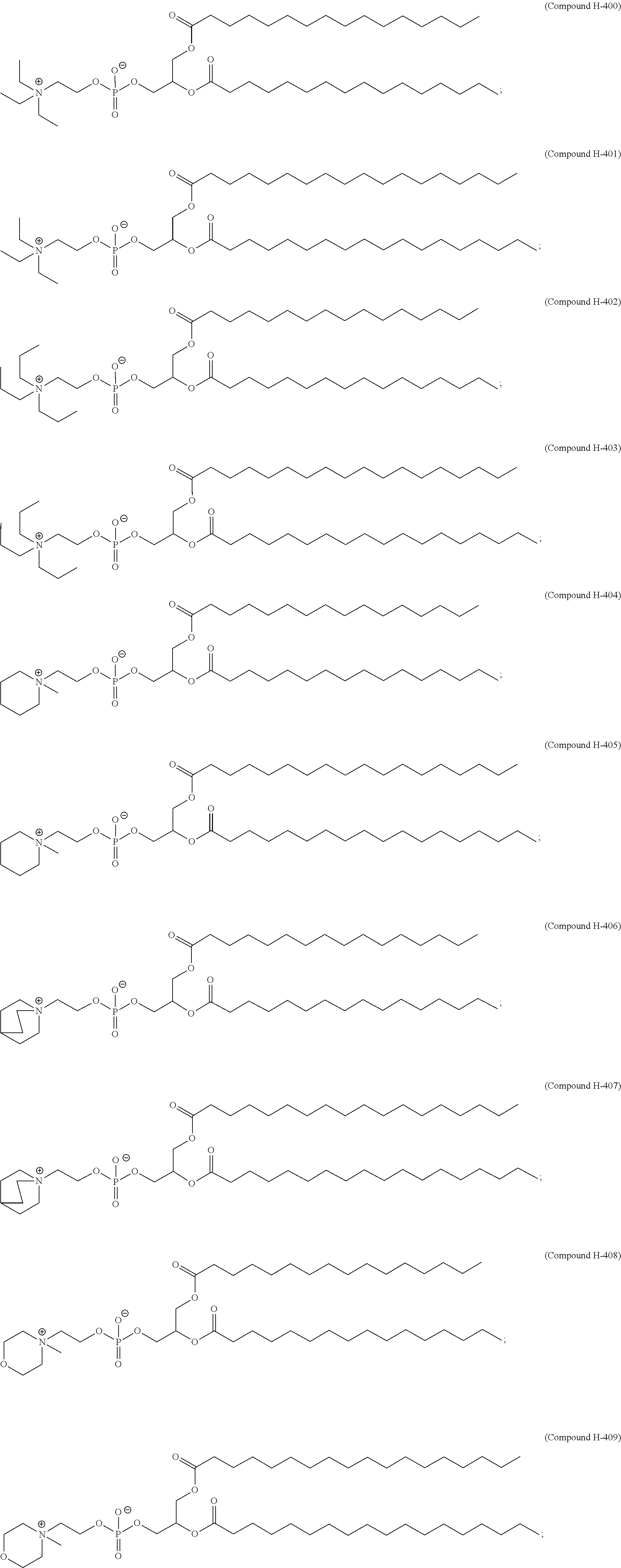

or a salt thereof.

In one embodiment, a target cell target cell delivery LNP comprises Compound H-409 as a non-cationic helper lipid.

### (ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)C—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H IX) is of Formula (H IX-c):

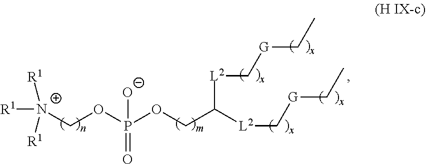

or a salt thereof, wherein:
each x is independently an integer between 0-30, inclusive; and
   each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H IX-c) is of Formula (H IX-c-1):

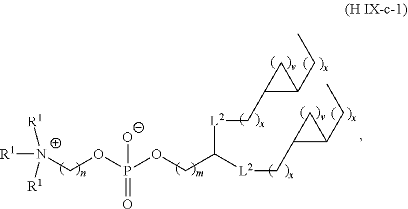

or salt thereof, wherein:
each instance of v is independently 1, 2, or 3.

In certain embodiments, the compound of Formula (H IX-c) is of Formula (H IX-c-2):

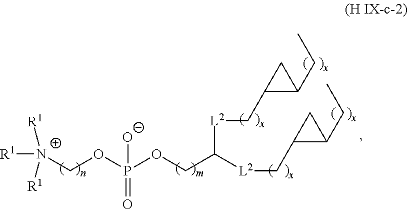

or a salt thereof.

In certain embodiments, the compound of Formula (IX-c) is of the following formula:

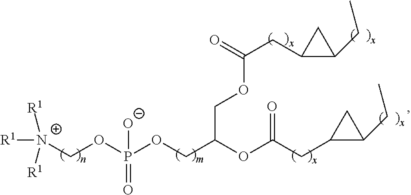

or a salt thereof.

In certain embodiments, the compound of Formula (H IX-c) is the following:

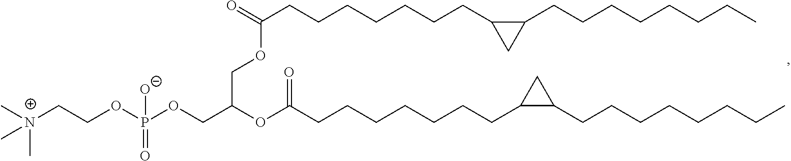 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H IX-c) is of Formula (H IX-c-3):

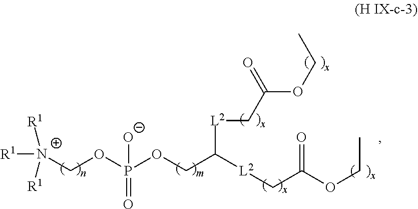

or a salt thereof.

In certain embodiments, the compound of Formula (H IX-c) is of the following formulae:

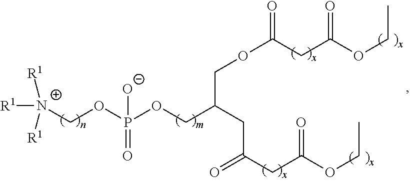

or a salt thereof.

In certain embodiments, the compound of Formula (H IX-c) is the following:

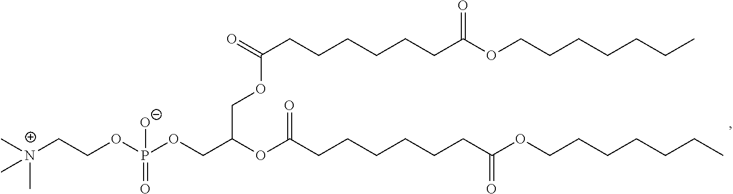 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

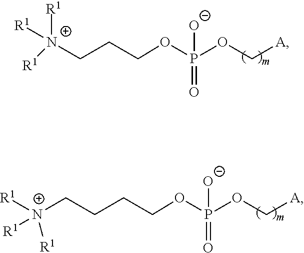

or a salt thereof.

In certain embodiments, a compound of Formula (H IX) is one of the following:

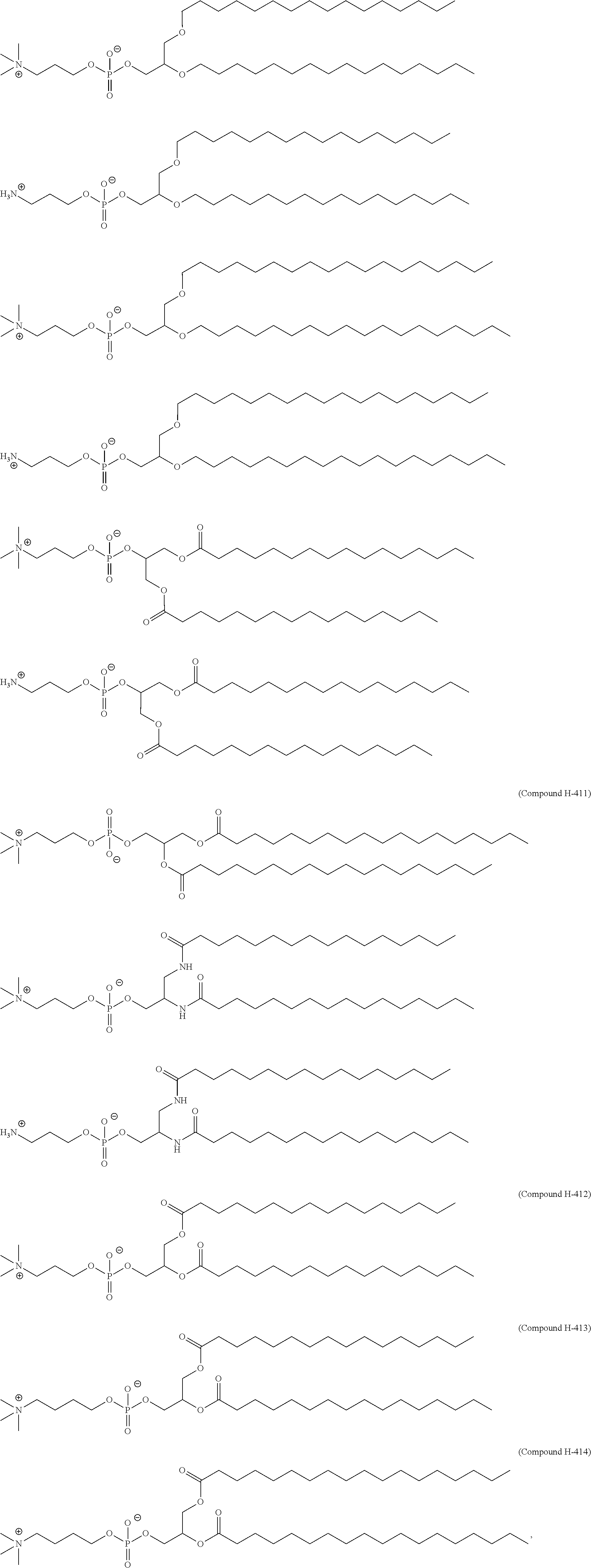

or salts thereof.

In certain embodiments, an alternative lipid is used in place of a phospholipid of the invention. Non-limiting examples of such alternative lipids include the following:

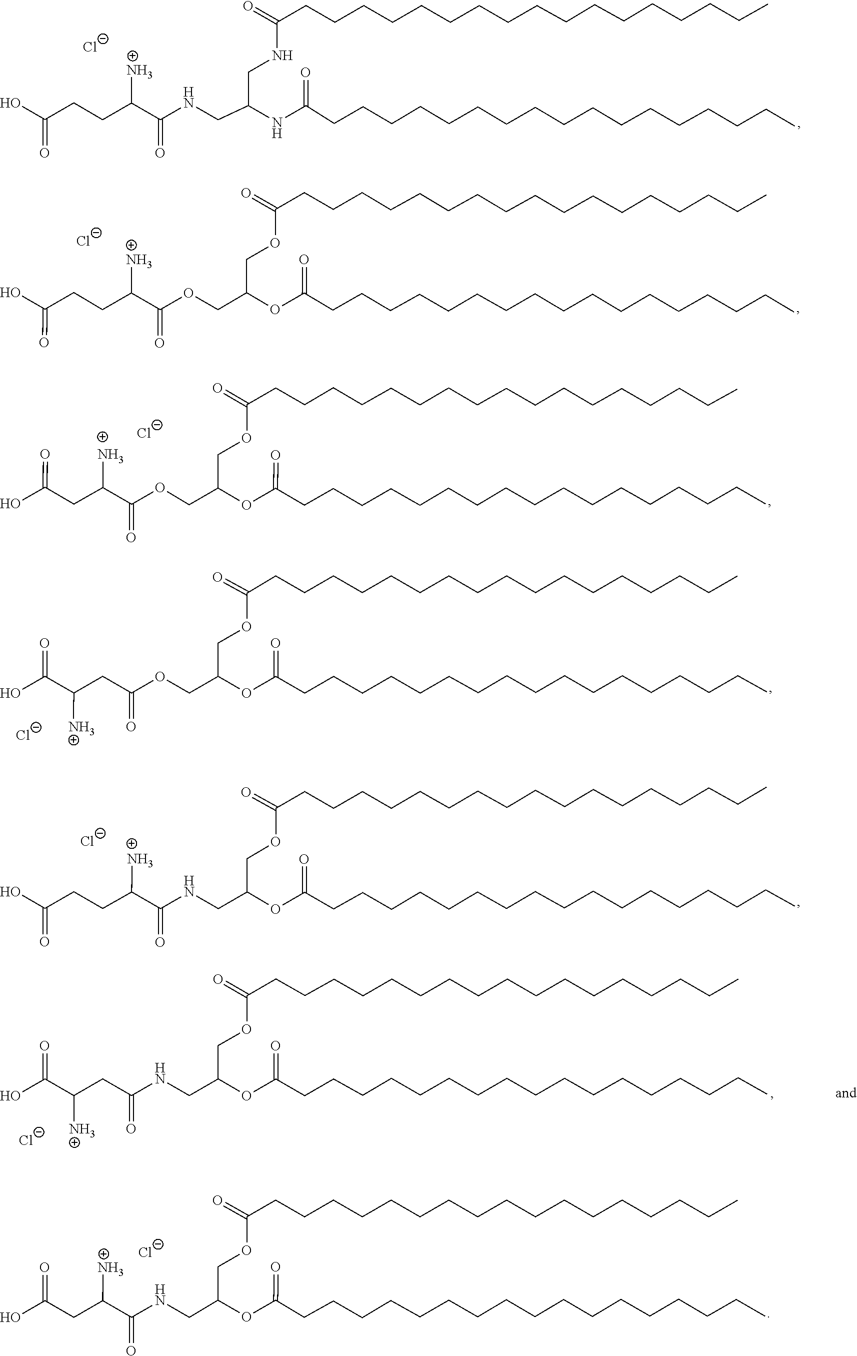

Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N}) —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S) —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In certain embodiments, the compound of Formula (H I-a) is of Formula (H I-c):

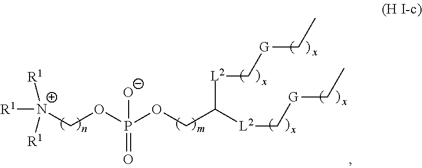

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H I-c) is of Formula (H I-c-1):

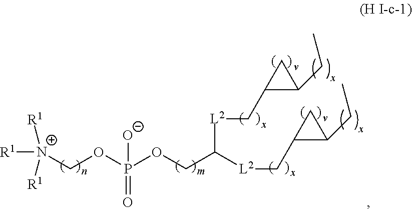

or salt thereof, wherein:
each instance of v is independently 1, 2, or 3.

In certain embodiments, the compound of Formula (H I-c) is of Formula (H I-c-2):

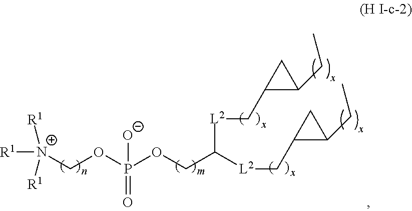

or a salt thereof.

In certain embodiments, the compound of Formula (I-c) is of the following formula:

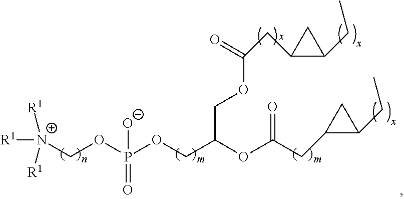

or a salt thereof.

In certain embodiments, the compound of Formula (H I-c) is the following:

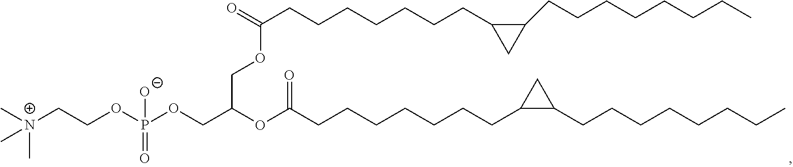 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

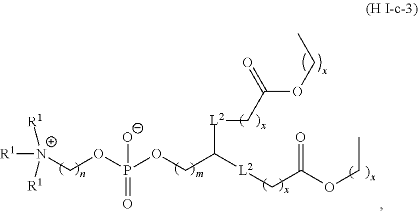

or a salt thereof.

In certain embodiments, the compound of Formula (H I-c) is of the following formulae:

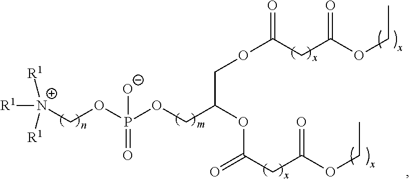

or a salt thereof.

In certain embodiments, the compound of Formula (H I-c) is the following:

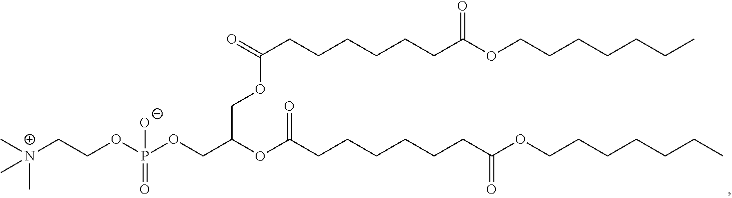 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

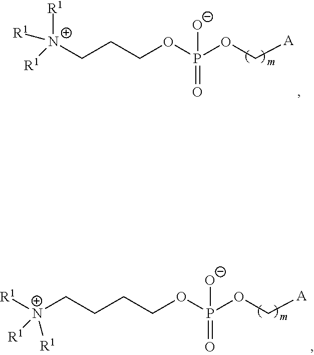

or a salt thereof.

In certain embodiments, a compound of Formula (H I) is one of the following:

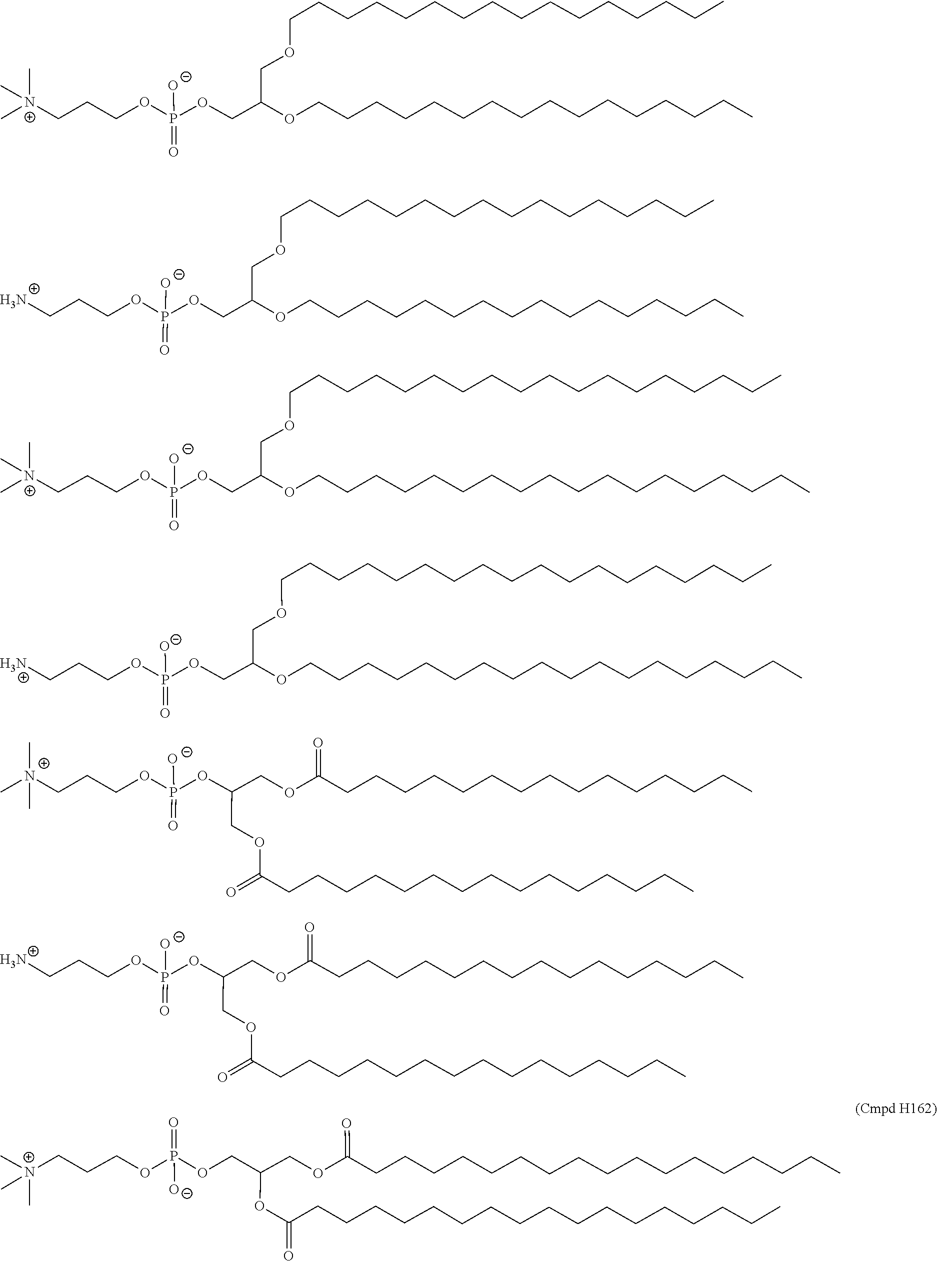

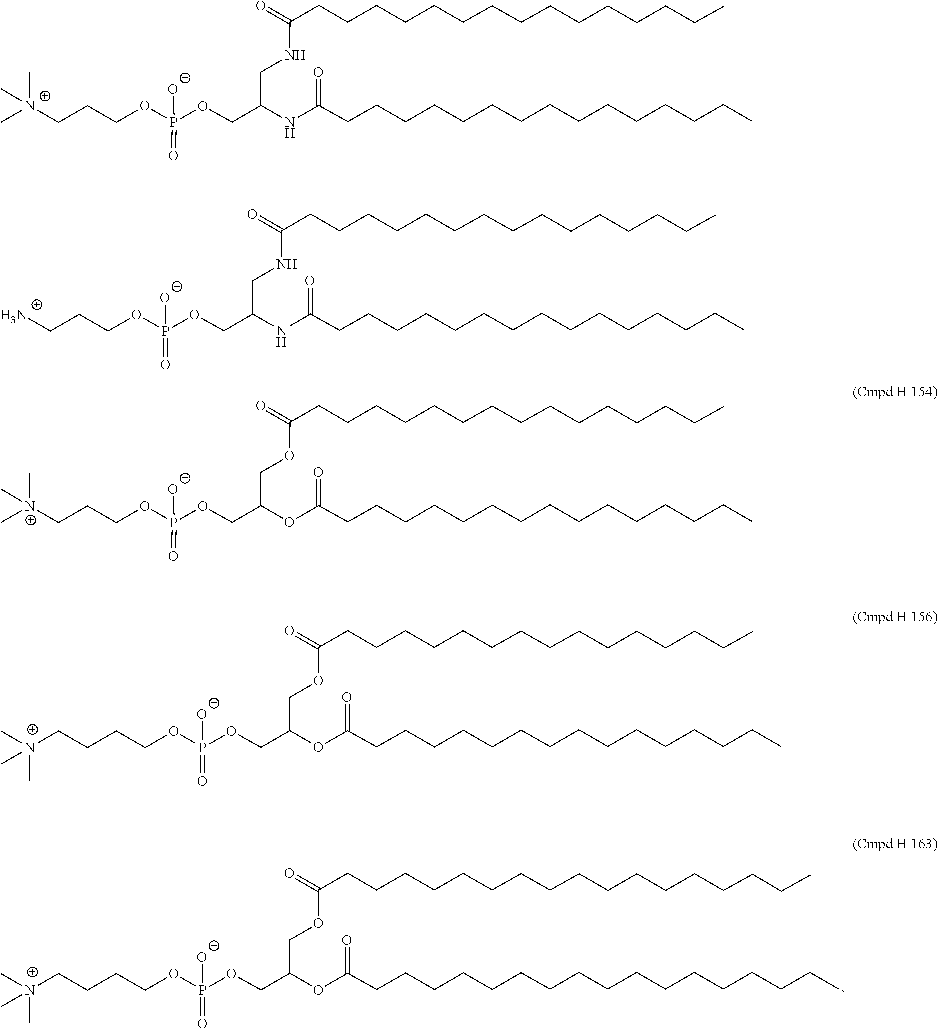

or salts thereof.

Numerous LNP formulations having phospholipids other than DSPC were prepared and tested for activity, as demonstrated in the examples below.

Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(i) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

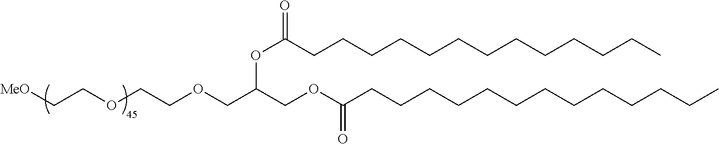

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (PI):

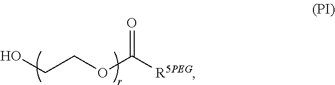

or a salt or isomer thereof, wherein:
r is an integer between 1 and 100;
R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N}) —NR^{N}C(═NR^{N})N(R^{N})—, —C(S)—, —C(S)N(R^{N})—, —NR^{N}C(S)—, —NR^{N}C(S)N(R^{N}) —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and each instance of R^{N} is independently hydrogen, C1-6 alkyl, or a nitrogen protecting group.

For example, R⁵PEG is C17 alkyl. For example, the PEG lipid is a compound of Formula (PI-a):

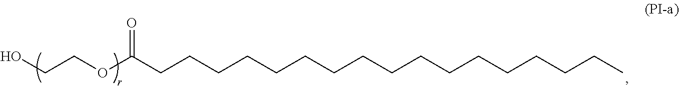

or a salt or isomer thereof, wherein r is an integer between 1 and 100.

For example, the PEG lipid is a compound of the following formula:

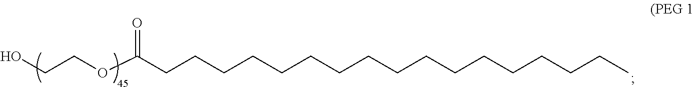

also referred to as Compound 428 below), or a salt or isomer thereof.

The PEG lipid may be a compound of Formula (PII):

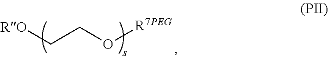

or a salt or isomer thereof, wherein:
s is an integer between 1 and 100;
R″ is a hydrogen, C1-10 alkyl, or an oxygen protecting group;
   R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N}) —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and each instance of R^{N} is independently hydrogen, C₁₋₆ alkyl, or a nitrogen protecting group.

In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

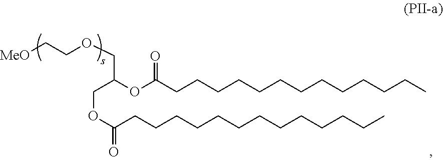

or a salt or isomer thereof, wherein s is an integer between 1 and 100.

For example, the PEG lipid is a compound of the following formula:

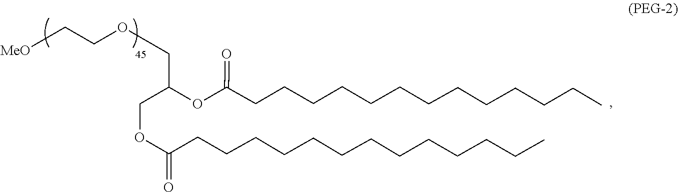

or a salt or isomer thereof.

In certain embodiments, a PEG lipid useful in the present invention is a compound of Formula (PIII). Provided herein are compounds of Formula (PIII):

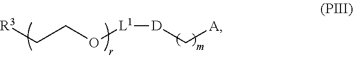

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, 0, N(R^{N}), S, C(O), C(O)N(R^{N}), NR^{N}C(O) C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

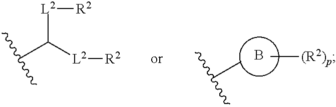

each instance of L² is independently a bond or optionally substituted C₁₋₆ alkylene, wherein one methylene unit of the optionally substituted C₁₋₆ alkylene is optionally replaced with 0, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

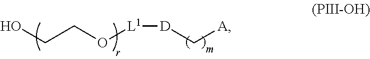

or a salt thereof.

In certain embodiments, D is a moiety obtained by click chemistry (e.g., triazole). In certain embodiments, the compound of Formula (PIII) is of Formula (PIII-a-1) or (PIII-a-2):

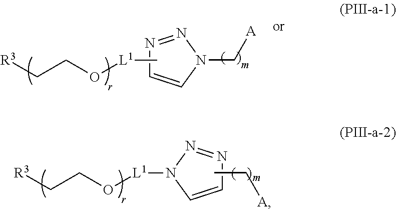

or a salt thereof.

In certain embodiments, the compound of Formula (PIII) is of one of the following formulae:

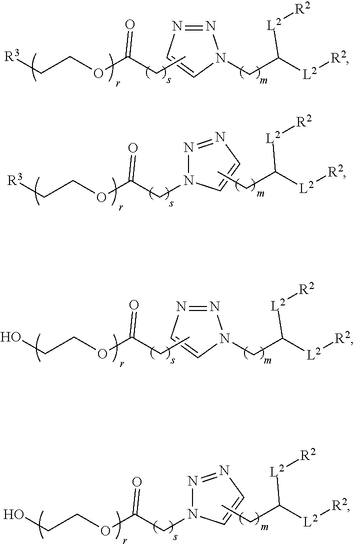

or a salt thereof, wherein
s is 0, 1, 2, 3, 4, 5, 6, 7, 8, 9, or 10.

In certain embodiments, the compound of Formula (PIII) is of one of the following formulae:

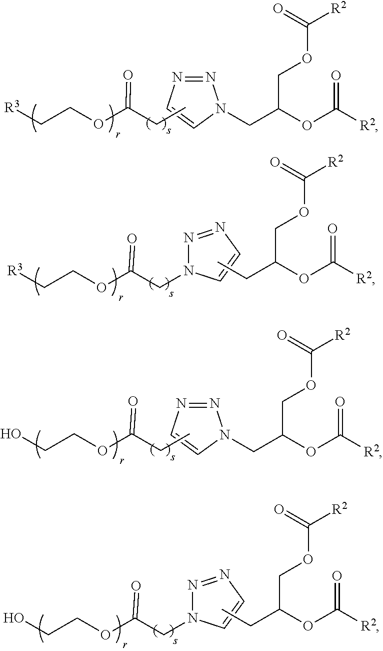

or a salt thereof.

In certain embodiments, a compound of Formula (PIII) is of one of the following formulae:

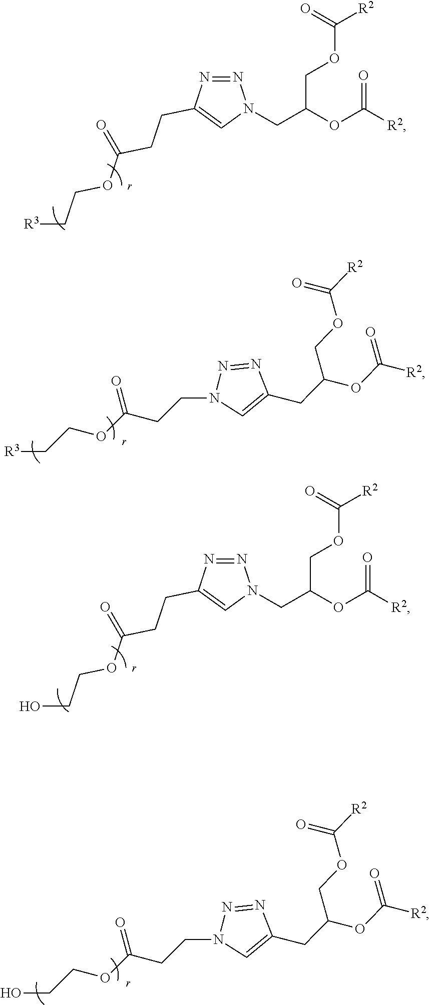

or a salt thereof.

In certain embodiments, a compound of Formula (PIII) is of one of the following formulae:

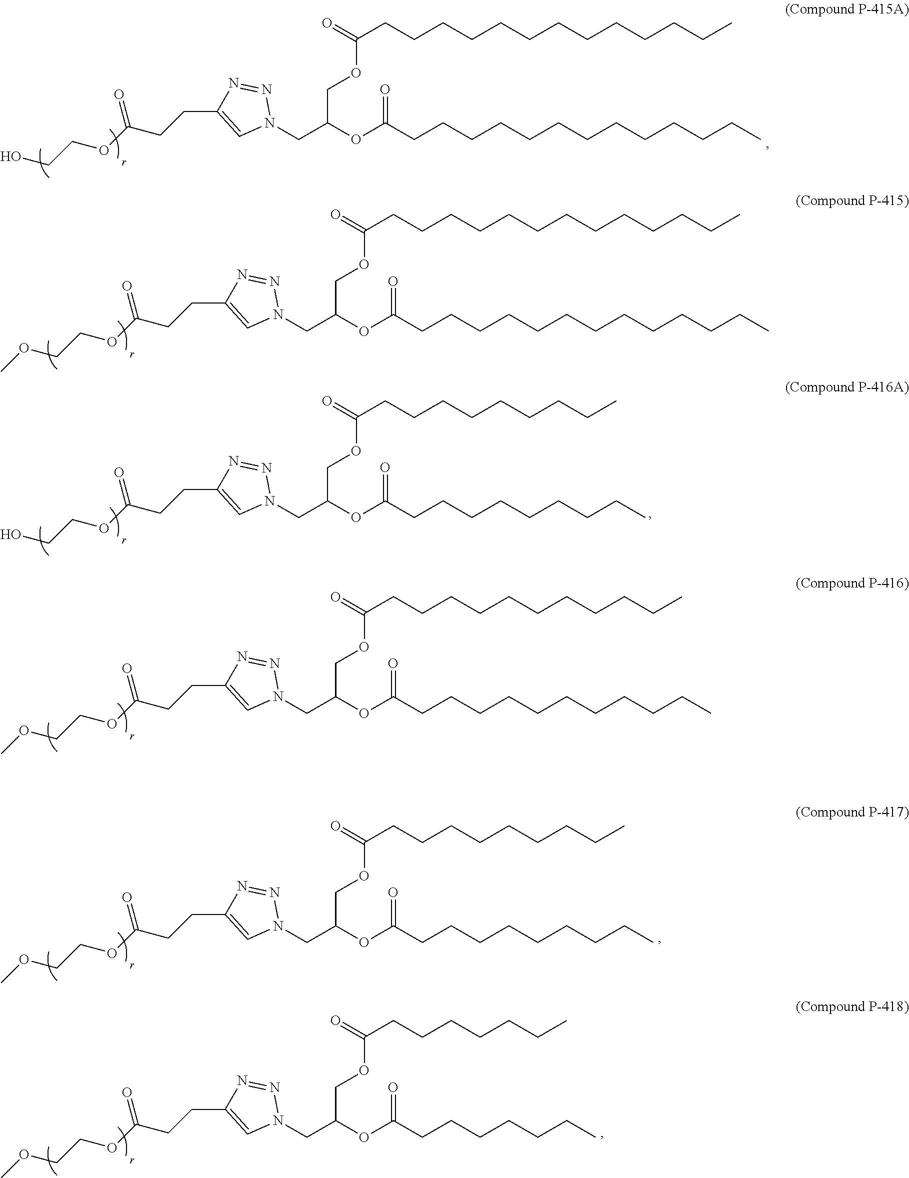

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

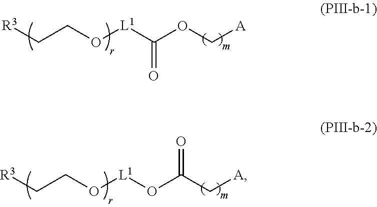

or a salt thereof.

In certain embodiments, a compound of Formula (PIII) is of Formula (PIII-b-1-OH) or (PIII-b-2-OH):

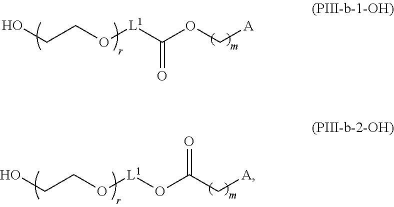

or a salt thereof.

In certain embodiments, the compound of Formula (PIII) is of one of the following formulae:

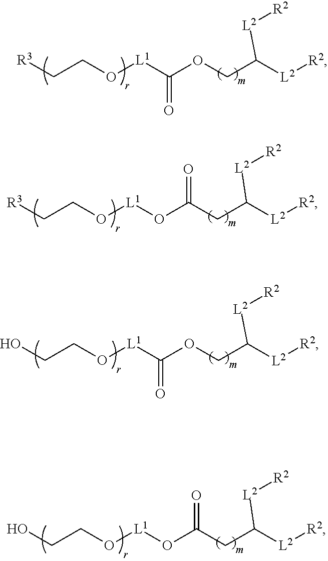

or a salt thereof.

In certain embodiments, a compound of Formula (PIII) is of one of the following formulae:

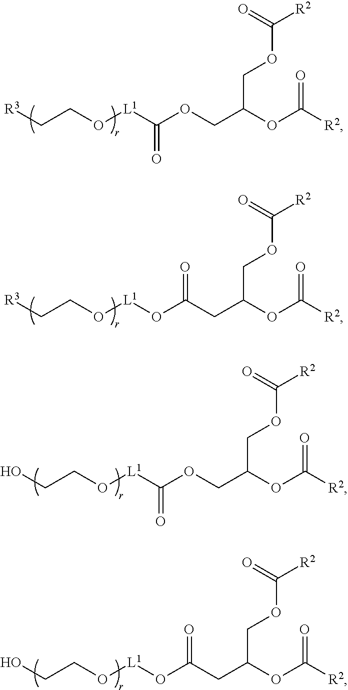

or a salt thereof.

In certain embodiments, a compound of Formula (PIII) is of one of the following formulae:

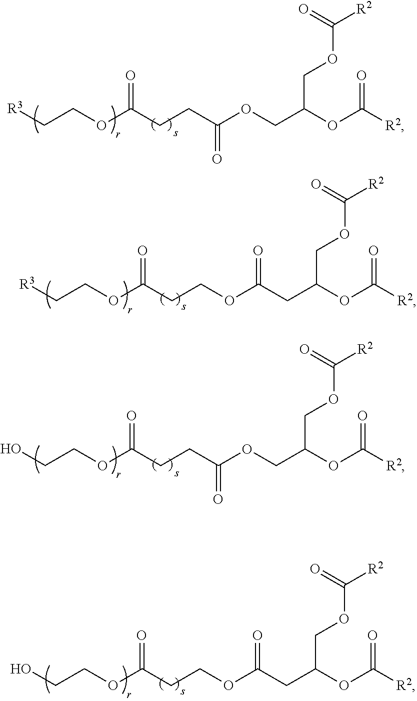

or a salt thereof.

In certain embodiments, a compound of Formula (PIII) is of one of the following formulae:

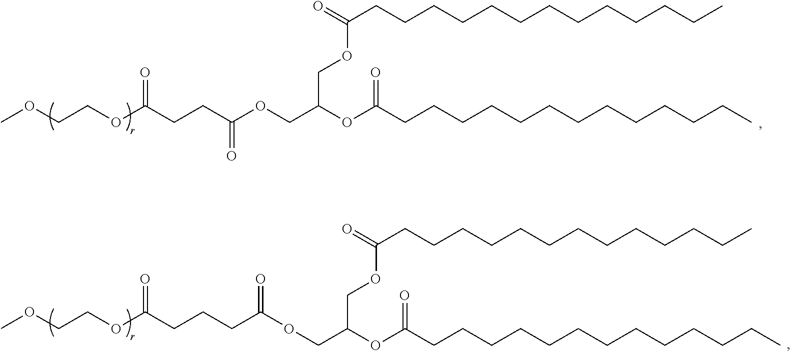

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

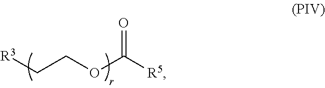

or a salts thereof, wherein:
R³ is-OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S) —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and each instance of R^{N} is independently hydrogen, optionally substituted alkyl, or a nitrogen protecting group.

In certain embodiments, the compound of Formula (PIV is of Formula (PIV-OH):

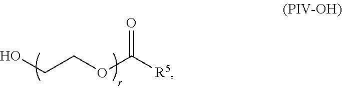

or a salt thereof. In some embodiments, r is 40-50. In some embodiments, r is 45.

In certain embodiments, a compound of Formula (PIV) is of one of the following

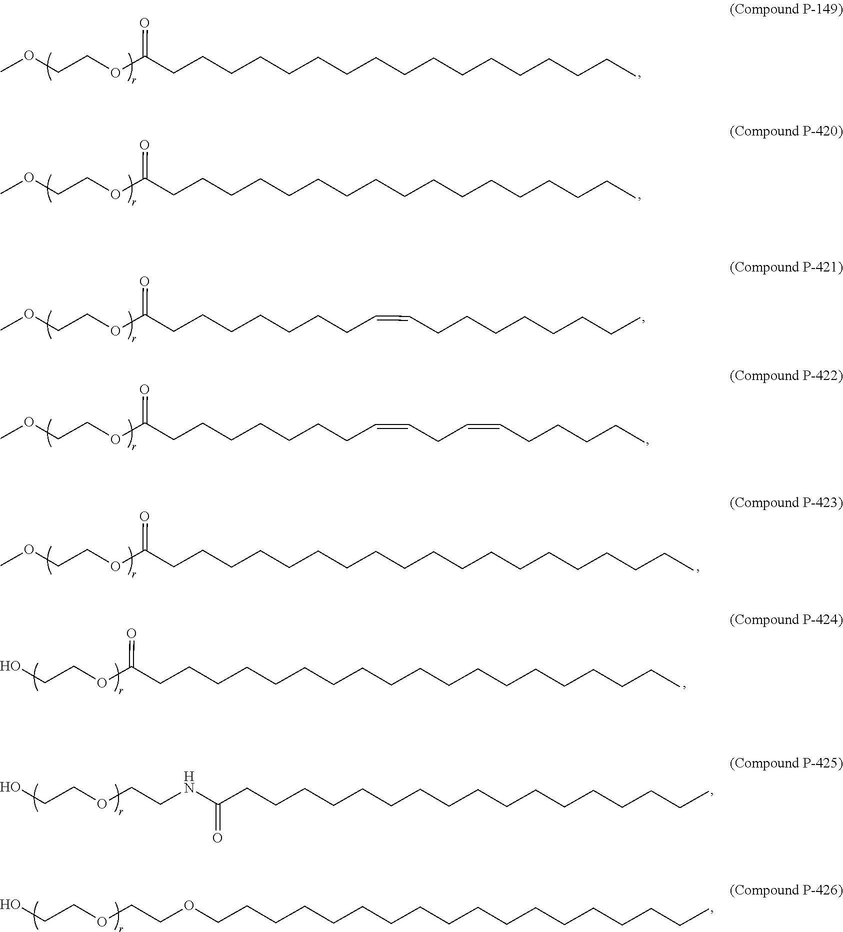

or a salt thereof. In some embodiments, r is 40-50. In some embodiments, r is 45.

In yet other embodiments the compound of Formula (PIV) is:

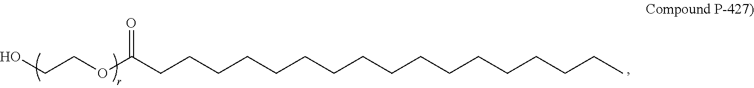

or a salt thereof.

In one embodiment, the compound of Formula (PIV) is

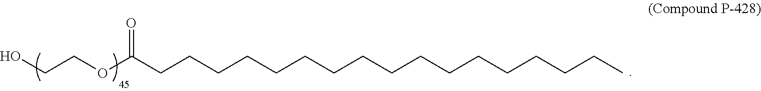

A skilled artisan understanding the polydispersity of polymeric compositions would appreciate that an n value of 45 (e.g., in a structural formula, such as P-428) can represent a distribution of values between 40-50 in an actual PEG-containing composition.

In one aspect, provided herein are lipid nanoparticles (LNPs) for use in the treatment of Parkinson's disease comprising PEG lipids of Formula (PV):

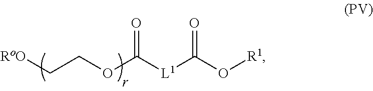

or pharmaceutically acceptable salts thereof, wherein:
L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;
R¹ is optionally substituted C₅₋₃₀ alkyl, optionally substituted C₅₋₃₀ alkenyl, or optionally substituted C₅₋₃₀ alkynyl;
R^{O} is hydrogen, optionally substituted alkyl, optionally substituted acyl, or an oxygen protecting group; and
r is an integer from 2 to 100, inclusive.

In certain embodiments, the PEG lipid of Formula (PV) is of the following formula:

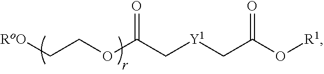

or a pharmaceutically acceptable salt thereof, wherein:
Y¹ is a bond, —CR₂—, —O—, —NR^{N}—, or —S—;
each instance of R is independently hydrogen, halogen, or optionally substituted alkyl; and
R^{N} is hydrogen, optionally substituted alkyl, optionally substituted acyl, or a nitrogen protecting group.

In certain embodiments, the PEG lipid of Formula (PV) is of one of the following formulae:

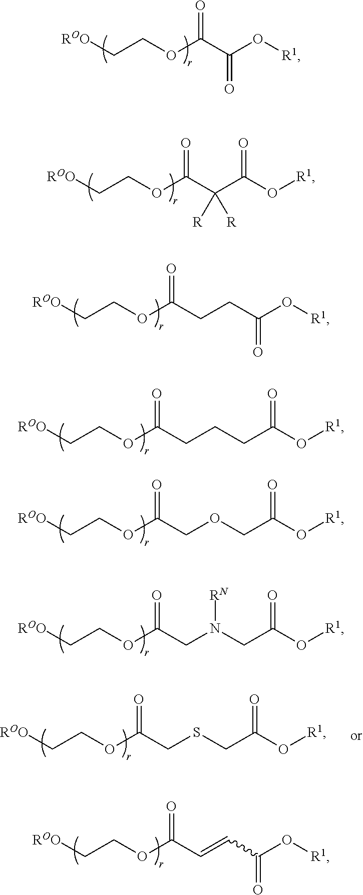

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

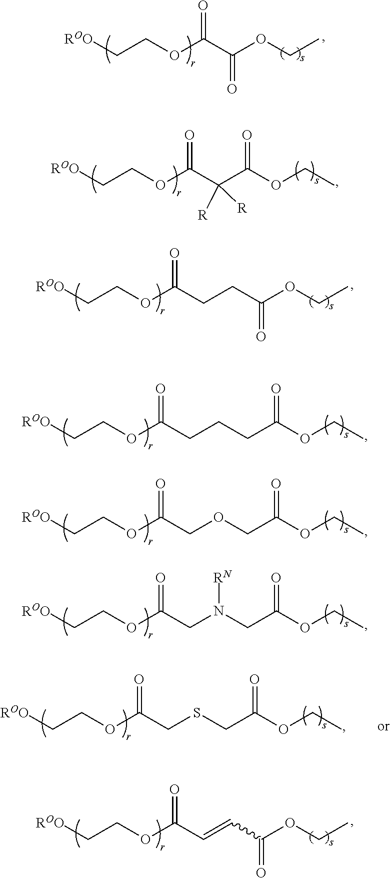

or a pharmaceutically acceptable salt thereof, wherein:
s is an integer from 5-25, inclusive.

In certain embodiments, the PEG lipid of Formula (PV) is of one of the following formulae:

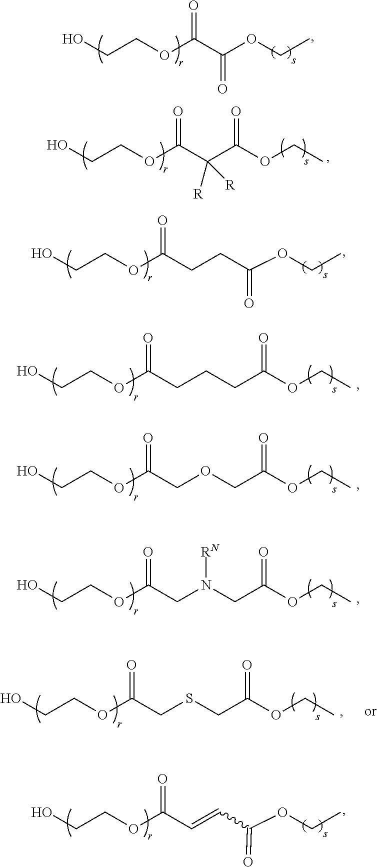

or a pharmaceutically acceptable salt thereof.

In certain embodiments, the PEG lipid of Formula (PV) is selected from the group consisting of

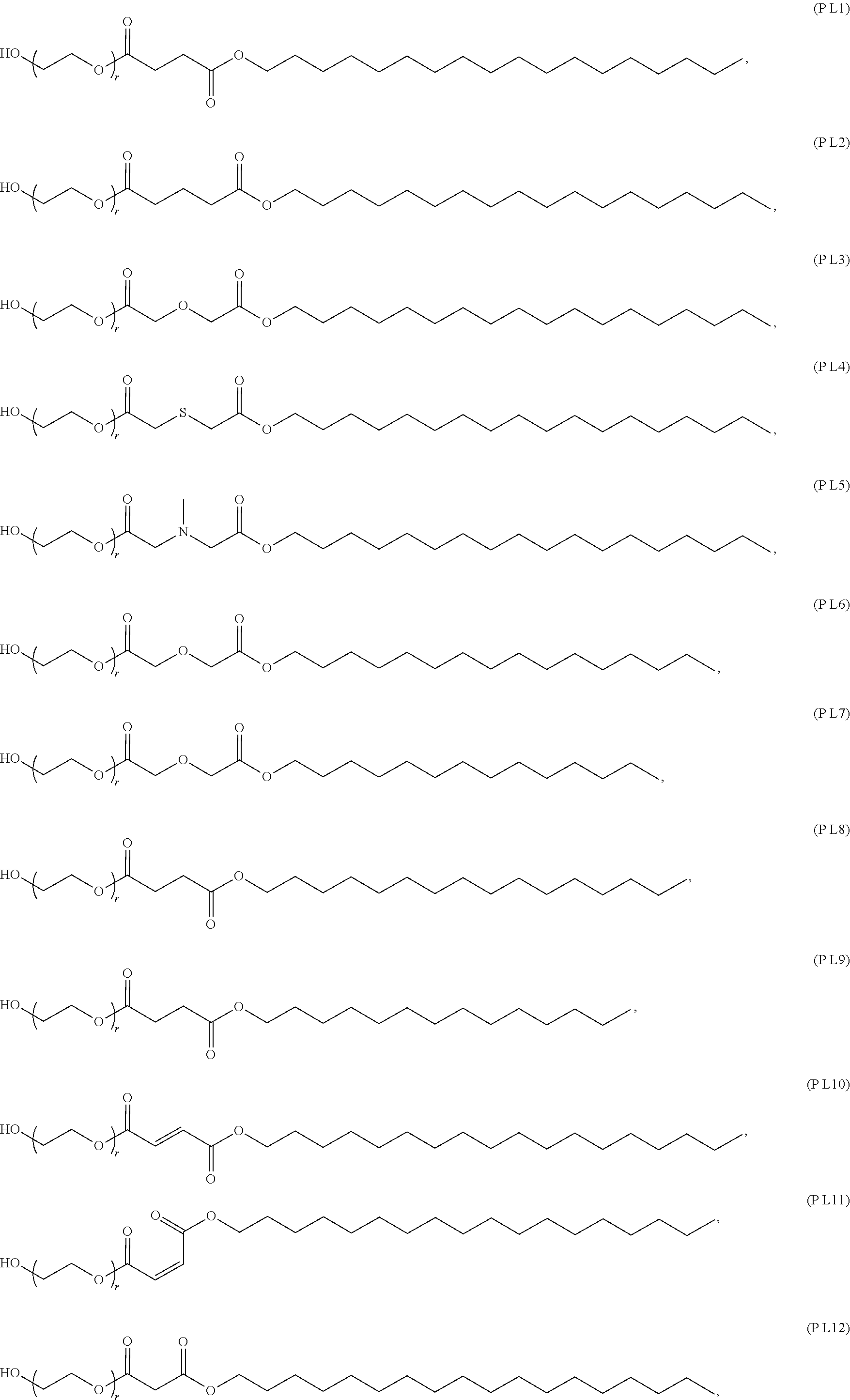

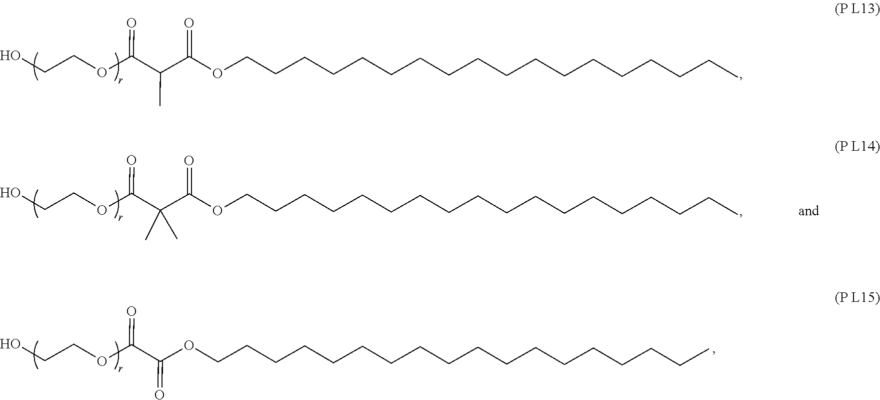

and pharmaceutically acceptable salts thereof.

In another aspect, provided herein are lipid nanoparticles (LNPs) for the treatment of Parkinson's disease comprising PEG lipids of Formula (PVI):

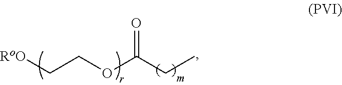

or pharmaceutically acceptable salts thereof; wherein:
R^{O} is hydrogen, optionally substituted alkyl, optionally substituted acyl, or an oxygen protecting group;
r is an integer from 2 to 100, inclusive; and
m is an integer from 5-15, inclusive, or an integer from 19-30, inclusive.

In certain embodiments, the PEG lipid of Formula (PVI) is of one of the following formulae:

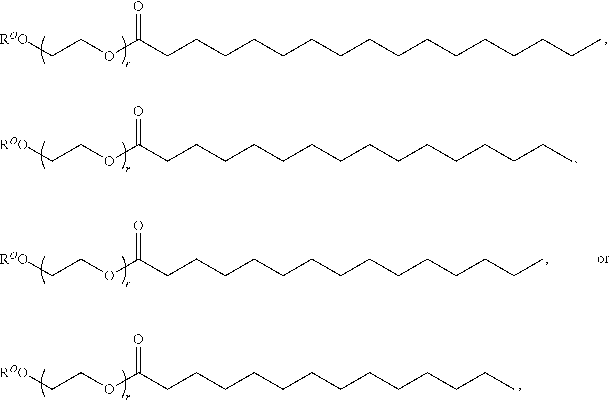

or a pharmaceutically acceptable salt thereof.

In certain embodiments, the PEG lipid of Formula (PVI) is of one of the following formulae:

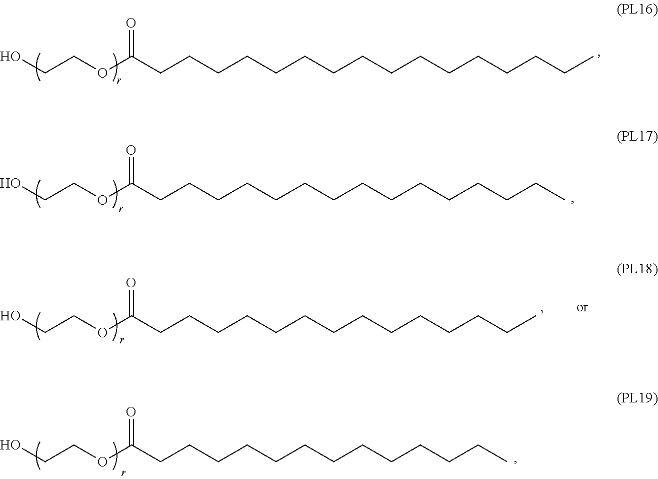

or a pharmaceutically acceptable salt thereof.

In another aspect, provided herein are lipid nanoparticles (LNPs) for the treatment of Parkinson's disease comprising PEG lipids of Formula (PVII):

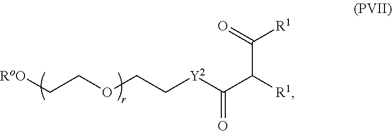

or pharmaceutically acceptable salts thereof, wherein:
Y² is —O—, —NR^{N}—, or —S—
each instance of R¹ is independently optionally substituted C₅₋₃₀ alkyl, optionally substituted C₅₋₃₀ alkenyl, or optionally substituted C₅₋₃₀ alkynyl;
R^{O} is hydrogen, optionally substituted alkyl, optionally substituted acyl, or an oxygen protecting group;
R^{N} is hydrogen, optionally substituted alkyl, optionally substituted acyl, or a nitrogen protecting group; and
r is an integer from 2 to 100, inclusive.

In certain embodiments, the PEG lipid of Formula (PVII) is of one of the following formulae:

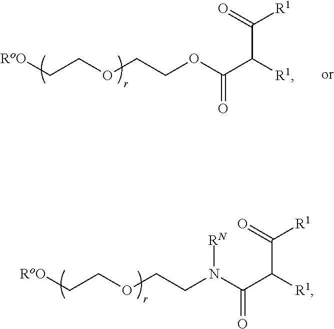

or a pharmaceutically acceptable salt thereof.

In certain embodiments, the PEG lipid of Formula (PVII) is of one of the following formulae:

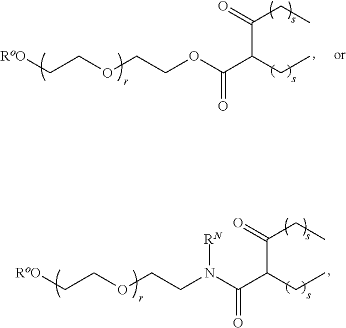

or a pharmaceutically acceptable salt thereof, wherein:
each instance of s is independently an integer from 5-25, inclusive.

In certain embodiments, the PEG lipid of Formula (PVII) is of one of the following formulae:

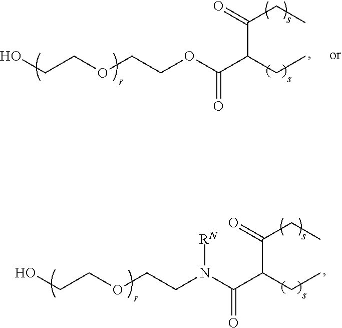

or a pharmaceutically acceptable salt thereof.

In certain embodiments, the PEG lipid of Formula (PVII) is selected from the group consisting of

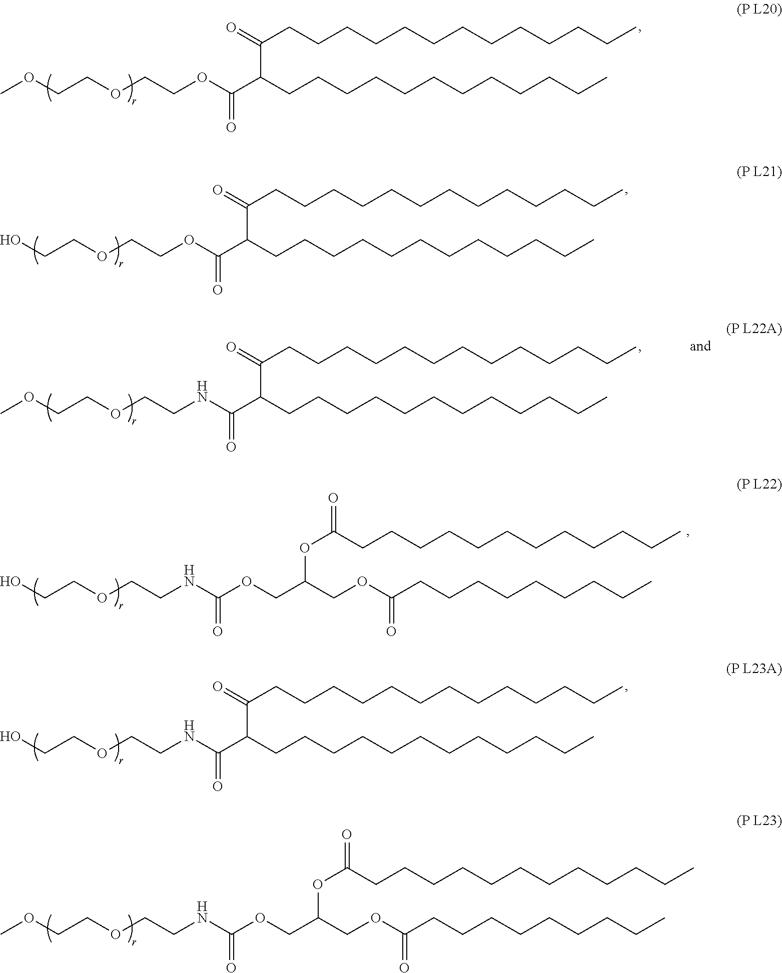

and pharmaceutically acceptable salts thereof.

In another aspect, provided herein are lipid nanoparticles (LNPs) for the treatment of Parkinson's disease comprising PEG lipids of Formula (PVIII):

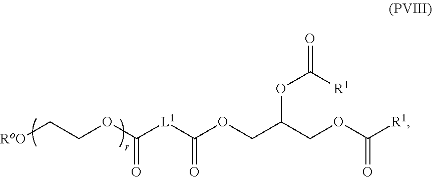

or pharmaceutically acceptable salts thereof, wherein:
L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;
each instance of R¹ is independently optionally substituted C₅₋₃₀ alkyl, optionally substituted C₃₋₃₀ alkenyl, or optionally substituted C₅₋₃₀ alkynyl;
R^{O} is hydrogen, optionally substituted alkyl, optionally substituted acyl, or an oxygen protecting group;
r is an integer from 2 to 100, inclusive;
provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

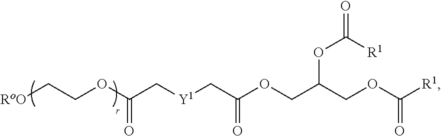

or a pharmaceutically acceptable salt thereof, wherein:
Y¹ is a bond, —CR₂—, —O—, —NR^{N}—, or —S—;
each instance of R is independently hydrogen, halogen, or optionally substituted alkyl;
R^{N} is hydrogen, optionally substituted alkyl, optionally substituted acyl, or a nitrogen protecting group;
provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

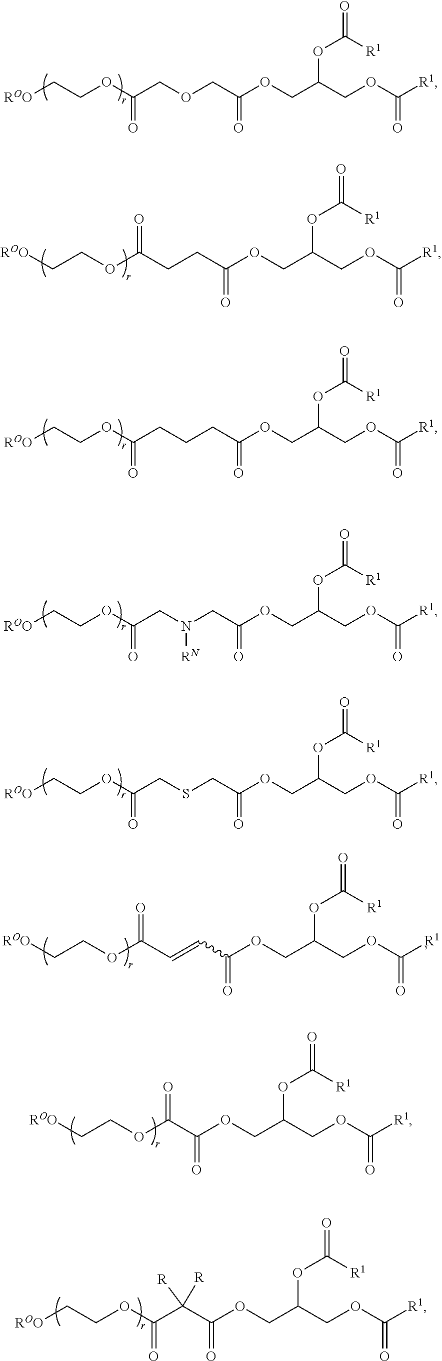

or a pharmaceutically acceptable salt thereof, wherein:
each instance of R is independently hydrogen, halogen, or optionally substituted alkyl.

In certain embodiments, the PEG lipid of Formula (PVIII) is of one of the following formulae:

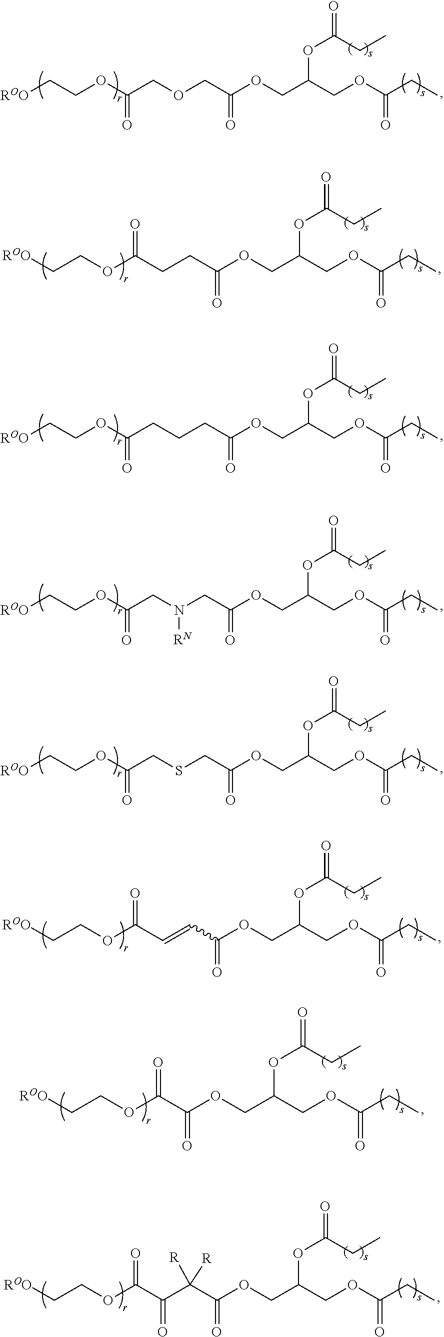

or a pharmaceutically acceptable salt thereof, wherein:
each instance of R is independently hydrogen, halogen, or optionally substituted alkyl; and
each s is independently an integer from 5-25, inclusive.

In certain embodiments, the PEG lipid of Formula (PVIII) is of one of the following formulae:

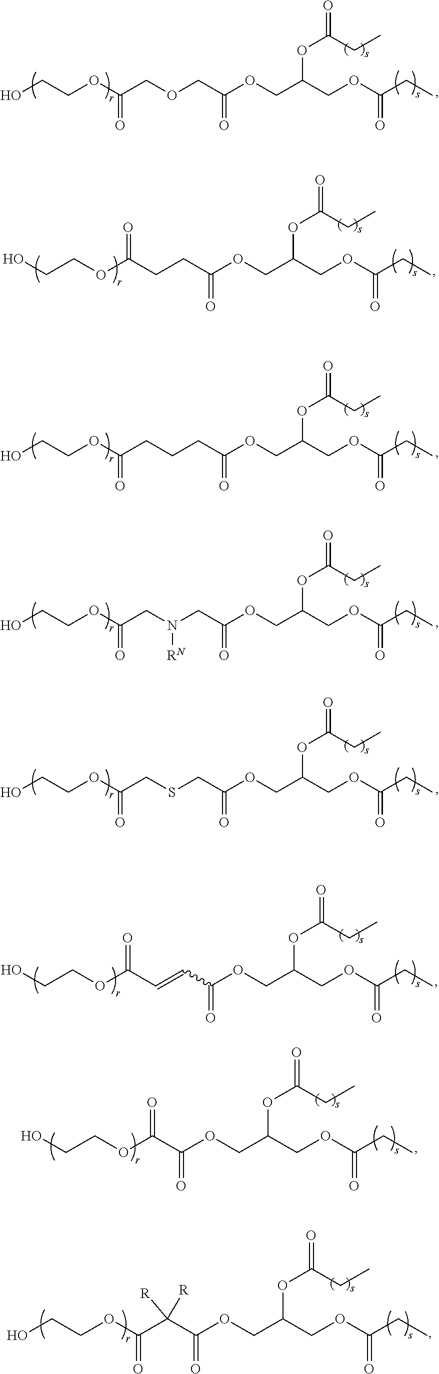

or a pharmaceutically acceptable salt thereof.

In certain embodiments, the PEG lipid of Formula (PVIII) is selected from the group consisting of

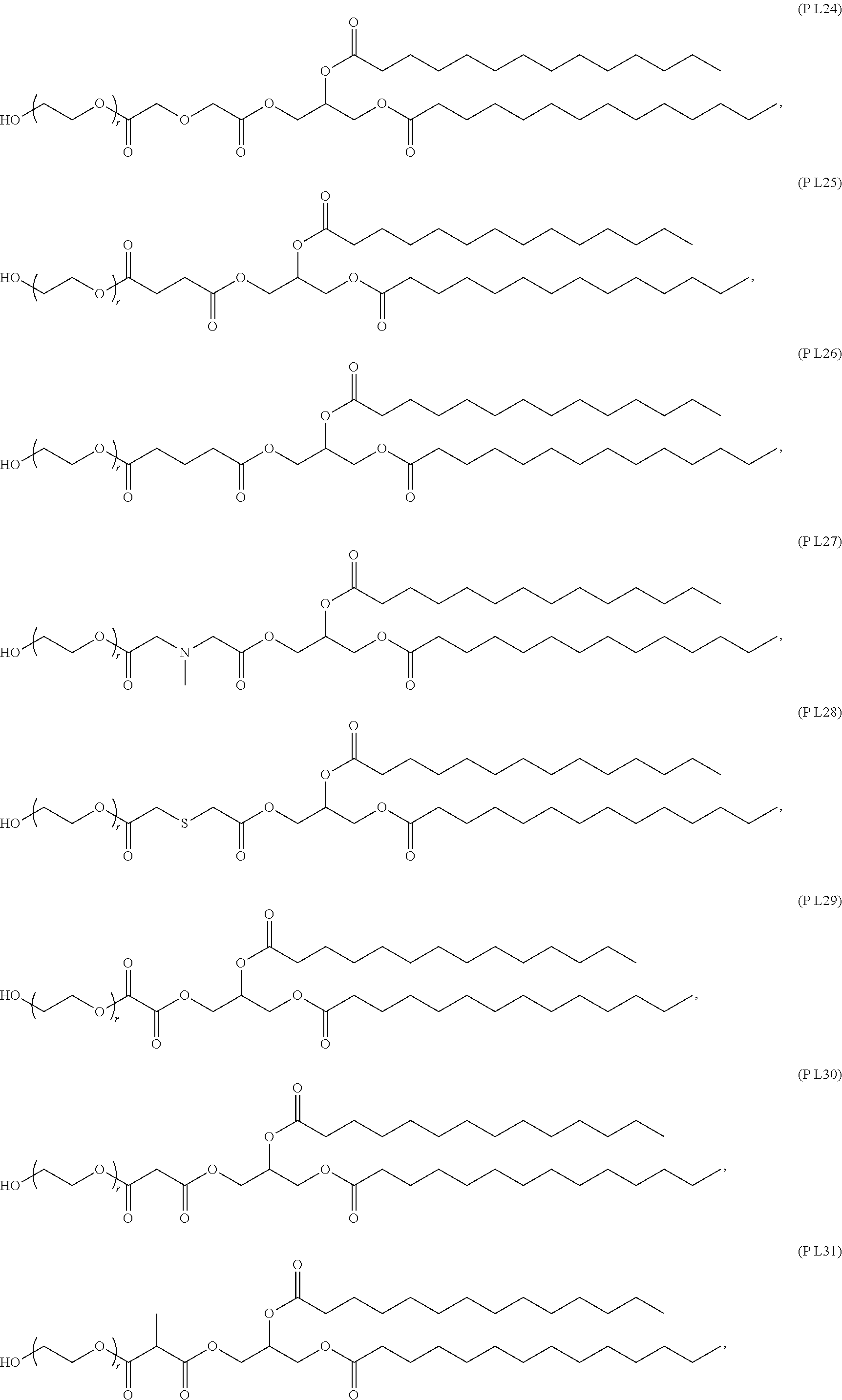

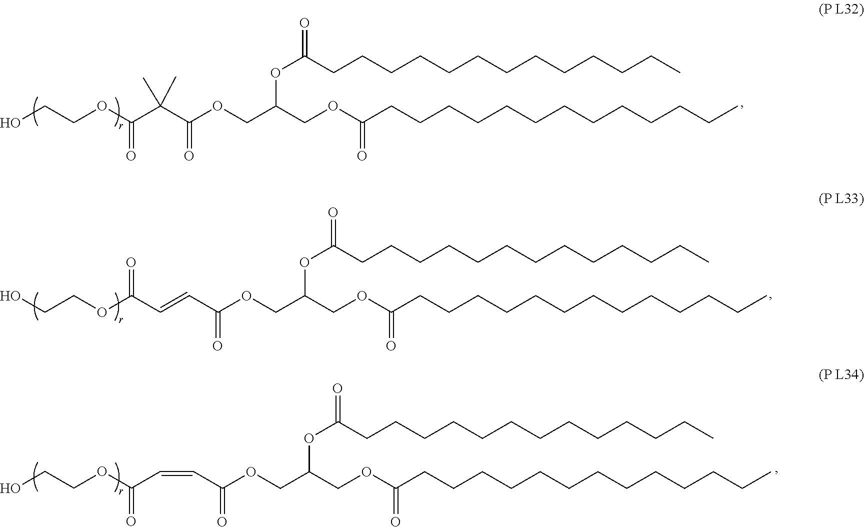

and pharmaceutically acceptable salts thereof.

In any of the foregoing or related aspects, a PEG lipid of the invention is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

Exemplary Synthesis:

Compound: HO-PEG₂₀₀₀-ester-C18

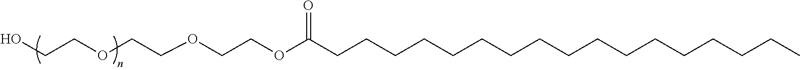

To a nitrogen filled flask containing palladium on carbon (10 wt. %, 74 mg, 0.070 mmol) was added Benzyl-PEG₂₀₀₀-ester-C18 (822 mg, 0.35 mmol) and MeOH (20 mL). The flask was evacuated nad backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, a target cell delivery lipid of the pharmaceutical compositions disclosed herein does not comprise a PEG-lipid.

In one embodiment, a target cell target cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, a target cell target cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of Cmpd 428, PL16, PL17, PL 18, PL19, PL 1, and PL 2.

### Methods of Using the LNP Compositions in the Treatment of Parkinson's Disease and Optimization of Constructs for Use in Such Treatment Methods

In an aspect, the disclosure provides a composition comprising a polynucleotide encoding a human GM-CSF polypeptide for use, in the treatment of Parkinson's disease (PD) in a subject. In an embodiment, the GM-CSF polypeptide comprises the amino acid sequence of SEQ ID NO: 1.

In a related aspect, provided herein is a method of treating Parkinson's disease (PD) in a subject, comprising administering to the subject an effective amount of a lipid nanoparticle (LNP) composition comprising a polynucleotide encoding a human GM-CSF polypeptide. In an embodiment, the GM-CSF polypeptide comprises the amino acid sequence of SEQ ID NO: 1.

In an embodiment of any of the methods of treatment or compositions for use disclosed herein, the subject has, or is identified as having, PD.

In an embodiment, the subject is a mammal, e.g., a human, mouse, or rat. In an embodiment, the subject is a human.

### Sequence Optimization and Methods Thereof

In some embodiments, a polynucleotide of the disclosure comprises a sequence-optimized nucleotide sequence encoding a polypeptide disclosed herein, e.g., GM-CSF. In some embodiments, the polynucleotide of the disclosure comprises an open reading frame (ORF) encoding a GM-CSF polypeptide, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding a GM-CSF polypeptide) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine is present instead of uracil, and one would substitute T where U appears. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding a GM-CSF polypeptide of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding a GM-CSF polypeptide of the disclosure has a reduced number of uracil triplets (UUU) with respect to the wild-type nucleic acid sequence. In some embodiments, a uracil-modified sequence encoding a GM-CSF polypeptide has a reduced number of uracil pairs (UU) with respect to the number of uracil pairs (UU) in the wild-type nucleic acid sequence. In some embodiments, a uracil-modified sequence encoding a GM-CSF polypeptide of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding a GM-CSF polypeptide has a % UUwt between below 100%.

In some embodiments, the polynucleotide of the disclosure comprises a uracil-modified sequence encoding a GM-CSF polypeptide disclosed herein. In some embodiments, the uracil-modified sequence encoding a GM-CSF polypeptide comprises at least one chemically modified nucleobase, e.g., 5-methoxyuracil. In some embodiments, at least 95% of a nucleobase (e.g., uracil) in a uracil-modified sequence encoding a GM-CSF polypeptide of the disclosure are modified nucleobases. In some embodiments, at least 95% of uracil in a uracil-modified sequence encoding a GM-CSF polypeptide is 5-methoxyuracil. In some embodiments, the polynucleotide comprising a uracil-modified sequence further comprises a miRNA binding site, e.g., a miRNA binding site that binds to miR-122. In some embodiments, the polynucleotide comprising a uracil-modified sequence is formulated with a delivery agent, e.g., a compound having Formula (I), e.g., any of Compounds 1-147, or any of Compounds 1-232.

In some embodiments, a polynucleotide of the disclosure (e.g., a polynucleotide comprising a nucleotide sequence encoding a GM-CSF polypeptide (e.g., the wild-type sequence, functional fragment, or variant thereof)) is sequence optimized.

A sequence optimized nucleotide sequence (nucleotide sequence is also referred to as “nucleic acid” herein) comprises at least one codon modification with respect to a reference sequence (e.g., a wild-type sequence encoding a GM-CSF polypeptide). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence-optimized nucleic acids (e.g., a RNA, e.g., an mRNA) can be administered to a subject in need thereof to facilitate in vivo expression of functionally active GM-CSF polypeptide.

Additional and exemplary methods of sequence optimization are disclosed in International PCT application WO 2017/201325, filed on 18 May 2017, the entire contents of which are hereby incorporated by reference.

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

The present invention also provides pharmaceutical compositions and formulations that comprise any of the polynucleotides described above. In some embodiments, the composition or formulation further comprises a delivery agent.

In some embodiments, the composition or formulation can contain a polynucleotide comprising a sequence optimized nucleic acid sequence disclosed herein which encodes a polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a polypeptide. In some embodiments, the polynucleotide further comprises a miRNA binding site, e.g., a miRNA binding site that binds

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA.

microRNAs derive enzymatically from regions of RNA transcripts that fold back on themselves to form short hairpin structures often termed a pre-miRNA (precursor-miRNA). A pre-miRNA typically has a two-nucleotide overhang at its 3′ end, and has 3′ hydroxyl and 5′ phosphate groups. This precursor-mRNA is processed in the nucleus and subsequently transported to the cytoplasm where it is further processed by DICER (a RNase III enzyme), to form a mature microRNA of approximately 22 nucleotides. The mature microRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives; “5p” means the microRNA is from the 5-prime arm of the pre-miRNA hairpin and “3p” means the microRNA is from the 3-prime end of the pre-miRNA hairpin. A miR referred to by number herein can refer to either of the two mature microRNAs originating from opposite arms of the same pre-miRNA (e.g., either the 3p or 5p microRNA). All miRs referred to herein are intended to include both the 3p and 5p arms/sequences, unless particularly specified by the 3p or 5p designation. As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′ UTR and/or 3′ UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide long miRNA sequence, to a 19-23 nucleotide long miRNA sequence, or to a 22-nucleotide long miRNA sequence. A miRNA binding site can be complementary to only a portion of a miRNA, e.g., to a portion less than 1, 2, 3, or 4 nucleotides of the full length of a naturally-occurring miRNA sequence, or to a portion less than 1, 2, 3, or 4 nucleotides shorter than a naturally-occurring miRNA sequence. Full or complete complementarity (e.g., full complementarity or complete complementarity over all or a significant portion of the length of a naturally-occurring miRNA) is preferred when the desired regulation is mRNA degradation. In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the polynucleotide. Thus, in some embodiments, incorporation of one or more miRNA binding sites into an mRNA of the disclosure may reduce the hazard of off-target effects upon nucleic acid molecule delivery and/or enable tissue-specific regulation of expression of a polypeptide encoded by the mRNA. In yet other embodiments, incorporation of one or more miRNA binding sites into an mRNA of the disclosure can modulate immune responses upon nucleic acid delivery in vivo. In further embodiments, incorporation of one or more miRNA binding sites into an mRNA of the disclosure can modulate accelerated blood clearance (ABC) of lipid-comprising compounds and compositions described herein.

Conversely, miRNA binding sites can be removed from polynucleotide sequences in which they naturally occur to increase protein expression in specific tissues. For example, a binding site for a specific miRNA can be removed from a polynucleotide to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′ UTR and/or 3′UTR of a polynucleotide of the invention can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In one embodiment, binding sites for miRNAs that are known to be expressed in immune cells, in particular, antigen presenting cells, can be engineered into a polynucleotide of the invention to suppress the expression of the polynucleotide in antigen presenting cells through miRNA mediated RNA degradation, subduing the antigen-mediated immune response. Expression of the polynucleotide is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′ UTR and/or 3′ UTR of a polynucleotide of the invention.

In some embodiments, the polynucleotide of the invention can include a further negative regulatory element in the 5′ UTR and/or 3′ UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11, 288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a polynucleotide of the invention to regulate expression of the polynucleotide in the liver. Liver specific miRNA binding sites can be engineered alone or further in combination with immune cell (e.g., APC) miRNA binding sites in a polynucleotide of the invention.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a polynucleotide of the invention to regulate expression of the polynucleotide in the lung. Lung specific miRNA binding sites can be engineered alone or further in combination with immune cell (e.g., APC) miRNA binding sites in a polynucleotide of the invention.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. miRNA binding sites from any heart specific microRNA can be introduced to or removed from a polynucleotide of the invention to regulate expression of the polynucleotide in the heart. Heart specific miRNA binding sites can be engineered alone or further in combination with immune cell (e.g., APC) miRNA binding sites in a polynucleotide of the invention.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a polynucleotide of the invention to regulate expression of the polynucleotide in the nervous system. Nervous system specific miRNA binding sites can be engineered alone or further in combination with immune cell (e.g., APC) miRNA binding sites in a polynucleotide of the invention.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a polynucleotide of the invention to regulate expression of the polynucleotide in the pancreas. Pancreas specific miRNA binding sites can be engineered alone or further in combination with immune cell (e.g. APC) miRNA binding sites in a polynucleotide of the invention.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a polynucleotide of the invention to regulate expression of the polynucleotide in the kidney. Kidney specific miRNA binding sites can be engineered alone or further in combination with immune cell (e.g., APC) miRNA binding sites in a polynucleotide of the invention.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA binding sites from any muscle specific miRNA can be introduced to or removed from a polynucleotide of the invention to regulate expression of the polynucleotide in the muscle. Muscle specific miRNA binding sites can be engineered alone or further in combination with immune cell (e.g., APC) miRNA binding sites in a polynucleotide of the invention.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a polynucleotide of the invention to regulate expression of the polynucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a polynucleotide of the invention to regulate expression of the polynucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-548l, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941,miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In some embodiments, miRNAs are selected based on expression and abundance in immune cells of the hematopoietic lineage, such as B cells, T cells, macrophages, dendritic cells, and cells that are known to express TLR7/TLR8 and/or able to secrete cytokines such as endothelial cells and platelets. In some embodiments, the miRNA set thus includes miRs that may be responsible in part for the immunogenicity of these cells, and such that a corresponding miR-site incorporation in polynucleotides of the present invention (e.g., mRNAs) could lead to destabilization of the mRNA and/or suppression of translation from these mRNAs in the specific cell type. Non-limiting representative examples include miR-142, miR-144, miR-150, miR-155 and miR-223, which are specific for many of the hematopoietic cells; miR-142, miR150, miR-16 and miR-223, which are expressed in B cells; miR-223, miR-451, miR-26a, miR-16, which are expressed in progenitor hematopoietic cells; and miR-126, which is expressed in plasmacytoid dendritic cells, platelets and endothelial cells. For further discussion of tissue expression of miRs see e.g., Teruel-Montoya, R. et al. (2014) PLoS One 9:e102259; Landgraf, P. et al. (2007) Cell 129:1401-1414; Bissels, U. et al. (2009) RNA 15:2375-2384. Any one miR-site incorporation in the 3′ UTR and/or 5′ UTR may mediate such effects in multiple cell types of interest (e.g., miR-142 is abundant in both B cells and dendritic cells).

In some embodiments, it may be beneficial to target the same cell type with multiple miRs and to incorporate binding sites to each of the 3p and 5p arm if both are abundant (e.g., both miR-142-3p and miR142-5p are abundant in hematopoietic stem cells). Thus, in certain embodiments, polynucleotides of the invention contain two or more (e.g., two, three, four or more) miR bindings sites from: (i) the group consisting of miR-142, miR-144, miR-150, miR-155 and miR-223 (which are expressed in many hematopoietic cells); or (ii) the group consisting of miR-142, miR150, miR-16 and miR-223 (which are expressed in B cells); or the group consisting of miR-223, miR-451, miR-26a, miR-16 (which are expressed in progenitor hematopoietic cells).

In some embodiments, it may also be beneficial to combine various miRs such that multiple cell types of interest are targeted at the same time (e.g., miR-142 and miR-126 to target many cells of the hematopoietic lineage and endothelial cells). Thus, for example, in certain embodiments, polynucleotides of the invention comprise two or more (e.g., two, three, four or more) miRNA bindings sites, wherein: (i) at least one of the miRs targets cells of the hematopoietic lineage (e.g., miR-142, miR-144, miR-150, miR-155 or miR-223) and at least one of the miRs targets plasmacytoid dendritic cells, platelets or endothelial cells (e.g., miR-126); or (ii) at least one of the miRs targets B cells (e.g., miR-142, miR150, miR-16 or miR-223) and at least one of the miRs targets plasmacytoid dendritic cells, platelets or endothelial cells (e.g., miR-126); or (iii) at least one of the miRs targets progenitor hematopoietic cells (e.g., miR-223, miR-451, miR-26a or miR-16) and at least one of the miRs targets plasmacytoid dendritic cells, platelets or endothelial cells (e.g., miR-126); or (iv) at least one of the miRs targets cells of the hematopoietic lineage (e.g., miR-142, miR-144, miR-150, miR-155 or miR-223), at least one of the miRs targets B cells (e.g., miR-142, miR150, miR-16 or miR-223) and at least one of the miRs targets plasmacytoid dendritic cells, platelets or endothelial cells (e.g., miR-126); or any other possible combination of the foregoing four classes of miR binding sites (i.e., those targeting the hematopoietic lineage, those targeting B cells, those targeting progenitor hematopoietic cells and/or those targeting plasmacytoid dendritic cells/platelets/endothelial cells).

In one embodiment, to modulate immune responses, polynucleotides of the present invention can comprise one or more miRNA binding sequences that bind to one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells). It has now been discovered that incorporation into an mRNA of one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells) reduces or inhibits immune cell activation (e.g., B cell activation, as measured by frequency of activated B cells) and/or cytokine production (e.g., production of IL-6, IFN-γ and/or TNFα). Furthermore, it has now been discovered that incorporation into an mRNA of one or more miRs that are expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells) can reduce or inhibit an anti-drug antibody (ADA) response against a protein of interest encoded by the mRNA.

In another embodiment, to modulate accelerated blood clearance of a polynucleotide delivered in a lipid-comprising compound or composition, polynucleotides of the invention can comprise one or more miR binding sequences that bind to one or more miRNAs expressed in conventional immune cells or any cell that expresses TLR7 and/or TLR8 and secrete pro-inflammatory cytokines and/or chemokines (e.g., in immune cells of peripheral lymphoid organs and/or splenocytes and/or endothelial cells). It has now been discovered that incorporation into an mRNA of one or more miR binding sites reduces or inhibits accelerated blood clearance (ABC) of the lipid-comprising compound or composition for use in delivering the mRNA.

Furthermore, it has now been discovered that incorporation of one or more miR binding sites into an mRNA reduces serum levels of anti-PEG anti-IgM (e.g., reduces or inhibits the acute production of IgMs that recognize polyethylene glycol (PEG) by B cells) and/or reduces or inhibits proliferation and/or activation of plasmacytoid dendritic cells following administration of a lipid-comprising compound or composition comprising the mRNA.

In some embodiments, miR sequences may correspond to any known microRNA expressed in immune cells, including but not limited to those taught in US Publication US2005/0261218 and US Publication US2005/0059005, the contents of which are incorporated herein by reference in their entirety. Non-limiting examples of miRs expressed in immune cells include those expressed in spleen cells, myeloid cells, dendritic cells, plasmacytoid dendritic cells, B cells, T cells and/or macrophages. For example, miR-142-3p, miR-142-5p, miR-16, miR-21, miR-223, miR-24 and miR-27 are expressed in myeloid cells, miR-155 is expressed in dendritic cells, B cells and T cells, miR-146 is upregulated in macrophages upon TLR stimulation and miR-126 is expressed in plasmacytoid dendritic cells. In certain embodiments, the miR(s) is expressed abundantly or preferentially in immune cells. For example, miR-142 (miR-142-3p and/or miR-142-5p), miR-126 (miR-126-3p and/or miR-126-5p), miR-146 (miR-146-3p and/or miR-146-5p) and miR-155 (miR-155-3p and/or miR155-5p) are expressed abundantly in immune cells. These microRNA sequences are known in the art and, thus, one of ordinary skill in the art can readily design binding sequences or target sequences to which these microRNAs will bind based upon Watson-Crick complementarity.

Accordingly, in various embodiments, polynucleotides of the present invention comprise at least one microRNA binding site for a miR selected from the group consisting of miR-142, miR-146, miR-155, miR-126, miR-16, miR-21, miR-223, miR-24 and miR-27. In another embodiment, the mRNA comprises at least two miR binding sites for microRNAs expressed in immune cells. In various embodiments, the polynucleotide of the invention comprises 1-4, one, two, three or four miR binding sites for microRNAs expressed in immune cells. In another embodiment, the polynucleotide of the invention comprises three miR binding sites. These miR binding sites can be for microRNAs selected from the group consisting of miR-142, miR-146, miR-155, miR-126, miR-16, miR-21, miR-223, miR-24, miR-27, and combinations thereof. In one embodiment, the polynucleotide of the invention comprises two or more (e.g., two, three, four) copies of the same miR binding site expressed in immune cells, e.g., two or more copies of a miR binding site selected from the group of miRs consisting of miR-142, miR-146, miR-155, miR-126, miR-16, miR-21, miR-223, miR-24, miR-27.

In one embodiment, the polynucleotide of the invention comprises three copies of the same miRNA binding site. In certain embodiments, use of three copies of the same miR binding site can exhibit beneficial properties as compared to use of a single miRNA binding site. Non-limiting examples of sequences for 3′ UTRs containing three miRNA bindings sites are shown in SEQ ID NO: 155 (three miR-142-3p binding sites) and SEQ ID NO: 157 (three miR-142-5p binding sites).

In another embodiment, the polynucleotide of the invention comprises two or more (e.g., two, three, four) copies of at least two different miR binding sites expressed in immune cells. Non-limiting examples of sequences of 3′ UTRs containing two or more different miR binding sites are shown in SEQ ID NO:111 (one miR-142-3p binding site and one miR-126-3p binding site), SEQ ID NO: 158 (two miR-142-5p binding sites and one miR-142-3p binding sites), and SEQ ID NO: 161 (two miR-155-5p binding sites and one miR-142-3p binding sites).

In another embodiment, the polynucleotide of the invention comprises at least two miR binding sites for microRNAs expressed in immune cells, wherein one of the miR binding sites is for miR-142-3p. In various embodiments, the polynucleotide of the invention comprises binding sites for miR-142-3p and miR-155 (miR-155-3p or miR-155-5p), miR-142-3p and miR-146 (miR-146-3 or miR-146-5p), or miR-142-3p and miR-126 (miR-126-3p or miR-126-5p).

In another embodiment, the polynucleotide of the invention comprises at least two miR binding sites for microRNAs expressed in immune cells, wherein one of the miR binding sites is for miR-126-3p. In various embodiments, the polynucleotide of the invention comprises binding sites for miR-126-3p and miR-155 (miR-155-3p or miR-155-5p), miR-126-3p and miR-146 (miR-146-3p or miR-146-5p), or miR-126-3p and miR-142 (miR-142-3p or miR-142-5p).

In another embodiment, the polynucleotide of the invention comprises at least two miR binding sites for microRNAs expressed in immune cells, wherein one of the miR binding sites is for miR-142-5p. In various embodiments, the polynucleotide of the invention comprises binding sites for miR-142-5p and miR-155 (miR-155-3p or miR-155-5p), miR-142-5p and miR-146 (miR-146-3 or miR-146-5p), or miR-142-5p and miR-126 (miR-126-3p or miR-126-5p).

In yet another embodiment, the polynucleotide of the invention comprises at least two miR binding sites for microRNAs expressed in immune cells, wherein one of the miR binding sites is for miR-155-5p. In various embodiments, the polynucleotide of the invention comprises binding sites for miR-155-5p and miR-142 (miR-142-3p or miR-142-5p), miR-155-5p and miR-146 (miR-146-3 or miR-146-5p), or miR-155-5p and miR-126 (miR-126-3p or miR-126-5p). miRNA can also regulate complex biological processes such as angiogenesis (e.g., miR-132) (Anand and Cheresh Curr Opin Hematol 2011 18:171-176). In the polynucleotides of the invention, miRNA binding sites that are involved in such processes can be removed or introduced, to tailor the expression of the polynucleotides to biologically relevant cell types or relevant biological processes. In this context, the polynucleotides of the invention are defined as auxotrophic polynucleotides.

In some embodiments, a polynucleotide of the invention comprises a miRNA binding site, wherein the miRNA binding site comprises one or more nucleotide sequences selected from Table 3C or Table 4B, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from Table 3C or Table 4B, including any combination thereof.

In some embodiments, the miRNA binding site binds to miR-142 or is complementary to miR-142. In some embodiments, the miR-142 comprises SEQ ID NO: 114. In some embodiments, the miRNA binding site binds to miR-142-3p or miR-142-5p. In some embodiments, the miR-142-3p binding site comprises SEQ ID NO:116. In some embodiments, the miR-142-5p binding site comprises SEQ ID NO:118. In some embodiments, the miRNA binding site comprises a nucleotide sequence at least 80%, at least 85%, at least 90%, at least 95%, or 100% identical to SEQ ID NO:116 or SEQ ID NO:118.

In some embodiments, the miRNA binding site binds to miR-126 or is complementary to miR-126. In some embodiments, the miR-126 comprises SEQ ID NO: 119. In some embodiments, the miRNA binding site binds to miR-126-3p or miR-126-5p. In some embodiments, the miR-126-3p binding site comprises SEQ ID NO: 121. In some embodiments, the miR-126-5p binding site comprises SEQ ID NO: 123. In some embodiments, the miRNA binding site comprises a nucleotide sequence at least 80%, at least 85%, at least 90%, at least 95%, or 100% identical to SEQ ID NO: 121 or SEQ ID NO: 123.

In one embodiment, the 3′ UTR comprises two miRNA binding sites, wherein a first miRNA binding site binds to miR-142 and a second miRNA binding site binds to miR-126. In a specific embodiment, the 3′ UTR binding to miR-142 and miR-126 comprises, consists, or consists essentially of the sequence of SEQ ID NO: 163.

TABLE 3C
miR-142, miR-126, and miR-142 and miR-126 binding sites

| SEQ ID NO. | Description | Sequence |
|---|---|---|
| 114 | miR-142 | GACAGUGCAGUCACCCAUAAAGUAGA |
| | | AAGCACUACUAACAGCACUGGAGGGU |
| | | GUAGUGUUUCCUACUUUAUGGAUGAG |
| | | UGUACUGUG |
| 115 | miR-142-3p | uguaguguuuccuacuuuaugga |
| 116 | miR-142-3p binding | uccauaaaguaggaaacacuaca |
| | site | |
| 117 | miR-142-5p | cauaaaguagaaagcacuacu |
| 118 | miR-142-5p binding | aguagugcuuucuacuuuaug |
| | site | |
| 119 | miR-126 | CGCUGGCGACGGGACAUUAUUACUUU |
| | | UGGUACGCGCUGUGACACUUCAAACU |
| | | CGUACCGUGAGUAAUAAUGCGCCGUC |
| | | CACGGCA |
| 120 | miR-126-3p | UCGUACCGUGAGUAAUAAUGCG |
| 121 | miR-126-3p binding | CGCAUUAUUACUCACGGUACGA |
| | site | |
| 122 | miR-126-5p | CAUUAUUACUUUUGGUACGCG |
| 123 | miR-126-5p binding | CGCGUACCAAAAGUAAUAAUG |
| | site | |

In some embodiments, a miRNA binding site is inserted in the polynucleotide of the invention in any position of the polynucleotide (e.g., the 5′ UTR and/or 3′ UTR). In some embodiments, the 5′ UTR comprises a miRNA binding site. In some embodiments, the 3′ UTR comprises a miRNA binding site. In some embodiments, the 5′ UTR and the 3′ UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide. In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention.

In some embodiments, a miRNA binding site is inserted within the 3′ UTR immediately following the stop codon of the coding region within the polynucleotide of the invention, e.g., mRNA. In some embodiments, if there are multiple copies of a stop codon in the construct, a miRNA binding site is inserted immediately following the final stop codon. In some embodiments, a miRNA binding site is inserted further downstream of the stop codon, in which case there are 3′ UTR bases between the stop codon and the miR binding site(s). In some embodiments, three non-limiting examples of possible insertion sites for a miR in a 3′ UTR are shown in SEQ ID NOs: 162, 163, and 164, which show a 3′ UTR sequence with a miR-142-3p site inserted in one of three different possible insertion sites, respectively, within the 3′ UTR. In some embodiments, one or more miRNA binding sites can be positioned within the 5′ UTR at one or more possible insertion sites. For example, three non-limiting examples of possible insertion sites for a miR in a 5′ UTR are shown in SEQ ID NOs: 165, 166, or 167, which show a 5′ UTR sequence with a miR-142-3p site inserted into one of three different possible insertion sites, respectively, within the 5′ UTR.

In one embodiment, a codon optimized open reading frame encoding a polypeptide of interest comprises a stop codon and the at least one microRNA binding site is located within the 3′ UTR 1-100 nucleotides after the stop codon. In one embodiment, the codon optimized open reading frame encoding the polypeptide of interest comprises a stop codon and the at least one microRNA binding site for a miR expressed in immune cells is located within the 3′ UTR 30-50 nucleotides after the stop codon. In another embodiment, the codon optimized open reading frame encoding the polypeptide of interest comprises a stop codon and the at least one microRNA binding site for a miR expressed in immune cells is located within the 3′ UTR at least 50 nucleotides after the stop codon. In other embodiments, the codon optimized open reading frame encoding the polypeptide of interest comprises a stop codon and the at least one microRNA binding site for a miR expressed in immune cells is located within the 3′ UTR immediately after the stop codon, or within the 3′ UTR 15-20 nucleotides after the stop codon or within the 3′ UTR 70-80 nucleotides after the stop codon. In other embodiments, the 3′ UTR comprises more than one miRNA binding site (e.g., 2-4 miRNA binding sites), wherein there can be a spacer region (e.g., of 10-100, 20-70 or 30-50 nucleotides in length) between each miRNA binding site. In another embodiment, the 3′ UTR comprises a spacer region between the end of the miRNA binding site(s) and the poly A tail nucleotides. For example, a spacer region of 10-100, 20-70 or 30-50 nucleotides in length can be situated between the end of the miRNA binding site(s) and the beginning of the poly A tail.

In one embodiment, a codon optimized open reading frame encoding a polypeptide of interest comprises a start codon and the at least one microRNA binding site is located within the 5′ UTR 1-100 nucleotides before (upstream of) the start codon. In one embodiment, the codon optimized open reading frame encoding the polypeptide of interest comprises a start codon and the at least one microRNA binding site for a miR expressed in immune cells is located within the 5′ UTR 10-50 nucleotides before (upstream of) the start codon. In another embodiment, the codon optimized open reading frame encoding the polypeptide of interest comprises a start codon and the at least one microRNA binding site for a miR expressed in immune cells is located within the 5′ UTR at least 25 nucleotides before (upstream of) the start codon. In other embodiments, the codon optimized open reading frame encoding the polypeptide of interest comprises a start codon and the at least one microRNA binding site for a miR expressed in immune cells is located within the 5′ UTR immediately before the start codon, or within the 5′ UTR 15-20 nucleotides before the start codon or within the 5′ UTR 70-80 nucleotides before the start codon. In other embodiments, the 5′ UTR comprises more than one miRNA binding site (e.g., 2-4 miRNA binding sites), wherein there can be a spacer region (e.g., of 10-100, 20-70 or 30-50 nucleotides in length) between each miRNA binding site.

In one embodiment, the 3′ UTR comprises more than one stop codon, wherein at least one miRNA binding site is positioned downstream of the stop codons. For example, a 3′ UTR can comprise 1, 2 or 3 stop codons. Non-limiting examples of triple stop codons that can be used include: UGAUAAUAG (SEQ ID NO:124), UGAUAGUAA (SEQ ID NO:125), UAAUGAUAG (SEQ ID NO:126), UGAUAAUAA (SEQ ID NO:127), UGAUAGUAG (SEQ ID NO:128), UAAUGAUGA (SEQ ID NO:129), UAAUAGUAG (SEQ ID NO:130), UGAUGAUGA (SEQ ID NO:131), UAAUAAUAA (SEQ ID NO:132), and UAGUAGUAG (SEQ ID NO:133). Within a 3′ UTR, for example, 1, 2, 3 or 4 miRNA binding sites, e.g., miR-142-3p binding sites, can be positioned immediately adjacent to the stop codon(s) or at any number of nucleotides downstream of the final stop codon. When the 3′ UTR comprises multiple miRNA binding sites, these binding sites can be positioned directly next to each other in the construct (i.e., one after the other) or, alternatively, spacer nucleotides can be positioned between each binding site.

In one embodiment, the 3′ UTR comprises three stop codons with a single miR-142-3p binding site located downstream of the 3rd stop codon. Non-limiting examples of sequences of 3′ UTR having three stop codons and a single miR-142-3p binding site located at different positions downstream of the final stop codon are shown in SEQ ID NOs: 151, 162, 163, and 164.

TABLE 4B
5′ UTRs, 3′UTRs, miR sequences, and miR binding sites

| SEQ ID NO: | Sequence |
|---|---|
| 134 | GCUGGAGCCUCGGUGGCCAUGCUUCUUGCCCCUUGGGCCUCCCCCCAGCCCC |
| | UCCUCCCCUUCCUGCACCCGUACCCCCUCCAUAAAGUAGGAAACACUACAGU |
| | GGUCUUUGAAUAAAGUCUGAGUGGGGGC |
| | (3′ UTR with miR 142-3p binding site) |
| 116 | UCCAUAAAGUAGGAAACACUACA |
| | (miR 142-3p binding site) |
| 115 | UGUAGUGUUUCCUACUUUAUGGA |
| | (miR 142-3p sequence) |
| 117 | CAUAAAGUAGAAAGCACUACU |
| | (miR 142-5p sequence) |
| 135 | CCUCUGAAAUUCAGUUCUUCAG |
| | (miR 146-3p sequence) |
| 136 | UGAGAACUGAAUUCCAUGGGUU |
| | (miR 146-5p sequence) |
| 137 | CUCCUACAUAUUAGCAUUAACA |
| | (miR 155-3p sequence) |
| 138 | UUAAUGCUAAUCGUGAUAGGGGU |
| | (miR 155-5p sequence) |
| 120 | UCGUACCGUGAGUAAUAAUGCG |
| | (miR 126-3p sequence) |
| 122 | CAUUAUUACUUUUGGUACGCG |
| | (miR 126-5p sequence) |
| 139 | CCAGUAUUAACUGUGCUGCUGA |
| | (miR 16-3p sequence) |
| 140 | UAGCAGCACGUAAAUAUUGGCG |
| | (miR 16-5p sequence) |
| 141 | CAACACCAGUCGAUGGGCUGU |
| | (miR 21-3p sequence) |
| 142 | UAGCUUAUCAGACUGAUGUUGA |
| | (miR 21-5p sequence) |
| 143 | UGUCAGUUUGUCAAAUACCCCA |
| | (miR 223-3p sequence) |
| 144 | CGUGUAUUUGACAAGCUGAGUU |
| | (miR 223-5p sequence) |
| 145 | UGGCUCAGUUCAGCAGGAACAG |
| | (miR 24-3p sequence) |
| 146 | UGCCUACUGAGCUGAUAUCAGU |
| | (miR 24-5p sequence) |
| 147 | UUCACAGUGGCUAAGUUCCGC |
| | (miR 27-3p sequence) |
| 148 | AGGGCUUAGCUGCUUGUGAGCA |
| | (miR 27-5p sequence) |
| 121 | CGCAUUAUUACUCACGGUACGA |
| | (miR 126-3p binding site) |
| 149 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCC [symbol] |
| | [symbol] GUGGUCUUUGAAUAAAGUCUGAGUGGGGGC |
| | (3′ UTR with miR 126-3p binding site) |
| 150 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCGUGGUCUUUGAAUAAA |
| | GUCUGAGUGGGCGGC |
| | (3′ UTR, no miR binding sites) |
| 151 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCUCCAUAAAGUAGGAAA |
| | CACUACAGUGGUCUUUGAAUAAAGUCUGAGUGGGCGGC |
| | (3′ UTR with miR 142-3p binding site) |
| 111 | UGAUAAUAGUCCAUAAAGUAGGAAACACUACAGCUGGAGCCUCGGUGGCCAU |
| | GCUUCUUGCCCCUUGGGCCUCCCCCCAGCCCCUCCUCCCCUUCCUGCACCCG |
| | UACCCCC [symbol] GUGGUCUUUGAAUAAAGUCUGAG |
| | UGGGCGGC |
| | (3′ UTR with miR 142-3p and miR 126-3p binding sites variant 1) |
| 153 | UUAAUGCUAAUUGUGAUAGGGGU |
| | (miR 155-5p sequence) |
| 154 | ACCCCUAUCACAAUUAGCAUUAA |
| | (miR 155-5p binding site) |
| 155 | UGAUAAUAGUCCAUAAAGUAGGAAACACUACAGCUGGAGCCUCGGUGGCCAU |
| | GCUUCUUGCCCCUUGGGCCUCCAUAAAGUAGGAAACACUACAUCCCCCCAGC |
| | CCCUCCUCCCCUUCCUGCACCCGUACCCCCUCCAUAAAGUAGGAAACACUAC |
| | AGUGGUCUUUGAAUAAAGUCUGAGUGGGCGGC |
| | (3′ UTR with 3 miR 142-3p binding sites) |
| 156 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-5p binding site) |
| 157 | UGAUAAUAG [symbol] GCUGGAGCCUCGGUGGCCAUGC |
| | UUCUUGCCCCUUGGGCC [symbol] UCCCCCCAGCCCCU |
| | CCUCCCCUUCCUGCACCCGUACCCCC [symbol] GUGGU |
| | CUUUGAAUAAAGUCUGAGUGGGCGGC |
| | (3′ UTR with 3 miR 142-5p binding sites) |
| 158 | UGAUAAUAG [symbol] GCUGGAGCCUCGGUGGCCAUGC |
| | UUCUUGCCCCUUGGGCCUCCAUAAAGUAGGAAACACUACAUCCCCCCAGCCC |
| | CUCCUCCCCUUCCUGCACCCGUACCCCC [symbol] GUG |
| | GUCUUUGAAUAAAGUCUGAGUGGGCGGC |
| | (3′ UTR with 2 miR 142-5p binding sites and 1 miR 142-3p binding site) |
| 159 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCACCCCUAUCACAAUUA |
| | GCAUUAAGUGGUCUUUGAAUAAAGUCUGAGUGGGCGGC |
| | (3′ UTR with miR 155-5p binding site) |
| 160 | UGAUAAUAGACCCCUAUCACAAUUAGCAUUAAGCUGGAGCCUCGGUGGCCAU |
| | GCUUCUUGCCCCUUGGGCCACCCCUAUCACAAUUAGCAUUAAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 3 miR 155-5p binding sites) |
| 161 | UGAUAAUAGACCCCUAUCACAAUUAGCAUUAAGCUGGAGCCUCGGUGGCCAU |
| | GCUUCUUGCCCCUUGGGCCUCCAUAAAGUAGGAAACACUACAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 2 miR 155-5p binding sites and 1 miR 142-3p binding site) |
| 162 | UGAUAAUAGUCCAUAAAGUAGGAAACACUACAGCUGGAGCCUCGGUGGCCAU |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P1 insertion) |
| 163 | UGAUAAUAGGCUGGAGCCUCGGUGGCUCCAUAAAGUAGGAAACACUACACAU |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P2 insertion) |
| 164 | UGAUAAUAGGCUGGAGCCUCGGUGGCCAUGCUUCUUGCCCCUUGGGCCUCCA |
| | UAAAGUAGGAAACACUACAUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P3 insertion) |
| 118 | AGUAGUGCUUUCUACUUUAUG |
| | (miR-142-5p binding site) |
| 114 | GACAGUGCAGUCACCCAUAAAGUAGAAAGCACUACUAACAGCACUGGAGGGU |
| | GUAGUGUUUCCUACUUUAUGGAUGAGUGUACUGUG |
| | (miR-142) |
| 185 | GGGAAAUAAGAGAGAAAAGAAGAGUAAGAAGAAAUAUAAGAGCCACC |
| | (5′ UTR) |
| 165 | GGGAAAUAAGAGUCCAUAAAGUAGGAAACACUACAAGAAAAGAAGAGUAAGA |
| | AGAAAUAUAAGAGCCACC |
| | (5′ UTR with miR142-3p binding site at position p1) |
| 166 | GGGAAAUAAGAGAGAAAAGAAGAGUAAUCCAUAAAGUAGGAAACACUACAGA |
| | AGAAAUAUAAGAGCCACC |
| | (5′ UTR with miR 142-3p binding site at position p2) |
| 167 | GGGAAAUAAGAGAGAAAAGAAGAGUAAGAAGAAAUAUAAUCCAUAAAGUAGG |
| | AAACACUACAGAGCCACC |
| | (5′ UTR with miR142-3p binding site at position p3) |
| 168 | ACCCCUAUCACAAUUAGCAUUAA |
| | (miR 155-5p binding site) |
| 169 | UGAUAAUAG [symbol] GCUGGAGCCUCGGUGGCCAUGC |
| | UUCUUGCCCCUUGGGCC [symbol] UCCCCCCAGCCCCU |
| | CUCCCCUUCCUGCACCCGUACCCCC [symbol] GUGGUC |
| | UUUGAAUAAAGUCUGAGUGGGCGGC |
| | (3′ UTR with 3 miR 142-5p binding sites) |
| 170 | UGAUAAUAGGCUGGAGCCUCGGUGGCCAUGCUUCUUGCCCCUUCCAUAAAGU |
| | AGGAAACACUACAUGGGCCUCCCCCCAGCCCCUCCUCCCCUUCCUGCACCCG |
| | UACCCCCGUGGUCUUUGAAUAAAGUCUGAGUGGGCGGC |
| | (3′UTR including miR142-3p binding site) |
| 171 | UGAUAAUAGGCUGGAGCCUCGGUGGCCAUGCUUCUUGCCCCUUGGGCCUCCC |
| | CCCAGUCCAUAAAGUAGGAAACACUACACCCCUCCUCCCCUUCCUGCACCCG |
| | UACCCCCGUGGUCUUUGAAUAAAGUCUGAGUGGGCGGC |
| | (3′UTR including miR142-3p binding site) |
| 172 | UGAUAAUAGGCUGGAGCCUCGGUGGCCAUGCUUCUUGCCCCUUGGGCCUCCC |
| | CCCAGCCCCUCCUCCCCUUCUCCAUAAAGUAGGAAACACUACACUGCACCCG |
| | UACCCCCGUGGUCUUUGAAUAAAGUCUGAGUGGGCGGC |
| | (3′UTR including miR142-3p binding site) |
| 173 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCGUGGUCUUUGAAUAAA |
| | GUUCCAUAAAGUAGGAAACACUACACUGAGUGGGCGGC |
| | (3′UTR including miR142-3p binding site) |
| 174 | UGAUAAUAGUCCAUAAAGUAGGAAACACUACAGCUGGAGCCUCGGUGGCCUA |
| | GCUUCUUGCCCCUUGGGCCUCCCCCCAGCCCCUCCUCCCCUUCCUGCACCCG |
| | UACCCCC [symbol] GUGGUCUUUGAAUAAAGUCUGAG |
| | UGGGCGGC |
| | (3′ UTR with miR 142-3p and miR 126-3p binding sites variant 2) |
| 175 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCCGUGGUCUUUGAAUAAA |
| | GUCUGAGUGGGCGGC |
| | (3′ UTR, no miR binding sites variant 2) |
| 186 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCCUCCAUAAAGUAGGAAA |
| | CACUACAGUGGUCUUUGAAUAAAGUCUGAGUGGGCGGC |
| | (3′ UTR with miR 142-3p binding site variant 3) |
| 177 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 126-3p binding site variant 3) |
| 178 | UGAUAAUAGUCCAUAAAGUAGGAAACACUACAGCUGGAGCCUCGGUGGCCUA |
| | GCUUCUUGCCCCUUGGGCCUCCAUAAAGUAGGAAACACUACAUCCCCCCAGC |
| | CCCUCCUCCCCUUCCUGCACCCGUACCCCCUCCAUAAAGUAGGAAACACUAC |
| | AGUGGUCUUUGAAUAAAGUCUGAGUGGGCGGC |
| | (3′ UTR with 3 miR 142-3p binding sites variant 2) |
| 179 | UGAUAAUAGUCCAUAAAGUAGGAAACACUACAGCUGGAGCCUCGGUGGCCUA |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′UTR with miR 142-3p binding site, P1 insertion variant 2) |
| 180 | UGAUAAUAGGCUGGAGCCUCGGUGGCUCCAUAAAGUAGGAAACACUACACUA |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′UTR with miR 142-3p binding site, P2 insertion variant 2) |
| 181 | UGAUAAUAGGCUGGAGCCUCGGUGGCCUAGCUUCUUGCCCCUUGGGCCUCCA |
| | UAAAGUAGGAAACACUACAUCCCCCCAGCCCCUCCUCCCCUUCCUGCACCCG |
| | UACCCCCGUGGUCUUUGAAUAAAGUCUGAGUGGGCGGC |
| | (3′UTR with miR 142-3p binding site, P3 insertion variant 2) |
| 182 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCCACCCCUAUCACAAUUA |
| | GCAUUAAGUGGUCUUUGAAUAAAGUCUGAGUGGGCGGC |
| | (3′UTR with miR 155-5p binding site variant 2) |
| 183 | UGAUAAUAGACCCCUAUCACAAUUAGCAUUAAGCUGGAGCCUCGGUGGCCUA |
| | GCUUCUUGCCCCUUGGGCCACCCCUAUCACAAUUAGCAUUAAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 3 miR 155-5p binding sites variant 2) |
| 184 | UGAUAAUAGACCCCUAUCACAAUUAGCAUUAAGCUGGAGCCUCGGUGGCCUA |
| | GCUUCUUGCCCCUUGGGCCUCCAUAAAGUAGGAAACACUACAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′UTR with 2 miR 155-5p binding sites and 1 miR 142-3p binding site |
| | variant 2) |

Stop codon = bold
miR 142-3p binding site = underline
miR 126-3p binding site = bold underline
miR 155-5p binding site = italicized
miR 142-5p binding site = italicized and bold underline

In one embodiment, the polynucleotide of the invention comprises a 5′ UTR, a codon optimized open reading frame encoding a polypeptide of interest, a 3′ UTR comprising the at least one miRNA binding site for a miR expressed in immune cells, and a 3′ tailing region of linked nucleosides. In various embodiments, the 3′ UTR comprises 1-4, at least two, one, two, three or four miRNA binding sites for miRs expressed in immune cells, preferably abundantly or preferentially expressed in immune cells.

In one embodiment, the at least one miRNA expressed in immune cells is a miR-142-3p microRNA binding site. In one embodiment, the miR-142-3p microRNA binding site comprises the sequence shown in SEQ ID NO: 116. In one embodiment, the 3′ UTR of the mRNA comprising the miR-142-3p microRNA binding site comprises the sequence shown in SEQ ID NO: 134.

In one embodiment, the at least one miRNA expressed in immune cells is a miR-126 microRNA binding site. In one embodiment, the miR-126 binding site is a miR-126-3p binding site. In one embodiment, the miR-126-3p microRNA binding site comprises the sequence shown in SEQ ID NO: 121. In one embodiment, the 3′ UTR of the mRNA of the invention comprising the miR-126-3p microRNA binding site comprises the sequence shown in SEQ ID NO: 149.

Non-limiting exemplary sequences for miRs to which a microRNA binding site(s) of the disclosure can bind include the following: miR-142-3p (SEQ ID NO: 115), miR-142-5p (SEQ ID NO: 117), miR-146-3p (SEQ ID NO: 135), miR-146-5p (SEQ ID NO: 136), miR-155-3p (SEQ ID NO: 137), miR-155-5p (SEQ ID NO: 138), miR-126-3p (SEQ ID NO: 120), miR-126-5p (SEQ ID NO: 122), miR-16-3p (SEQ ID NO: 139), miR-16-5p (SEQ ID NO: 140), miR-21-3p (SEQ ID NO: 141), miR-21-5p (SEQ ID NO: 142), miR-223-3p (SEQ ID NO: 143), miR-223-5p (SEQ ID NO: 144), miR-24-3p (SEQ ID NO: 145), miR-24-5p (SEQ ID NO: 146), miR-27-3p (SEQ ID NO: 147) and miR-27-5p (SEQ ID NO: 148). Other suitable miR sequences expressed in immune cells (e.g., abundantly or preferentially expressed in immune cells) are known and available in the art, for example at the University of Manchester's microRNA database, miRBase. Sites that bind any of the aforementioned miRs can be designed based on Watson-Crick complementarity to the miR, typically 100% complementarity to the miR, and inserted into an mRNA construct of the disclosure as described herein.

In another embodiment, a polynucleotide of the present invention (e.g., and mRNA, e.g., the 3′ UTR thereof) can comprise at least one miRNA binding site to thereby reduce or inhibit accelerated blood clearance, for example by reducing or inhibiting production of IgMs, e.g., against PEG, by B cells and/or reducing or inhibiting proliferation and/or activation of pDCs, and can comprise at least one miRNA binding site for modulating tissue expression of an encoded protein of interest.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′ UTR to enhance microRNA mediated gene regulation.

At least one miRNA binding site can be engineered into the 3′ UTR of a polynucleotide of the invention. In this context, at least two, at least three, at least four, at least five, at least six, at least seven, at least eight, at least nine, at least ten, or more miRNA binding sites can be engineered into a 3′ UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., myeloid cells, endothelial cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′ UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′ UTR. In another non-limiting example, a miRNA binding site can be engineered near the 5′ terminus of the 3′ UTR and near the 3′ terminus of the 3′ UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In some embodiments, the expression of a polynucleotide of the invention can be controlled by incorporating at least one sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising a ionizable lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment the miRNA sequence in the 5′ UTR can be used to stabilize a polynucleotide of the invention described herein.

In another embodiment, a miRNA sequence in the 5′ UTR of a polynucleotide of the invention can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation to decrease the accessibility to the site of translation initiation. The site of translation initiation can be prior to, after or within the miRNA sequence. As a non-limiting example, the site of translation initiation can be located within a miRNA sequence such as a seed sequence or binding site.

In some embodiments, a polynucleotide of the invention can include at least one miRNA to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA to dampen expression of the encoded polypeptide in a tissue or cell of interest. As a non-limiting example, a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include miR-142-5p, miR-142-3p, miR-146a-5p, and miR-146-3p.

In one embodiment, a polynucleotide of the invention comprises at least one miRNA sequence in a region of the polynucleotide that can interact with a RNA binding protein. In some embodiments, the polynucleotide of the invention (e.g., a RNA, e.g., an mRNA) comprising (i) a sequence-optimized nucleotide sequence (e.g., an ORF) encoding a GMCSF polypeptide (e.g., the wild-type sequence, functional fragment, or variant thereof) and (ii) a miRNA binding site (e.g., a miRNA binding site that binds to miR-142) and/or a miRNA binding site that binds to miR-126.

### IVT Polynucleotide Architecture

In some embodiments, the polynucleotide of the present disclosure comprising an mRNA encoding a GM-CSF polypeptide is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flanking region. This first region can include, but is not limited to, a GM-CSF polypeptide. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a GM-CSF polypeptide can comprise at its 5′ terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of GM-CSF polypeptide or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding the GM-CSF polypeptide. In some embodiments, the UTR is heterologous to the ORF encoding the GM-CSF polypeptide. In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively. Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:87), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide. In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a 0-F1-ATPase, a creatine kinase, a myoglobin, a granulocyte-colony stimulating factor (G-CSF); a collagen (e.g., collagen type I, alpha 2 (CollA2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1). In some embodiments, the 5′ UTR is selected from the group consisting of a β-globin 5′ UTR; a 5′UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase ((3-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′ UTR and/or a 3′ UTR selected from any of the UTRs disclosed herein. In some embodiments, the 5′ UTR comprises:

5′ UTR-001 (Upstream UTR)
(SEQ ID NO: 185)
(GGGAAAUAAGAGAGAAAAGAAGAGUAAGAAGAAAUAUAAGAGCCACC);
5′ UTR-002 (Upstream UTR)
(SEQ ID NO: 89)
(GGGAGAUCAGAGAGAAAAGAAGAGUAAGAAGAAAUAUAAGAGCCACC);
5′ UTR-003 (Upstream UTR) (See WO2016/100812);
5′ UTR-004 (Upstream UTR)
(SEQ ID NO: 90)
(GGGAGACAAGCUUGGCAUUCCGGUACUGUUGGUAAAGCCACC);
5′ UTR-005 (Upstream UTR)
(SEQ ID NO: 91)
(GGGAGAUCAGAGAGAAAAGAAGAGUAAGAAGAAAUAUAAGAGCCACC);
5′ UTR-006 (Upstream UTR) (See WO2016/100812);
5′ UTR-007 (Upstream UTR)
(SEQ ID NO: 92)
(GGGAGACAAGCUUGGCAUUCCGGUACUGUUGGUAAAGCCACC);
5′ UTR-008 (Upstream UTR)
(SEQ ID NO: 93)
(GGGAAUUAACAGAGAAAAGAAGAGUAAGAAGAAAUAUAAGAGCCACC);
5′ UTR-009 (Upstream UTR)
(SEQ ID NO: 94)
(GGGAAAUUAGACAGAAAAGAAGAGUAAGAAGAAAUAUAAGAGCCACC);
5′ UTR-010, Upstream
(SEQ ID NO: 95)
(GGGAAAUAAGAGAGUAAAGAACAGUAAGAAGAAAUAUAAGAGCCACC);
5′ UTR-011 (Upstream UTR)
(SEQ ID NO: 96)
(GGGAAAAAAGAGAGAAAAGAAGACUAAGAAGAAAUAUAAGAGCCACC);
5′ UTR-012 (Upstream UTR)
(SEQ ID NO: 97)
(GGGAAAUAAGAGAGAAAAGAAGAGUAAGAAGAUAUAUAAGAGCCACC);
5′ UTR-013 (Upstream UTR)
(SEQ ID NO: 98)
(GGGAAAUAAGAGACAAAACAAGAGUAAGAAGAAAUAUAAGAGCCACC);
5′ UTR-014 (Upstream UTR)
(SEQ ID NO: 99)
(GGGAAAUUAGAGAGUAAAGAACAGUAAGUAGAAUUAAAAGAGCCACC);
5′ UTR-015 (Upstream UTR)
(SEQ ID NO: 100)
(GGGAAAUAAGAGAGAAUAGAAGAGUAAGAAGAAAUAUAAGAGCCACC);
5′ UTR-016 (Upstream UTR)
(SEQ ID NO: 101)
(GGGAAAUAAGAGAGAAAAGAAGAGUAAGAAGAAAAUUAAGAGCCACC);
5′ UTR-017 (Upstream UTR);
(SEQ ID NO: 102)
(GGGAAAUAAGAGAGAAAAGAAGAGUAAGAAGAAAUUUAAGAGCCACC);
or
5′ UTR-018 (Upstream UTR) 5′ UTR
(SEQ ID NO: 88)
(UCAAGCUUUUGGACCCUCGUACAGAAGCUAAUACGACUCACUAUAGGGAA
AUAAGAGAGAAAAGAAGAGUAAGAAGAAAUAUAAGAGCCACC).

In some embodiments, the 3′ UTR comprises:

142-3p 3′ UTR (UTR including miR 142-3p binding site)
(SEQ ID NO: 104)
(UGAUAAUAGUCCAUAAAGUAGGAAACACUACAGCUGGAGCCUCGGUGGCCA
UGCUUCUUGCCCCUUGGGCCUCCCCCCAGCCCCUCCUCCCCUUCCUGCACCC
GUACCCCCGUGGUCUUUGAAUAAAGUCUGAGUGGGCGGC);
142-3p 3′ UTR (UTR including miR 142-3p binding site)
(SEQ ID NO: 105)
(UGAUAAUAGGCUGGAGCCUCGGUGGCUCCAUAAAGUAGGAAACACUACACA
UGCUUCUUGCCCCUUGGGCCUCCCCCCAGCCCCUCCUCCCCUUCCUGCACCC
GUACCCCCGUGGUCUUUGAAUAAAGUCUGAGUGGGCGGC);
or
142-3p 3′ UTR (UTR including miR 142-3p binding site)
(SEQ ID NO: 106)
(UGAUAAUAGGCUGGAGCCUCGGUGGCCAUGCUUCUUGCCCCUUCCAUAAAG
UAGGAAACACUACAUGGGCCUCCCCCCAGCCCCUCCUCCCCUUCCUGCACCC
GUACCCCCGUGGUCUUUGAAUAAAGUCUGAGUGGGCGGC);
142-3p 3′ UTR (UTR including miR 142-3p binding site)
(SEQ ID NO: 107)
(UGAUAAUAGGCUGGAGCCUCGGUGGCCAUGCUUCUUGCCCCUUGGGCCUCC
CCCCAGUCCAUAAAGUAGGAAACACUACACCCCUCCUCCCCUUCCUGCACCC
GUACCCCCGUGGUCUUUGAAUAAAGUCUGAGUGGGCGGC);
142-3p 3′ UTR (UTR including miR 142-3p binding site)
(SEQ ID NO: 108)
(UGAUAAUAGGCUGGAGCCUCGGUGGCCAUGCUUCUUGCCCCUUGGGCCUCC
CCCCAGCCCCUCCUCCCCUUCUCCAUAAAGUAGGAAACACUACACUGCACCC
GUACCCCCGUGGUCUUUGAAUAAAGUCUGAGUGGGCGGC);
142-3p 3′ UTR (UTR including miR 142-3p binding site)
(SEQ ID NO: 109)
(UGAUAAUAGGCUGGAGCCUCGGUGGCCAUGCUUCUUGCCCCUUGGGCCUCC
CCCCAGCCCCUCCUCCCCUUCCUGCACCCGUACCCCCUCCAUAAAGUAGGAA
ACACUACAGUGGUCUUUGAAUAAAGUCUGAGUGGGCGGC)
142-3p 3′ UTR (UTR including miR 142-3p binding site)
(SEQ ID NO: 110)
(UGAUAAUAGGCUGGAGCCUCGGUGGCCAUGCUUCUUGCCCCUUGGGCCUCC
CCCCAGCCCCUCCUCCCCUUCCUGCACCCGUACCCCCGUGGUCUUUGAAUAA
AGUUCCAUAAAGUAGGAAACACUACACUGAGUGGGCGGC);
3′ UTR-018 (See SEQ ID NO: 150);
3′ UTR (miR 142 and miR126 binding sites variant 1)
(SEQ ID NO: 111)
(UGAUAAUAGUCCAUAAAGUAGGAAACACUACAGCUGGAGCCUCGGUGGCCA
UGCUUCUUGCCCCUUGGGCCUCCCCCCAGCCCCUCCUCCCCUUCCUGCACCC
GUACCCCCCGCAUUAUUACUCACGGUACGAGUGGUCUUUGAAUAAAGUCUGA
GUGGGCGGC)
3′ UTR (miR 142 and miR126 binding sites variant 2)
(SEQ ID NO: 112)
(UGAUAAUAGUCCAUAAAGUAGGAAACACUACAGCUGGAGCCUCGGUGGCCU
AGCUUCUUGCCCCUUGGGCCUCCCCCCAGCCCCUCCUCCCCUUCCUGCACCC
GUACCCCCCGCAUUAUUACUCACGGUACGAGUGGUCUUUGAAUAAAGUCUGA
GUGGGCGGC);
or
3′UTR (miR142-3p binding site variant 3)
(SEQ ID NO: 186)
UGAUAAUAGGCUGGAGCCUCGGUGGCCUAGCUUCUUGCCCCUUGGGCCUCCC
CCCAGCCCCUCCUCCCCUUCCUGCACCCGUACCCCCUCCAUAAAGUAGGAAA
CACUACAGUGGUCUUUGAAUAAAGUCUGAGUGGGCGGC.

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a5′ UTR and/or 3′ UTR sequence provided herein. In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NOs: 185, 88-102, or 165-167 and/or 3′ UTR sequences comprises any of SEQ ID NOs:104-112, 150, 151, or 178, and any combination thereof. In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of SEQ ID NO: 185, SEQ ID NO:193, SEQ ID NO:39, or SEQ ID NO:194 and/or 3′ UTR sequences comprises any of SEQ ID NO:150, SEQ ID NO:175, SEQ ID NO:195, SEQ ID NO:196, SEQ ID NO: 186, SEQ ID NO:177, SEQ ID NO:111, or SEQ ID NO:178, and any combination thereof.

In some embodiments, the 5′ UTR comprises an amino acid sequence set forth in Table 4B. In some embodiments, the 3′ UTR comprises an amino acid sequence set forth in Table 4B. In some embodiments, the 5′ UTR comprises an amino acid sequence set forth in Table 4B and the 3′ UTR comprises an amino acid sequence set forth in Table 4B.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

Regions Having a 5′ Cap

The disclosure also includes a polynucleotide that comprises both a 5′ Cap and a polynucleotide of the present invention (e.g., a polynucleotide comprising a nucleotide sequence encoding a GMCSF polypeptide).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a GMCSF polypeptide) incorporate a cap moiety.

In some embodiments, polynucleotides of the present invention (e.g., a polynucleotide comprising a nucleotide sequence encoding a GMCSF polypeptide) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a GMCSF polypeptide) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly A Tails

In some embodiments, the polynucleotides of the present disclosure (e.g., a polynucleotide comprising a nucleotide sequence encoding a GMCSF polypeptide) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:25).

(SEQ ID NO: 25)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:26).

(SEQ ID NO: 25)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

### Start Codon Region

The invention also includes a polynucleotide that comprises both a start codon region and the polynucleotide described herein (e.g., a polynucleotide comprising a nucleotide sequence encoding a GMCSF polypeptide). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The invention also includes a polynucleotide that comprises both a stop codon region and the polynucleotide described herein (e.g., a polynucleotide comprising a nucleotide sequence encoding a GMCSF polypeptide). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

### Methods of Making Polynucleotides for Use in Treatment of Parkinson's Disease

The present disclosure also provides methods for making a polynucleotide disclosed herein or a complement thereof. In some aspects, a polynucleotide (e.g., an mRNA) disclosed herein, and encoding a GM-CSF molecule can be constructed using in vitro transcription.

In other aspects, a polynucleotide (e.g., an mRNA) disclosed herein encoding a GM-CSF molecule can be constructed by chemical synthesis using an oligonucleotide synthesizer. In other aspects, a polynucleotide (e.g., an mRNA) disclosed herein encoding a GM-CSF molecule is made by using a host cell. In certain aspects, a polynucleotide (e.g., an mRNA) disclosed herein encoding a GM-CSF molecule is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding a GM-CSF molecule. The resultant mRNAs can then be examined for their ability to produce protein and/or produce a therapeutic outcome.

Exemplary methods of making a polynucleotide disclosed herein include: in vitro transcription enzymatic synthesis and chemical synthesis which are disclosed in International PCT application WO 2017/201325, filed on 18 May 2017, the entire contents of which are hereby incorporated by reference.

### Purification

In other aspects, a polynucleotide (e.g., an mRNA) disclosed herein encoding a GM-CSF molecule can be purified. Purification of the polynucleotides (e.g., mRNA) encoding a GM-CSF molecule described herein can include, but is not limited to, polynucleotide clean-up, quality assurance and quality control. Clean-up can be performed by methods known in the arts such as, but not limited to, AGENCOURT® beads (Beckman Coulter Genomics, Danvers, MA), poly-T beads, LNATM oligo-T capture probes (Exiqon, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) encoding a GM-CSF molecule of the disclosure removes impurities that can reduce or remove an unwanted immune response, e.g., reducing cytokine activity.

In some embodiments, the polynucleotide (e.g., mRNA) encoding a GM-CSF molecule of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes a GM-CSF molecule disclosed herein increases expression of the a GM-CSF molecule compared to polynucleotides encoding the GM-CSF molecule purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a GM-CSF molecule. In some embodiments, the purified polynucleotide encodes a human GM-CSF molecule.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (W). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1ψ) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions for Use in Treatment of Parkinson's Disease

The present disclosure provides pharmaceutical formulations comprising any of the LNP compositions disclosed herein, e.g., an LNP composition comprising a polynucleotide comprising an mRNA encoding a GM-CSF molecule.

In some embodiments of the disclosure, the polynucleotides are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations

The polynucleotide comprising an mRNA encoding a GM-CSF molecule of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

In some embodiments, the formulations described herein contain at least one LNP, e.g., one LNP comprising one polynucleotide. As a non-limiting example, the formulations contain 1, 2, 3, 4 or 5 LNPs, e.g., each LNP comprising one polynucleotide. In some embodiments, the LNPs (e.g., the mixture comprising 2, 3, 4, or 5 LNPs) comprise the same polynucleotide. In some embodiments, the LNPs (e.g., the mixture comprising 2, 3, 4 or 5 LNPs) comprise different polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

Table of contents for Examples

| | |
|---|---|
| Example 1 | Methods of making compositions for treatment of Parkinson's disease and |
| | methods of testing such compositions |
| Example 2 | Injection of LNP-formulated Gm-csf mRNA increased plasma GM-CSF, |
| | spleen size and cell number, and WBC counts |
| Example 3 | LNP formulated Gm-csf mRNA increased Tregs leading to neuroprotection in |
| | a 1-Methyl-4-Phenyl-1,2,3,6-Tetrahydropyridine (MPTP)-intoxicated model |
| Example 4 | LNP formulated Gm-csf mRNA transformed CD4+ T cells and induced |
| | myeloid cell populations |
| Example 5 | LNP formulated Gm-csf mRNA treatments led to neuroprotection and anti- |
| | inflammatory responses in an Alpha-Synuclein (α-syn) model of Parkinson's |
| | Disease |
| Example 6 | Injection of LNP-formulated MSA-conjugated Gm-csf mRNA increased |
| | plasma GM-CSF, spleen size, and WBC counts |
| Example 7 | LNP formulated MSA-conjugated Gm-csf mRNA increased Tregs leading to |
| | neuroprotection in a 1-Methyl-4-Phenyl-1,2,3,6-Tetrahydropyridine (MPTP)- |
| | intoxicated model |
| Example 8 | Study of LNP formulated RSA-conjugated Gm-csf mRNA treatments in an |
| | Alpha-Synuclein (α-syn) model of Parkinson's Disease |

### Example 1: Methods of Making Compositions for Treatment of Parkinson's Disease and Methods of Testing Such Compositions

mRNA Synthesis and Formulation

Gm-csf mRNA was synthesized in vitro using T7 RNA polymerase-mediated transcription with N1-methylpseudouridine replacing uridine. The linearized DNA template incorporates the 5′ and 3′ untranslated regions (UTRs) and a poly-A tail as previously described (Bahl et al., (2017) Mol Ther 25, 1316-1327). To increase mRNA translation efficiency, the final mRNA is capped. After purification, the desired mRNA concentration was acquired by diluting mRNA in citrate buffer. Control mRNA NTFIX (nontranslated Factor IX) was synthesized by similar methods.

Lipid nanoparticle (LNP) formulations were prepared by modifying a previously described method (Richner et al., (2017) Cell 168, 1114-1125). Briefly, lipids were dissolved in ethanol at molar ratios of 50:10:38.5:1.5 (ionizable lipid:helper lipid:structural lipid:PEG), and the lipid mixture was combined with an acidification buffer of 50 mM citrate buffer (pH 4.0) containing mRNA at a ratio of 2:1 (aqueous:ethanol) using synchronized syringe pumps (Harvard Apparatus). Formulations were diafiltered and concentrated using 20 mM Tris (pH 7.4) with 8% sucrose via Pellicon XL 100 kDa tangential flow membranes (EMD Millipore), passed through a 0.22 m filter, and frozen until use. The structure and composition of the LNP was as previously described (Sabnis et al., (2018) Mol Ther 26, 1509-1519). Formulations were tested for particle size, RNA encapsulation, and endotoxin. All formulations were found to be 80-100 nm in size by dynamic light scattering and with >80% encapsulation and <10 EU/ml endotoxin. In all of the Examples described below, the ionizable lipid used was Compound 25 and the PEG lipid used was PED-DMG.

Animals, mRNA Treatment, and MPTP Intoxication

Animals were housed, maintained and used for experiments following guidelines set forth by the National Institutes of Health Institutional Review Board and approved by the Animal Care and Use Committee of the University of Nebraska Medical Center. For mouse studies, C57BL/6J mice (6-8 weeks old) were obtained from Jackson Laboratories (Stock #000664). After acclimation, mice were injected intramuscularly (i.m.) with a lipid nanoparticle containing Mus musculus Gm-csf mRNA (Gm-csf mRNA). For dose response studies, mice were injected daily for 4 days at doses ranging from 0.00001 mg/kg to 0.1 mg/kg. For neuroprotection experiments, mice were injected with either vehicle (DPBS, 10 ml/kg body weight) or 1-methyl-4-phenyl-1,2,3,6-tetrahydropyridine hydrochloride (MPTP-HCL) reconstituted in phosphate buffered saline (PBS) obtained from Sigma-Aldrich. Mice received 4 subcutaneous injections of MPTP-HCl (16 mg free base/kg), each administered at 2-hour intervals. MPTP safety precautions were followed in accordance with determined safety and handling protocol (Jackson-Lewis & Przedborski, (2007) Nat Protoc 2, 141-151). On days two and seven post MPTP intoxication, mice were sacrificed, and brains were harvested and processed for evaluation of neuroinflammation and neuronal survival, respectively. For rat studies, 7-week old male Sprague-Dawley (SASCO) rats were ordered from Charles River Laboratories. Rats were injected i.m. with a lipid nanoparticle containing Rattus norvegicus Gm-csf mRNA. For naïve rat studies, animals were injected for 4 consecutive days with either 0.01 mg/kg Gm-csf mRNA or 0.1 mg/kg Gm-csf mRNA and sacrificed on day 5. For human alpha-synuclein (α-Syn) overexpression studies, rats were injected for 4 consecutive days immediately following stereotactic injection, followed by injections every other day until sacrifice. On day 28, rats were sacrificed, and spleens and brains were harvested and processed.

### Stereotactic Injection

Sprague-Dawley rats were anesthetized with 2% isoflurane in O₂ and placed in a stereotaxic device (Leica Biosystems Inc., Buffalo Grove, IL) to secure their skulls. Following skull exposure and formation of a 1-2 mm hole, a sterile Hamilton syringe (model 8100, Thermo Fisher) attached to a 26-gauge needle was inserted into the brain. Vectors were delivered via syringe pump. For α-Syn overexpression, AAV2/1-CBA-HuαSyn-IRES-eGFP-WPRE (Standaert-5713) vector (AAV-α-Syn) and control AAV2/1-IRES-eGFP-WPRE (Standaert-5712) vector (AAV-GFP) were obtained from the University of Iowa (Vector Core, Iowa City, IA). In 3 μl of PBS, 3×10⁹ genomic copies of AAV-vectors were delivered to the left hemisphere above the substantia nigra at the following coordinates relative to the bregma: AP, −5.3 mm; ML, −2.0 mm; DV, −7.5 mm DV.

### Perfusions and Immunohistochemistry

Under terminal anesthesia (Fatal Plus, pentobarbital), mice and rats were perfused via cardiac puncture with DPBS followed by 4% paraformaldehyde (PFA) (Sigma-Aldrich) in DPBS. Whole brains taken from animals 7 days post MPTP were harvested after perfusion to assess survival of dopaminergic neuron cell bodies in the substantia nigra (SN) and termini in the striatum. Frozen midbrains were sectioned at 30 m and were immunostained for tyrosine hydroxylase (TH) (anti-TH, 1:2000, EMD Millipore) and counterstained for Nissl substance (Benner et al., (2004) Proc NatlAcad Sci USA 101, 9435-9440). To assess microglial reactivity, brains were harvested 2 days after MPTP (mice) or day 28 dpi (rats) and midbrain sections (30 μm) were immunostained for Mac-1 (anti-CD11b, 1:1000, AbD Serotech) for mice and Iba-1 (1:1000, VWR) for rat. To assess dopaminergic termini, striatal sections (30 μm) were labeled with anti-TH (1:1000, EMD Millipore). To visualize antibody-labeled tissues, sections were incubated in streptavidin-HRP solution (ABC Elite Vector Kit, Vector Laboratories) and color was developed using an H₂O₂ generation system in the presence of diaminobenzidine (DAB) chromogen (Sigma-Aldrich). Estimated neuron and reactive microglial numbers were quantified via unbiased stereological analysis using StereoInvestigator software (MBF Bioscience) as previously described (Benner et al., (2004) Proc Natl Acad Sci USA 101, 9435-9440). Density of dopaminergic neuron termini in the striatum was determined by digital densitometry using Image J software (National Institutes of Health), as previously described (Benner et al., (2004) Proc Natl Acad Sci USA 101, 9435-9440).

### Suppression Assays

For mouse studies, CD4+CD25+ and CD4+CD25− cells were isolated from spleen using EasySep Mouse CD4+CD25+ Regulatory T Cell Isolation Kit II (StemCell) per the manufacturer's instructions. For rat studies, cells were isolated using EasySep Rat CD4+ T Cell Isolation Kit (StemCell). Isolated rat CD4+ cells were stained with anti-CD25 PE (BD Bioscience) for 20 minutes at a concentration of 0.75 μg/ml per 1.5×10⁷ cells. Anti-PE-magnetic beads from EasySep PE Positive Selection Kit II (StemCell) were then added for the positive magnetic separation of CD4+CD25+ T cells. Isolated cell populations were assessed for purity by flow cytometric analysis and were determined to be >90% CD4+CD25+ and >60% CD4+CD25+FOXP3+ as determined by the expression of intracellular FOXP3. The CD4+CD25− T cell fraction was collected from the untreated groups in both rats and mice and served as the Tresponder (Tresp) population for the suppression assay. Isolated Tresps were labeled with carboxyfluorescein succinimidyl ester (CFSE) (Thermo Fisher). Tregs were serially diluted by 2-fold into wells of a 96-well U bottom microtiter plate and CFSE-stained Tresps were plated at a concentration of 50×10⁶ cells/ml to yield Treg:Tresp ratios of 2:1, 1:1, 1:0.5, 1:0.25, and 1:0.125. Mouse cells were stimulated for proliferation using Dynabeads T-activator CD3/CD28 beads (ThermoFisher) at a 1:1 bead:cell ratio. For rat cell stimulation, beads were conjugated in our laboratory. Dynabeads M-450 epoxy (ThermoFisher) were conjugated using anti-rat CD3 and anti-rat CD28 according to the manufacturer's protocol. The resulting CD3:CD28 ratio was 1:1 and the resulting bead:antibody ratio was 1000 beads:200 μg (100 μg of each antibody). After conjugation, beads were stored at 4° C. at a concentration of 4×10⁷ beads/ml in PBS, pH 7.4 with 0.1% bovine serum albumin (BSA). Stimulated Tresps alone and unstimulated Tresps were plated as controls. Suppression assay cultures were incubated at 37° C. in 5% CO₂ for 3 days, fixed, and analyzed on a BD LSRII flow cytometer. The extent of proliferation by CFSE fluorescence was assessed using FACSDiva Software (BD Biosciences, San Jose, CA).

### Adoptive Transfer

From donor mice treated with Gm-csf mRNA, splenic CD4+CD25+ cells were isolated using the same kits as described in the suppression assay. Recipient mice were intoxicated with MPTP as described and 1×10⁶ CD4+CD25+ cells were adoptively transferred via tail vein injection between 8 and 12 hours post-MPTP treatment. On day seven following administration of MPTP, mice were sacrificed, and brains were harvested and processed.

### Flow Cytometric Assessments

After 4 days of Gm-csf mRNA or protein treatment, whole blood and spleens of rats and mice were collected to determine T cell and B cell profiles via flow cytometric analysis. Whole blood (50 μl) and splenocytes (1×10⁶) were fluorescently labeled using antibodies against extracellular markers for CD3, CD4, CD25, CD8, and B220 and the intracellular marker for FOXP3. Mouse blood and splenocytes were labeled with PerCP-Cy5.5-anti-CD3 (eBioscience), PE-Cy7-anti-CD4 (eBioscience), PE-anti-CD25 (eBioscience) and FITC-anti-CD8 (eBioscience). Rat blood was stained with BV-421-anti-CD3 (BD Bioscience), PerCP-eFluor710-anti-CD4 (eBioscience), PE-anti-CD25 (BD Bioscience), BV-786-anti-CD8 (BD Bioscience) and BUV-737-anti-B220 (BD Bioscience). For intracellular staining of both rat and mouse cells, cells were permeabilized for 45 min at 4° C. using FOXP3/Transcription Factor Staining Buffer Set (eBioscience). Cells were then labeled with APC-anti-FOXP3 (eBioscience) followed by fixation. Samples were processed on a BD LSRII flow cytometer and analyzed using FACSDiva Software (BD Biosciences, San Jose, CA). Cell frequencies were determined from the total lymphocyte population.

### Blood Chemistry and Peripheral Blood Assessments

At the time of sacrifice, 250 μl whole blood was collected into K₂EDTA blood collection tubes for complete blood count (CBC) levels or into heparinized blood collection tubes for blood chemistry and metabolite levels. Following isolation, heparinized blood was centrifuged and plasma was collected. Complete metabolic panels were carried out using VetScan Chemistry Comprehensive Test cartridges (Abaxis) on a VetScan VS2 machine. For CBC analysis, whole blood collected from K₂EDTA tubes was immediately assayed on a VetScan HM5 machine.

### GMCSF Protein Quantification

Prior to treatment, peripheral blood from mice was collected via maxillary bleed. Mice were then treated with Gm-csf mRNA, and after 6 hours, mice were bled again. Plasma was collected by centrifuging whole blood at 10,000 RPM for 10 minutes. After 4 days of Gm-csf mRNA treatment, mice were sacrificed and organs were harvested including spleen, liver, brain, and inguinal lymph nodes. Organs were flash frozen on dry ice. After freezing, 5 mg of tissue was lysed using NP40 Cell Lysis Buffer (Invitrogen), Complete EDTA-free Protease Inhibitor Cocktail (Sigma Aldrich), and PMSF in DMSO. Samples were sonicated, centrifuged, and aliquoted. Using tissue lysate, a Pierce BCA Protein Assay (ThermoFisher) was performed to determine total protein concentration. Following isolation of plasma and tissue, GM-CSF protein levels were determined using Mouse GM-CSF Quantikine ELISA kit (R&D Systems) using the manufacturer's protocol.

### Cytokine Assessments

Before α-syn overexpression and Gm-csf mRNA treatment, rats were bled via maxillary bleed, and peripheral blood was collected as a baseline. After collection, blood was centrifuged at 10,000 RPM for 10 minutes, and plasma was collected and stored at −80° C. After 28 days, at the time of sacrifice, blood and plasma were collected again. After collection, levels of cytokines within plasma before and after treatment were assessed using Rat Cytokine Array Panel A (R&D Systems) according to the manufacturer's protocol.

### RNA Isolation and Transcriptomics

After 4 days of Gm-csf mRNA administration, mice were sacrificed, spleens were harvested, and CD4+ T cells were isolated using EasySep Mouse CD4+ T Cell Isolation Kit (StemCell) per the manufacturer's instructions. Isolated cell purity was assessed via flow cytometric analysis and was determined to be >88% for all isolations. Following cell isolation, total RNA was isolated using RNeasy Mini Kit (Qiagen) under Rnase-free conditions. cDNA was generated from isolated RNA using RevertAid First Strand cDNA Synthesis kit (Thermo Scientific), and preamplification was performed using primer mixes for RT2 PCR array for Mouse T Helper Cell Differentiation (Qiagen). Quantitative RT-PCR was performed on an Eppendorf Mastercycler Realplex EP (Eppendorf). Data analysis was completed using RT2 Profiler PCR Array web-based data analysis software, version 3.5 (Qiagen) and Ingenuity Pathway Analysis (IPA; Qiagen). Only genes that were found to be dysregulated at least 2-fold were invested, as per the requirement of IPA.

### Statistical Analyses

For all studies, data were analyzed using GraphPad Prism 7.0 software (La Jolla, CA). All values are expressed as mean±SEM. Differences in between-group means were analyzed using one-way ANOVA followed by Tukey or Newman-Keuls post hoc test, depending on assay. Significant differences for all studies was selected at p levels <0.05. Measurement of Treg function and dose-dependency were assessed by linear regression analyses as either a function of Treg:Tresp ratio or Gm-csfmRNA dose. Differences in Treg suppressive function were determined by differences between groups in slope or intercept. Slopes for all lines were determined to be significantly non-zero.

### Example 2: Injection of LNP-Formulated Gm-Csf mRNA Increased Plasma GM-CSF, Spleen Size and Cell Number, and WBC Counts

This Example describes the effects of administering LNP (comprising Compound 25) formulated Gm-csf mRNA (Mm.GMCSF construct comprising the sequence shown in Table 4A) to mice. Gm-csfmRNA was synthesized, formulated into lipid nanoparticles (LNP), and administered to 6-8-week-old C57BL/6 mice as described in Example 1.

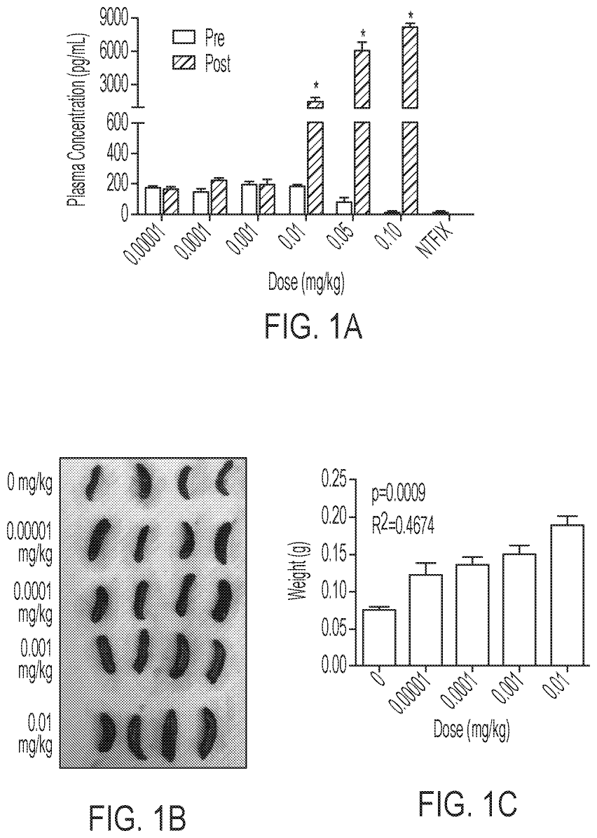

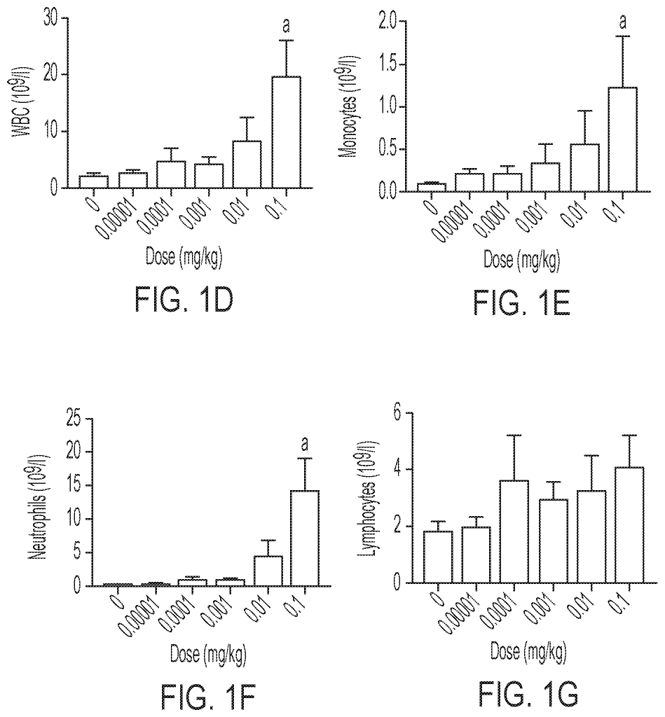

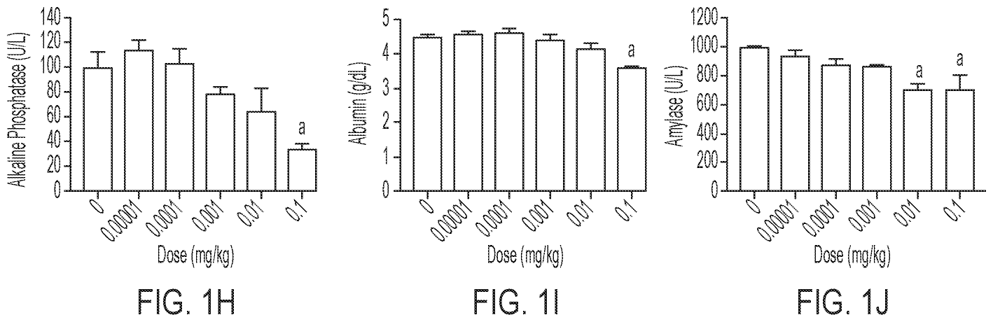

Plasma GM-CSF protein levels were quantified as described in Example 1 in peripheral blood before (pre) and 6 hours after (post) treatment with multiple ascending doses of Gm-csf mRNA or a non-specific mRNA control (NTFIX) (FIG. 1A). As shown in FIG. 1A, Gm-csf mRNA induces detectable GM-CSF protein in plasma within six hours after treatment with 0.01, 0.05, and 0.1 mg/kg. Treatment with 0.01 mg/kg Gm-csf mRNA increased levels from 188 pg/ml to 1487 pg/ml, treatment with 0.05 mg/kg increased levels from 73 pg/ml to 6215 pg/ml, and treatment with 0.1 mg/kg increased levels from 12 pg/ml to 8211 pg/ml. However, elevated GM-CSF protein levels were not detected in plasma of mice treated with lower doses of Gm-csf mRNA or those treated with a non-translatable mRNA control (NTFIX). Also, no increase in GM-CSF protein was detected in spleen, liver, brain, or inguinal lymph nodes of mice treated with LNP formulated Gm-csf mRNA as all tissue levels remained below 71 pg/ml for all treatment groups. Tissues isolated from untreated animals ranged from 10-50 pg/ml GM-CSF protein and were not different from LNP formulated Gm-csf mRNA treatment.

Spleens of mice treated with multiple ascending doses of LNP formulated Gm-csfmRNA were imaged (FIG. 1B). Spleen weight was quantified four days after initial treatment, and linear regression analysis of organ weight was performed. Treatment with ascending doses of LNP formulated Gm-csf mRNA resulted in splenomegaly that was found to be dose-dependent by linear regression analysis (R²=0.46, P=0.0009) (FIG. 1B and FIG. 1C).

Absolute counts of white blood cells (WBC), monocytes, neutrophils, and lymphocytes within whole blood following treatment was determined. Along with increased spleen size, parallel increases in peripheral white blood cells (WBC) were observed (FIGS. 1D-1G). Absolute blood cell counts revealed increases in WBC (FIG. 1D), monocytes (FIG. 1E) and neutrophils (FIG. 1F) that paralleled increased dosing of LNP formulated Gm-csfmRNA, but were only increased with 0.1 mg/kg doses. Lymphocyte populations were only slightly affected (FIG. 1G).

Complete metabolic panels of isolated whole blood from treated mice were carried out as described in Example 1. Blood chemistry profiles for alkaline phosphatase, albumin and amylase following treatment are shown in FIG. 1H, FIG. 1I, and FIG. 1J, respectively. Overall, comprehensive metabolic panels were not altered by treatment as levels of creatinine, total bilirubin, alanine aminotransferase, glucose, urea nitrogen, sodium, calcium, globulin, and phosphorus were unchanged by treatment, whereas alkaline phosphatase (FIG. 1H), albumin (FIG. 1I), and amylase (FIG. 1J) decreased in 0.01 and/or 0.1 mg/kg-treated animals.

Taken together, these results demonstrate that treatment of mice with LNP formulated Gm-csf mRNA resulted in elevated GM-CSF protein levels, increased spleen size, and changes in complete blood counts and peripheral blood chemistry profiles.

### Example 3: LNP Formulated Gm-Csf mRNA Increased Tregs Leading to Neuroprotection in a 1-Methyl-4-Phenyl-1,2,3,6-Tetrahydropyridine (MPTP)-Intoxicated Model

This Examples describes the effects of administering LNP (comprising Compound 25) formulated Gm-csf mRNA (Mm.GMCSF construct comprising the sequences shown Table 4A) to C57BL/6 mice or MPTP-intoxicated C57BL/6 mice. Treatment of mice with native recombinant GM-CSF protein was used as a comparator. Synthesis and formulation of LNP formulated Gm-csf mRNA, treatment and MPTP intoxication of mice, flow cytometric assessments of peripheral blood, suppression assays, and perfusions and immunohistochemistry were performed as described in Example 1.

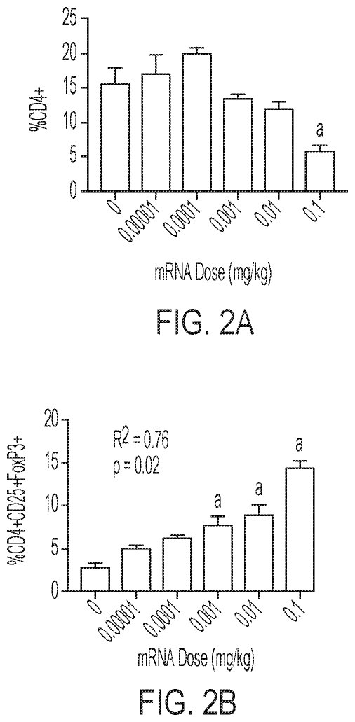

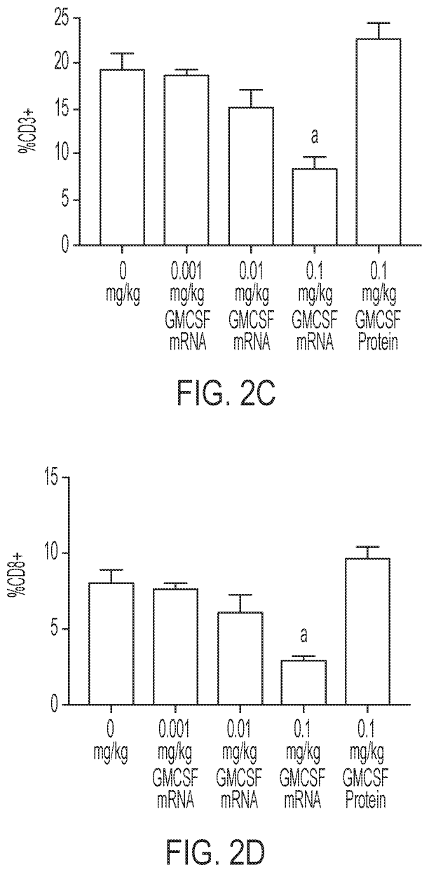

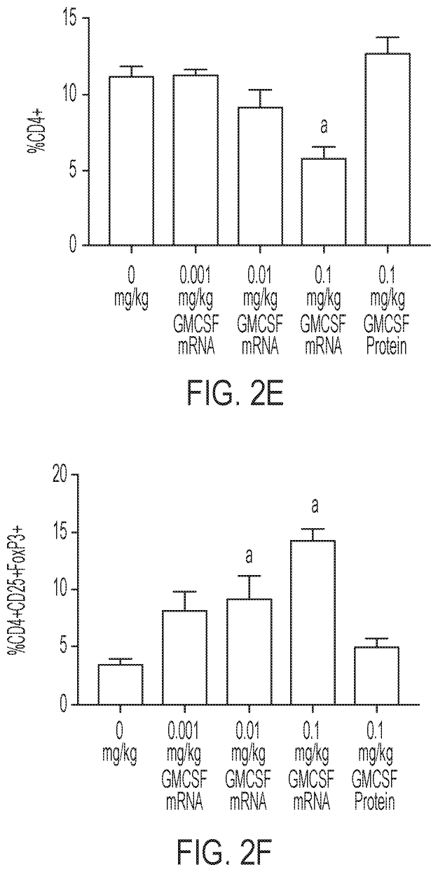

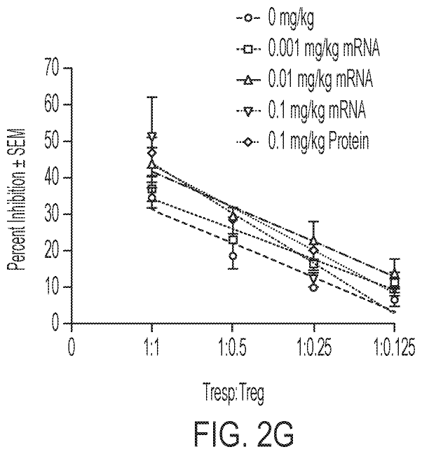

CD4+ T cell and T regulatory cell (Treg) frequency in peripheral blood following treatment of C57BL/6 mice was determined by flow cytometry. Treatment of mice with doses of LNP formulated Gm-csf mRNA ranging from 0 to 0.01 mg/kg showed no change in CD4+ T cell frequencies (FIG. 2A) in peripheral blood. However, 0.1 mg/kg treatment reduced CD4+ T cell frequencies from those observed in untreated animals. In contrast, flow cytometric analysis indicated a dose-dependent increase in CD4+CD25+FOXP3+ regulatory T cell frequencies as revealed by linear regression (R²=0.76, P=0.02) (FIG. 2B). Treg frequencies in peripheral blood increased from 2.8±0.4 to 14.3±0.98% after treatment with escalating doses of Gm-csf mRNA. Similarly, comparing mRNA to protein treatment indicated that administration of 0.1 mg/kg LNP formulated Gm-csfmRNA decreased CD3, CD4, and CD8 frequencies within lymphocyte populations in peripheral blood compared to controls and GM-CSF protein-treated mice (FIG. 2C-2F). Administration of LNP formulated Gm-csf mRNA increased CD4+CD25+FOXP3+ cell frequency from 3.5±0.49 to 14.3±0.99% (FIG. 2F). Treatment at 0.001 and 0.01 mg/kg Gm-csf mRNA or 0.1 mg/kg recombinant GM-CSF protein also increased CD4+CD25+FOXP3+ cell frequencies to 8.2±1.6, 9.2±2.0, and 4.9±0.77%. Overall, these results demonstrate that increasing doses of LNP formulated Gm-csfmRNA resulted in an increase in CD4+CD25+FOXP3+ Treg cells in mice.

Next, the ability of Tregs isolated from treated and untreated animals to inhibit T responder cells (Tresps) was assessed by a suppression assay as described in Example 1. As shown in FIG. 2G, splenic Treg isolated from Gm-csf m RNA treated animals suppressed the proliferation of CD3/CD28-stimulated, carboxyfluorescein succinimidyl ester (CFSE)-stained CD4+CD25−Tresps. See also, Saunders et al., (2012) J Neuroimmune Pharmacol 7, 927-938; Quah & Parish (2010) J Vis Exp. Determination of elevation using linear regression analysis indicated an enhanced suppressive capacity of Tregs isolated from mice treated with LNP formulated Gm-csf mRNA at 0.01 mg/kg (p=0.007) or 0.1 mg/kg (p=0.04) compared to CD4+CD25+ Tregs from non-mRNA-treated controls, but no significant difference in suppressive activity was observed when compared with Tregs from GM-CSF protein-treated animals. Elevations in slope indicated greater inhibitory capacity at lower Tresp:Treg ratios.

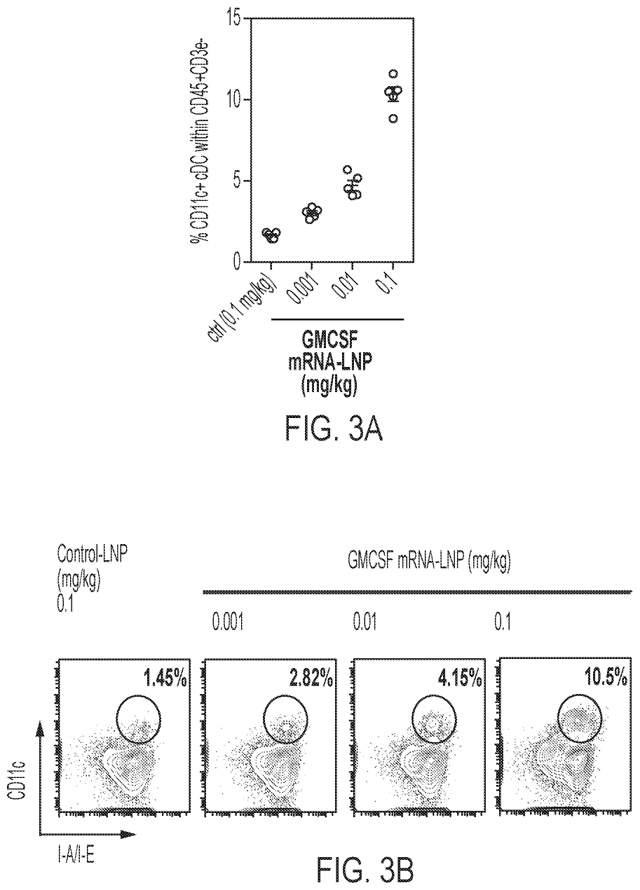

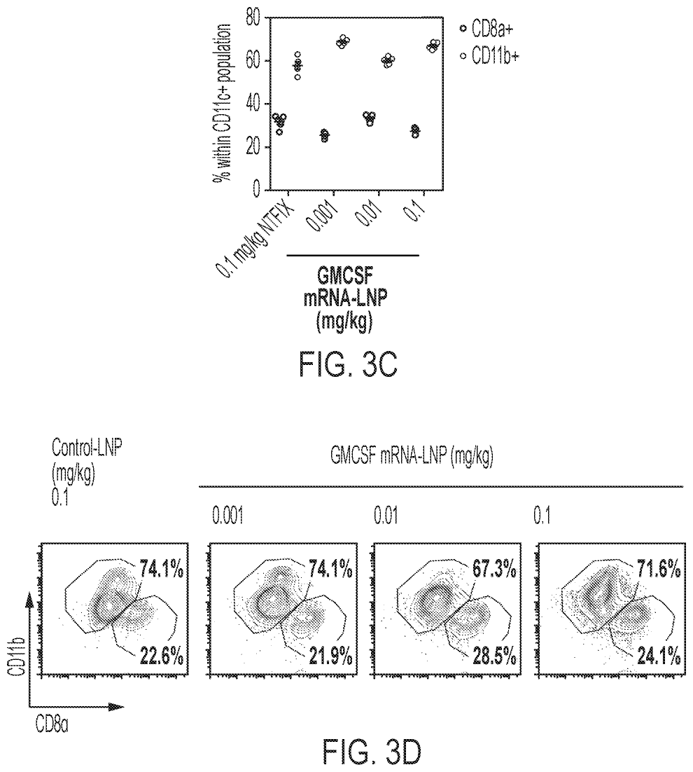

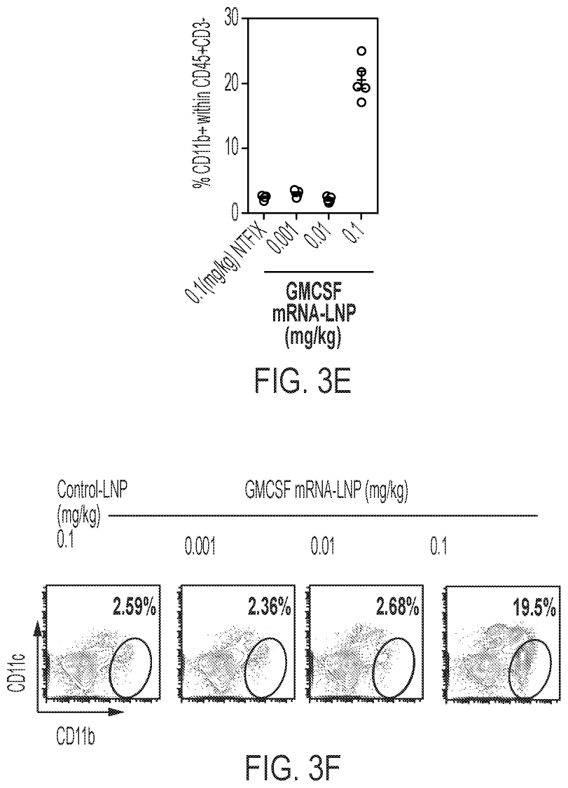

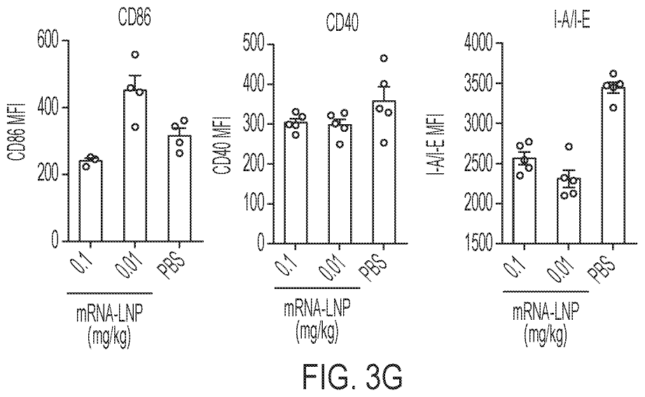

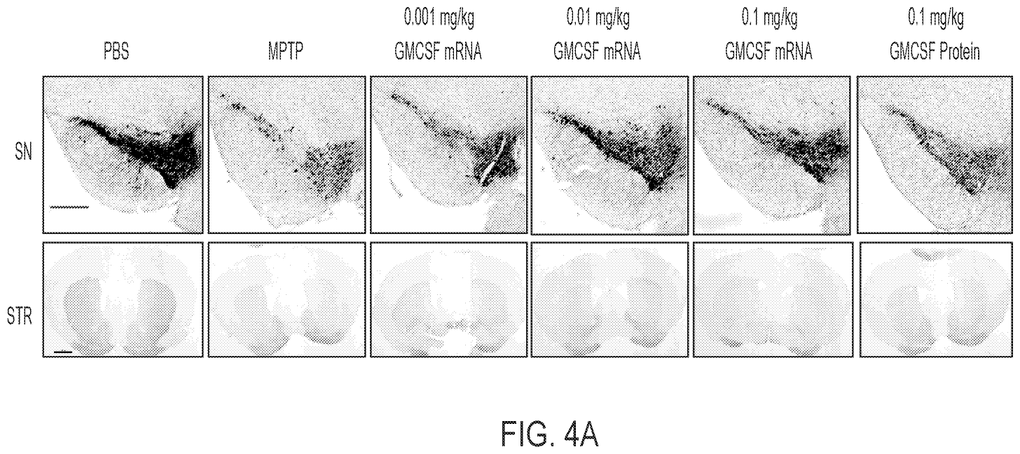

In addition to the % Treg changes observed and described above, increased frequency of splenic CD11c+MHCII+ classical dendritic cells (cDC) was observed in mice treated with LNP formulated Gm-csf mRNA dosed at 0.001-0.1 mg/kg (FIGS. 3A and 3B). CD11b and CD8a expression were used to further characterize the cDC subsets expanded by Gm-csf mRNA. Both subsets expanded to a similar extent when treated with LNP formulated Gm-csf mRNA dosed at 0.001-0.1 mg/kg (FIGS. 3C and 3D). The group treated with 0.1 mg/kg formulated Gm-csf mRNA showed expansion of the CD11b+Cd11c− myeloid population (FIGS. 3E and 3F). To determine whether LNP formulated Gm-csf mRNA altered the maturation status of the expanded CD11c+ dendritic cell population, expression of maturation markers such as CD86, CD40 and Class II (IA-IE) were evaluated by flow cytometry. The mean fluorescence intensity (MFI) of Class II (IA-IE) was reduced upon treatment with 0.01 or 0.1 mg/kg LNP formulated Gm-csf mRNA (FIG. 3G).

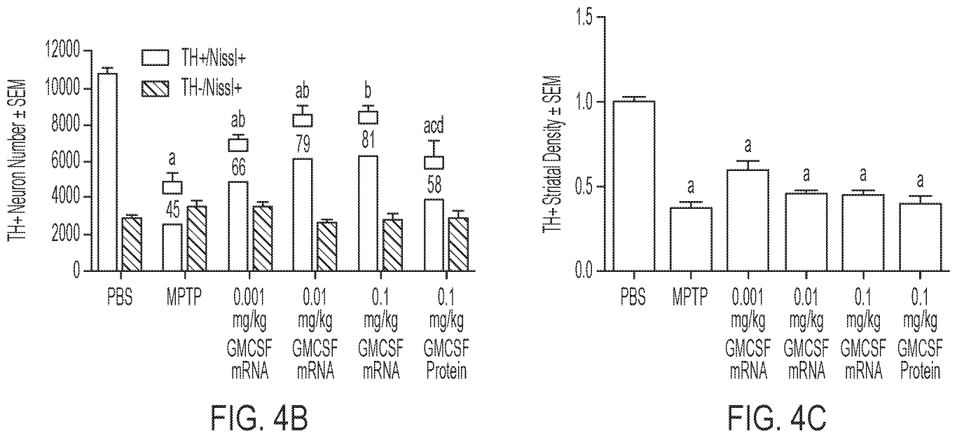

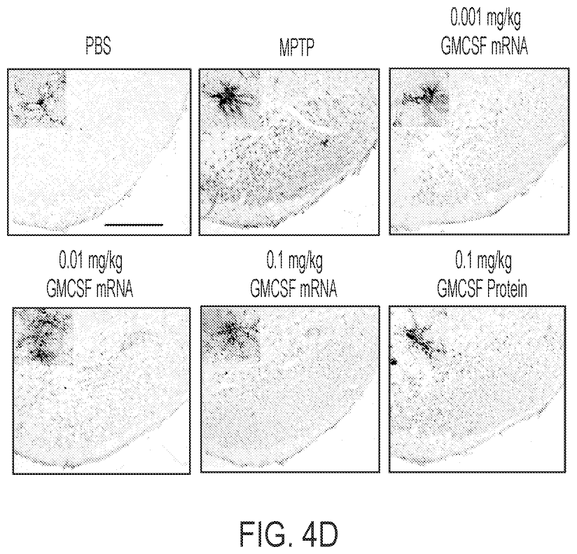

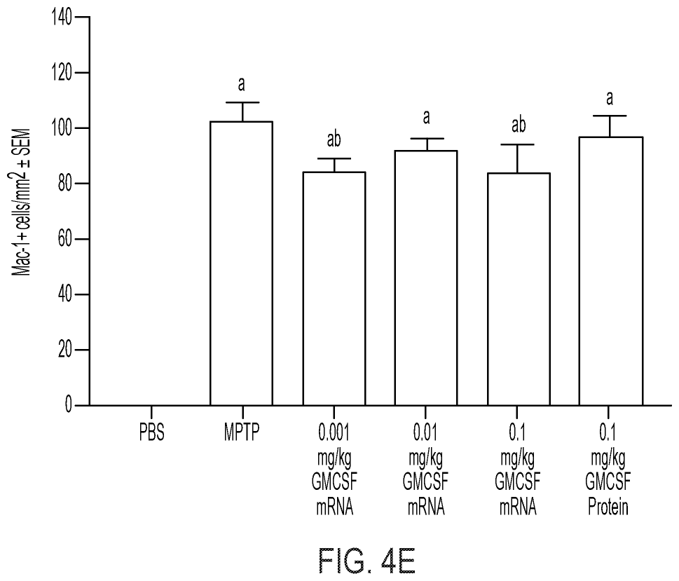

To determine the effect of treatment on neuroprotective activities, mice were pretreated for four days with either GM-CSF protein or LNP formulated Gm-csf mRNA followed by MPTP intoxication. Photomicrographs of immunostained sections of frozen midbrain were evaluated to assess survival of dopaminergic (TH+/Nissl+) neurons within the substantia nigra (SN) and TH+ cell termini within the striatum (STR) of mice. After MPTP intoxication, the total number of surviving nigral dopaminergic (TH+/Nissl+) neurons was assessed along with their striatal projections (FIG. 4A). Numbers of surviving dopaminergic neurons were decreased from 10771 ±356 to 4893±489 when treated with MPTP compared to untreated animals (FIG. 4B). Treatment with GM-CSF protein yielded a 13% (6226±866) increase in neuronal survival when compared to MPTP alone. Treatment with increasing doses of LNP formulated Gm-csf mRNA resulted in a 21% (7162±327), 34% (8576±494), and 36% (8758±291) increases in dopaminergic neuronal survival compared to MPTP alone. All LNP formulated Gm-csf mRNA doses yielded higher TH+ neuronal counts than MPTP intoxication alone. In particular, treatment with 0.1 mg/kg LNP formulated Gm-csf mRNA was not significantly different from non-lesioned controls, supporting the neuroprotective effect of Gm-csf mRNA. Numbers of non-dopaminergic (TH−/Nissl+) neurons remained unchanged regardless of treatment due to their lack of MPTP susceptibility (Otto & Unsicker (1993) J Neurosci Res 34, 382-393; Jackson-Lewis & Przedborski (2007) Nat Protoc 2, 141-151). However, striatal termini were not spared with any treatment, indicating inherent MPTP toxicities (FIG. 4C).

Next, changes in the MPTP-induced inflammatory response were evaluated. Two days after MPTP administration, a time of peak inflammation and neuronal death, brains were harvested to assess reactive microglia as determined by Mac-1 expression and amoeboid morphology (FIG. 4D) (See also., Kurkowska-Jastrzebska et al., (1999) Exp Neurol 156, 50-61). MPTP increased numbers of reactive microglia, elevating counts from 0 cells/mm² for PBS control mice to 104±5 cells/mm² after MPTP intoxication (FIG. 4E). Treatment with ascending doses of LNP formulated Gm-csfmRNA (0.001 mg/kg to 0.1 mg/kg) decreased reactive microglial counts to 85±4, 92±5, and 85±10 cells/mm², respectively. Treatment with 0.001 and 0.1 mg/kg LNP formulated Gm-csf mRNA resulted in a statistically significant attenuation of microglial responses. Treatment with recombinant GM-CSF protein decreased microglial cell counts to 98±7 cells/mm². Overall, these results demonstrate that LNP formulated Gm-csf mRNA treatment resulted in an attenuation of MPTP-induced nigrostriatal neurodegeneration and microglial activation in mice.

### Example 4: LNP Formulated Gm-Csf mRNA Transformed CD4+ T Cells and Induced Myeloid Cell Populations

This Example describes the neuroprotective and anti-inflammatory effects provided by the LNP (comprising Compound 25) formulated Gm-csf mRNA (Mm.GMCSF construct comprising the sequences shown) treatment described in Example 3.

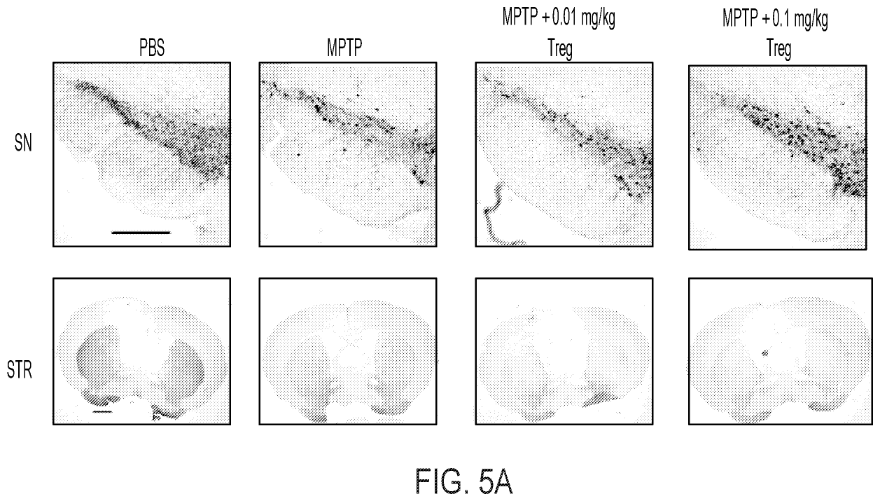

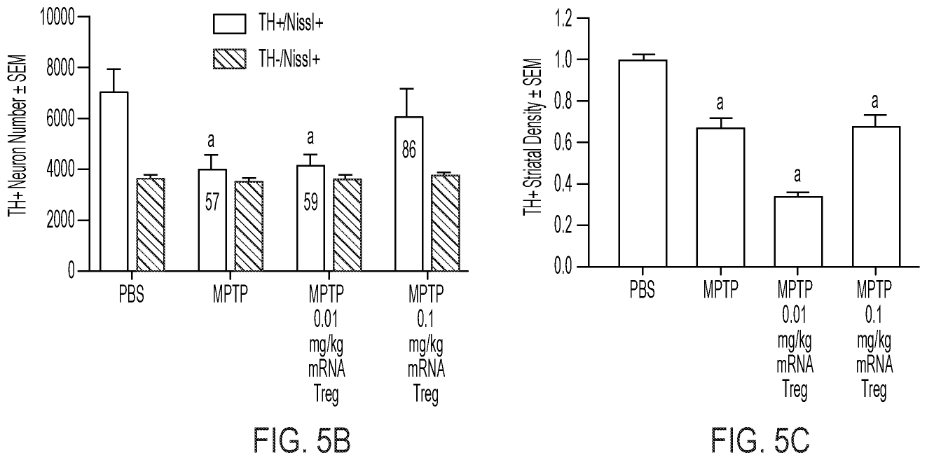

CD4+CD25+ T cells from Gm-csf mRNA-treated donors were isolated and adoptively transferred into MPTP-intoxicated recipient mice as described in Example 1. After seven days, ventral midbrains and striatum were assessed for TH+ dopaminergic neuron survival (FIG. 5A). MPTP intoxication reduced neuron numbers from 7065±878 for PBS controls to 4042±547 (FIG. 5B). Adoptive transfer of CD4+CD25+ cells from mice treated with 0.1 mg/kg LNP formulated Gm-csf mRNA increased TH+/Nissl+ neuron survival 6098±1115 compared to MPTP intoxication alone. CD4+CD25+ cells isolated from 0.01 mg/kg-treated mice did not enhance the level of neuronal survival above the MPTP lesion. Densitometric analysis of striatal termini was not changed after adoptive transfers (FIG. 5C).

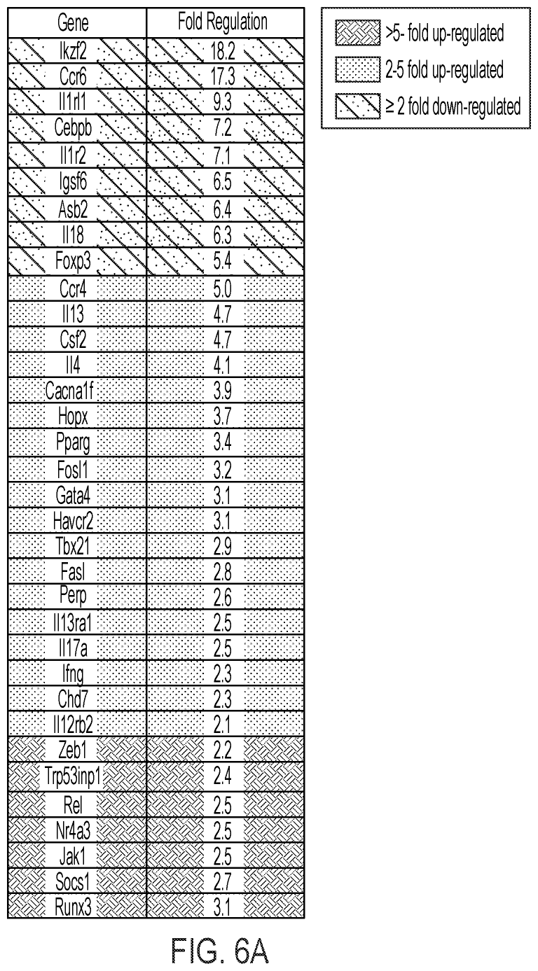

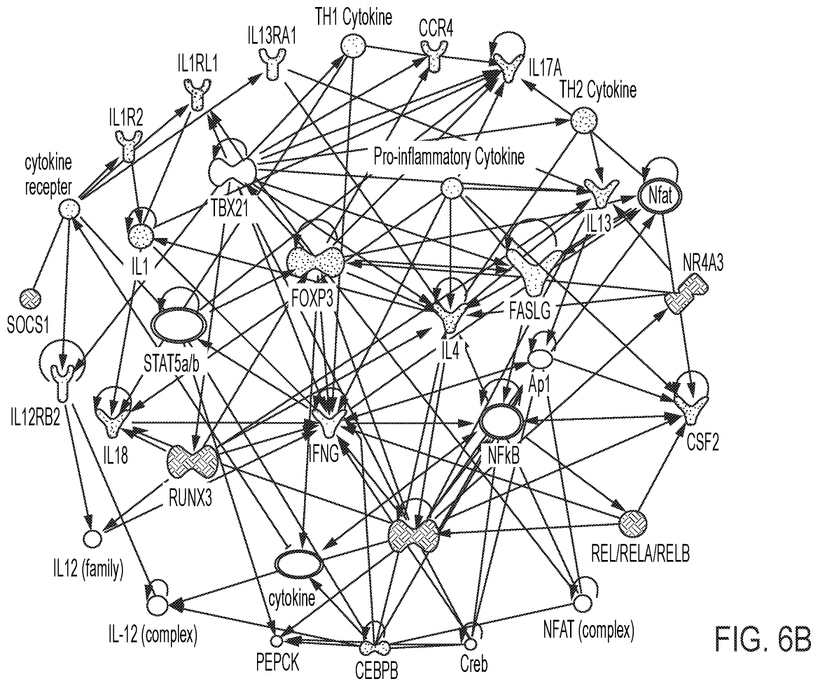

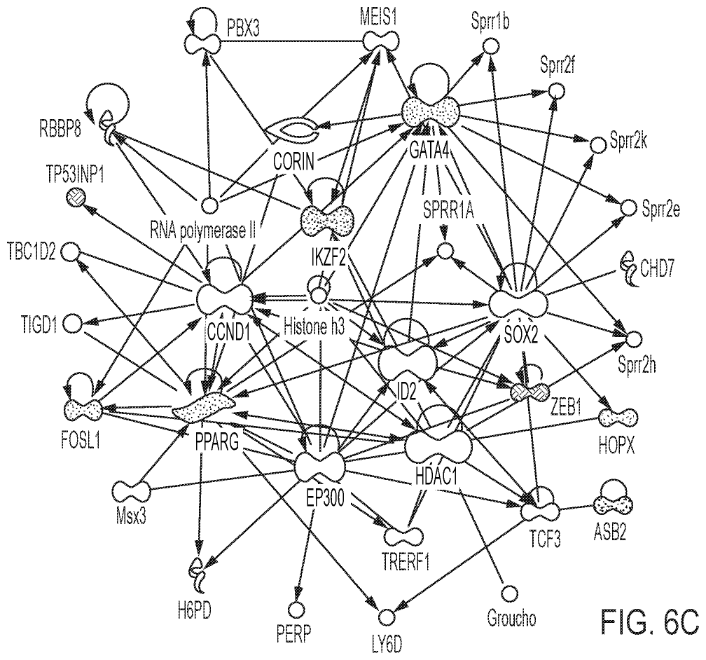

Next, the ability of LNP formulated Gm-csf mRNA to alter the transcriptomic phenotype of the overall CD4+ T cell population was evaluated. To this end, animals were treated with either PBS or 0.1 mg/kg LNP formulated Gm-csfmRNA and the resulting CD4+ T cell population was isolated and analyzed for transcriptomic changes as described in Example 1. LNP formulated Gm-csf mRNA treatment led to both up- and down-regulation of genes associated with various T cell populations (FIGS. 6A-6B). Compared to non-mRNA-treated controls, genes upregulated from 2- to 5-fold included Ccr4, Il13, Csf2, Il4, Cacna1f, Hopx, Pparg, Fosl1, Gata4, Havcr2, Tbx21, Fasl, Perp, Il13ra1, IlI7a, Ifng, Chd7, and Il12rb2. Genes upregulated more than 5-fold included Foxp3, Il18, Asb2, Igsf6, Illr2, Cebpb, Illr11, Ccr6, and Ikzfz, and genes that were downregulated more than 2-fold were Zeb1, Trp53inp1, Rel, Nr4a3, Jak1, Socs1, and Runx3. Network mapping of the resulting gene expression using Ingenuity Pathway Analysis (IPA) indicated the dysregulation of two main networks: Hematological System Development and Function (FIG. 6B); and Cellular and Tissue Development (FIG. 6C). Genetic analysis within these two networks was linked to changes in functional elements associated with T cell differentiation, lymphopoiesis, development, and quantity. This analysis suggests that, in some embodiments, treatment with LNP formulated Gm-csfmRNA positively affects T cell differentiation and lymphocyte number. While both pro- and anti-inflammatory genes were upregulated, many modulations involved genes associated with induction and stabilization of Tregs and Th2 effectors, suggesting, in some embodiments, a potential shift from pro- to anti-inflammatory in the overall T cell phenotype.

### Example 5: LNP Formulated Gm-Csf mRNA Treatments LED to Neuroprotection and Anti-Inflammatory Responses in an Alpha-Synuclein (α-Syn) Model of Parkinson's Disease

This Example describes the neuroprotection and anti-inflammatory responses in an alpha-syn model of Parkinson's Disease.

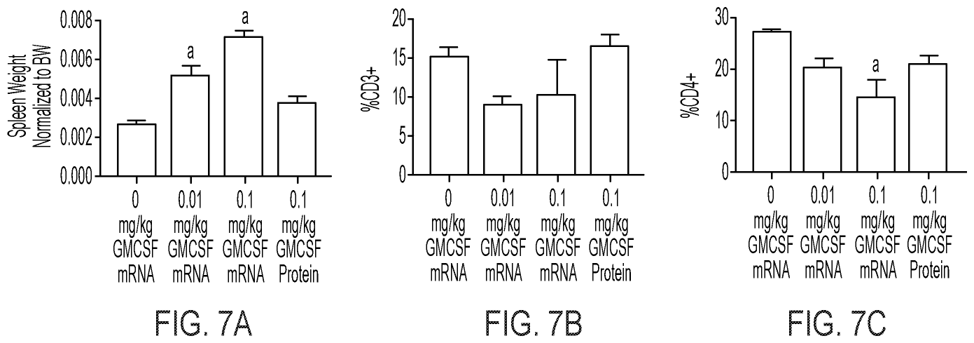

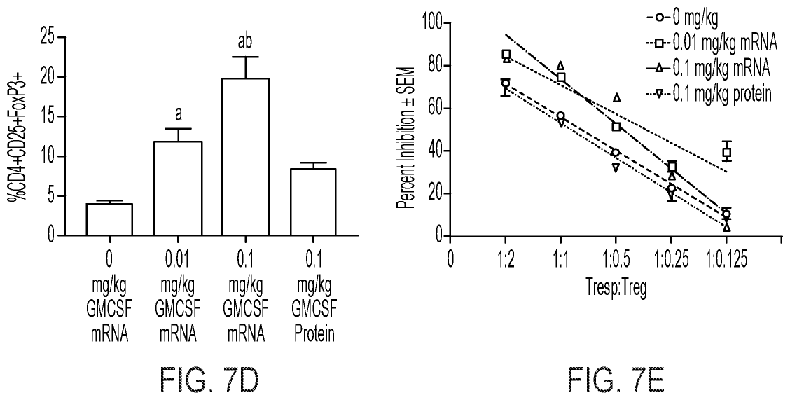

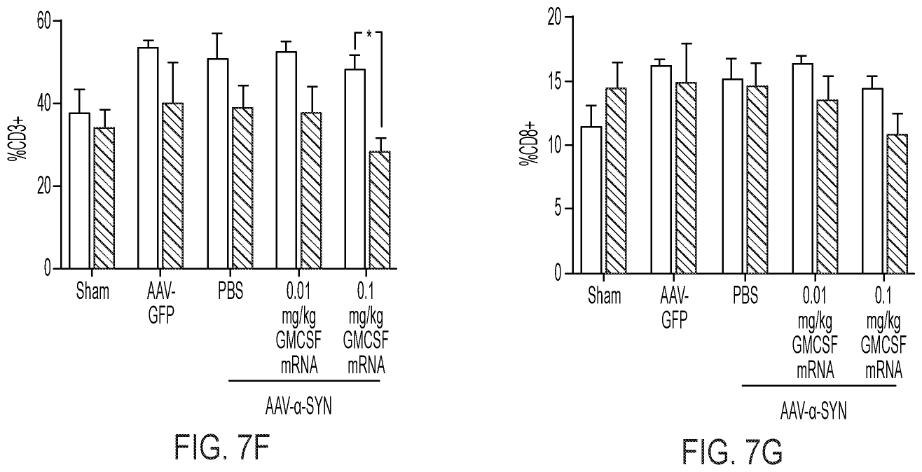

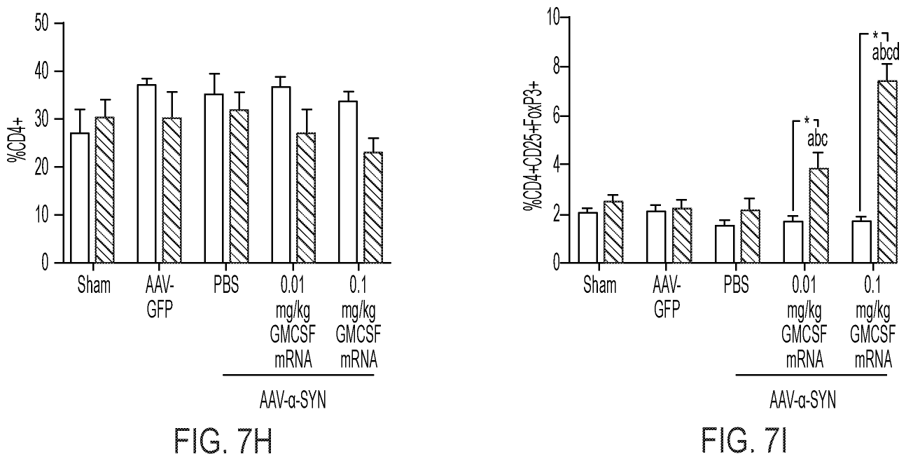

Naïve rats were treated for four days with LNP (comprising Compound 25) formulated rat Gm-csfmRNA (Rn.GMCSF construct comprising the sequences shown in Table 4A) at a dose of 0.01 or 0.1 mg/kg or rat recombinant GM-CSF protein at a dose of 0.1 mg/kg. Both mRNA doses resulted in the same splenomegaly observed in mice (FIG. 7A). Flow cytometric analysis of T cell populations in peripheral blood indicated no change in CD3 percentage with all treatments (FIG. 7B), a decrease in CD4 percentage with 0.1 mg/kg Gm-csf mRNA treatment (FIG. 7C), and an increase in CD4+CD25+FOXP3+ Treg percentages with both mRNA treatments (FIG. 7D). Treg frequencies were increased from 4.1±0.26% in untreated animals to 12±1.5% in 0.01 mg/kg LNP formulated Gm-csf mRNA-treated animals, 20±2.4% in 0.1 mg/kg LNP formulated Gm-csf mRNA-treated animals, and 8.6±0.66% in GM-CSF protein-treated animals, mimicking the observations in mice. CD4+CD25+ Treg cells enriched from each treatment group were then assessed for changes in their suppressive function to inhibit proliferation of CD4+CD25−Tresps (FIG. 7E). Treatment with both dosages of LNP formulated Gm-csf mRNA resulted in enhanced suppressive function compared to either PBS controls or GM-CSF protein-treated CD4+CD25+ Treg (R²≥0.87, P<0.01).

Due to the ability of LNP formulated rat Gm-csf mRNA treatment to selectively increase Treg frequency and function in healthy, naïve animals, the effects of LNP formulated Gm-csf mRNA was next evaluated in a parkinsonian model utilizing human α-syn overexpression. Rats were administered a unilateral stereotactic injection of PBS (Sham), AAV-GFP (Vector control), or AAV-α-syn, followed by LNP formulated Gm-csf mRNA administered intramuscularly the first 4 days and then every other day thereafter. After 28 days, T cell levels were assessed in peripheral blood (FIG. 7F-7I). Levels of CD3+ cells were decreased from pretreatment baseline in animals receiving AAV-α-syn+0.1 mg/kg LNP formulated Gm-csfmRNA and were not significantly altered by other treatments (FIG. 7F). Levels of CD4+ and CD8+ cells were also not significantly affected by any treatment; however, a mild decreasing trend was observed with mRNA treatment (FIGS. 7G and 7H). Recapitulating observations in mice, LNP formulated Gm-csf mRNA treatment significantly increased CD4+CD25+FOXP3+ Treg frequencies within the lymphocyte population of peripheral blood (FIG. 7I). Treatment with 0.01 and 0.1 mg/kg LNP formulated Gm-csf mRNA in α-syn overexpressing animals increased Treg frequencies from 2.5 to 4.0 and 7.4%, respectively.

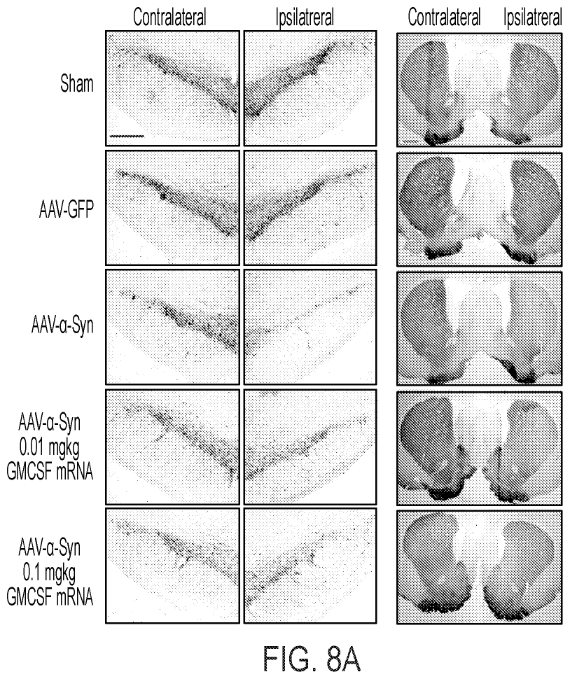

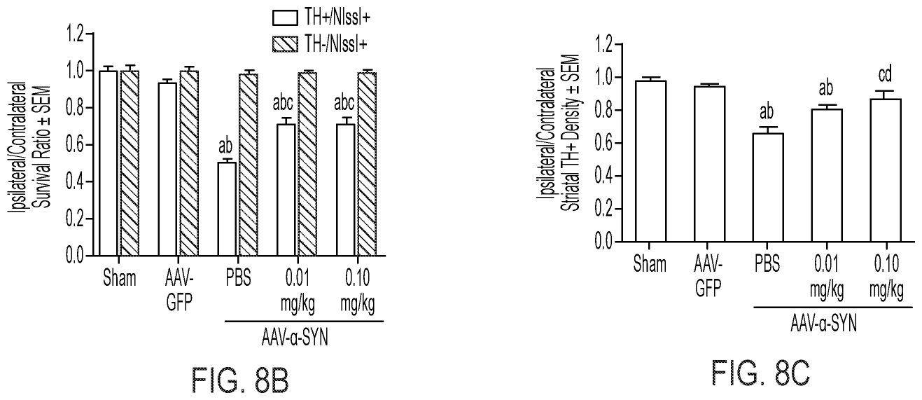

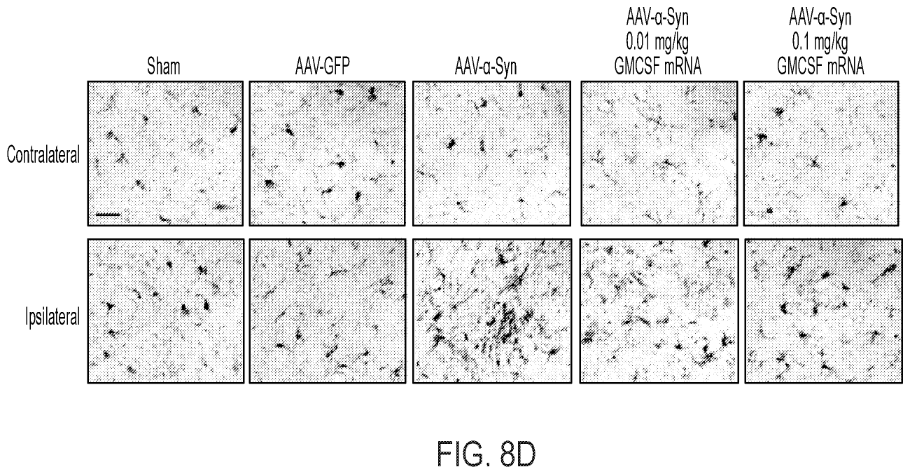

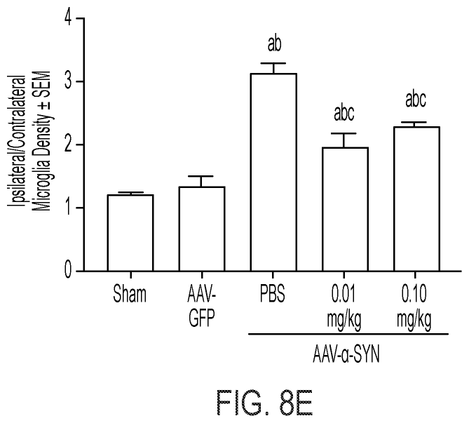

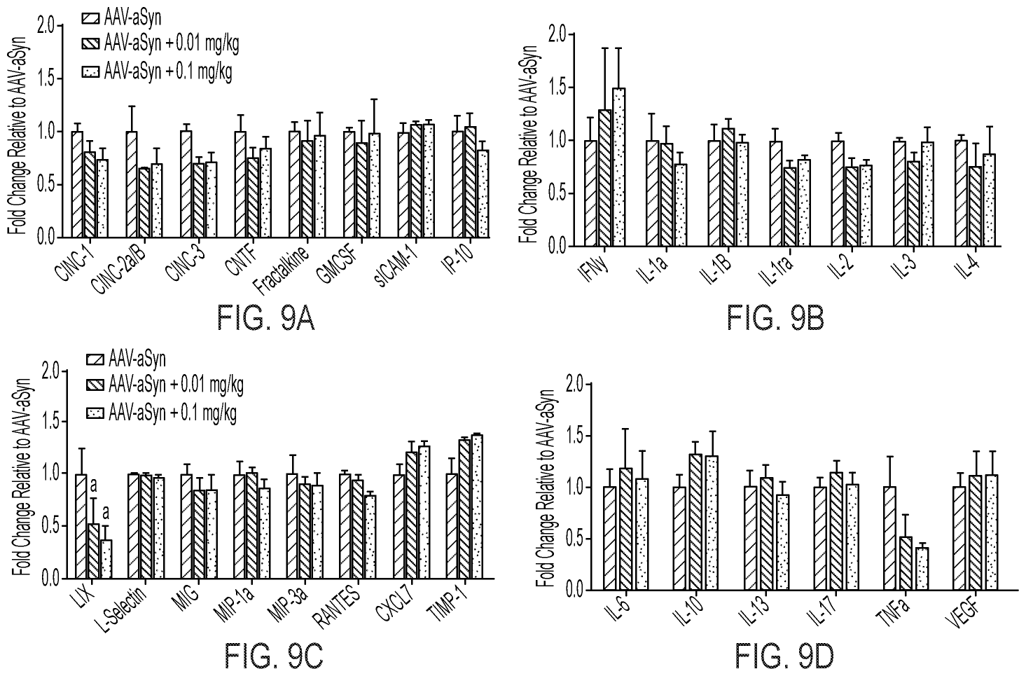

The neuroprotective capacity of LNP formulated Gm-csf mRNA in alpha-syn overexpressing rats was evaluated. AAV-α-syn infection led to a diminution of dopaminergic neurons as determined by loss of TH expression compared to Sham- or AAV-GFP-treated animals (FIG. 8A). Overexpression of α-syn markedly reduced neuron counts by 49.3%. However, treatment with LNP formulated Gm-csf mRNA rescued the neuronal loss by sparing 57% of the neurons (28.2% and 28.8% losses) regardless of dose, thus demonstrating, in some embodiments, a neuroprotective potential (FIG. 8B). Similarly, treatment with 0.01 and 0.1 mg/kg of LNP formulated Gm-csfmRNA also increased dopaminergic termini survival, resulting in a 13.7% and 27.9% level of protection, respectively compared to AAV-α-syn+PBS treatment (FIG. 8C). The levels of reactive microglia as Iba-1+ amoeboid microglia within the substantia nigra was also assessed (FIG. 8D). AAV-vector administration did not elevate reactive microglial ratios over Sham injection, whereas overexpression of α-syn increased reactive microglia by 3-fold (FIG. 8E). Treatment with 0.01 or 0.1 mg/kg of LNP formulated Gm-csf mRNA significantly attenuated the α-syn-associated microglial response with fold ratios diminished to 1.96±0.22 and 2.28±0.09, respectively. Along with microglial responses, cytokine protein levels within plasma were also assessed following 28 days of AAV-α-syn overexpression to determine changes in the inflammatory response associated with disease (FIGS. 9A-9D). Treatment with 0.01 mg/kg LNP formulated Gm-csfmRNA resulted in decreased CINC-1, CINC-2αβ, CINC-3, CNTF, IP-10, LIX, IL-1ra, IL-2, and TNFα (p=0.09). Treatment with 0.1 mg/kg LNP formulated Gm-csf mRNA resulted in decreased CINC-1, CINC-2αβ, CINC-3, CNTF, IP-10, LIX, MIP-1α, RANTES, IL-1α, IL-1ra, IL-2, and TNFα relative to levels observed in AAV-α-syn overexpression alone. Proteins found to be increased in both treatment doses were CXCL7, TIMP-1, IFNγ, and IL-10. Levels of LIX were downregulated from AAV-α-syn overexpression alone (FIG. 9C).

Taken together, these results demonstrate that LNP formulated rat Gm-csf mRNA treatment in an alpha-syn model of Parkinson's disease resulted in neuroprotective activities, enhanced neuronal survival and attenuation of microglial-associated inflammation.

### Example 6: Injection of LNP-Formulated MSA-Conjugated Gm-Csf mRNA Increased Plasma GM-CSF, Spleen Size, and WBC Counts

This Example describes the effects of administering to mice LNP (comprising Compound 25) formulated with Gm-csf mRNA conjugated to mouse albumin (MSA) (MSA-mmGMCSF construct comprising the sequence shown in Table 4A). MSA-conjugated Gm-csfmRNA was synthesized, formulated into lipid nanoparticles (LNP), and administered intramuscularly to mice at a dose of 0.001 mg/kg, 0.01 mg/kg, or 0.1 mg/kg on Day 0. Rm-GM-CSF protein control was administered intraperitoneally to mice once a day for five days on Days −4, −3, −2, −1 and 0. Mice were sacrificed at 1, 3, and 5 days post-treatment for peripheral blood assessments, protein quantification, and immunohistochemistry studies.

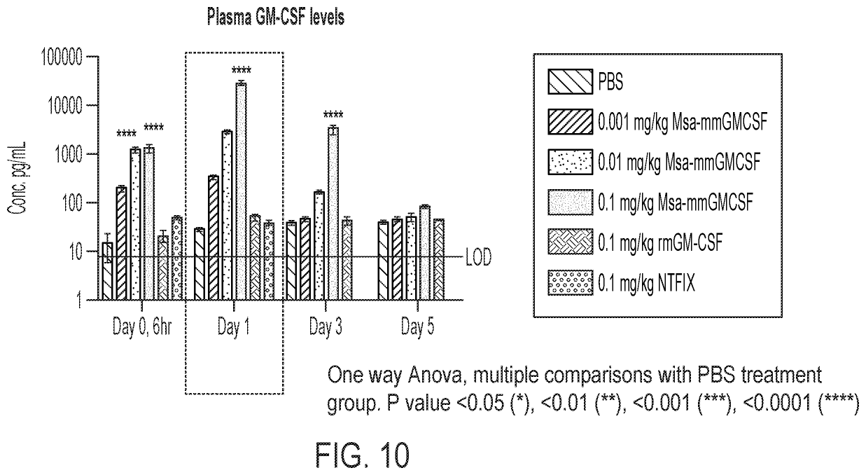

Plasma GM-CSF protein levels were quantified as described in Example 1 in peripheral blood before (pre) and 6 hours, and 1, 3, and 5 days after (post) treatment with multiple ascending doses of MSA-conjugated Gm-csf mRNA, a GM-CSF protein control, or a non-specific mRNA control (NTFIX) (FIG. 10). As shown in FIG. 10, MSA-conjugated Gm-csf mRNA induced detectable GM-CSF protein in plasma within six hours after treatment with 0.001, 0.01, and 0.1 mg/kg. Treatment with MSA-conjugated Gm-csf mRNA at all dosages (0.001, 0.01, and 0.1 mg/kg) resulted in significantly higher levels of plasma GM-CSF protein relative to treatment with 0.1 mg/kg of GM-CSF protein at both 6 hours and 1 day post treatment. Where the dosage of MSA-conjugated Gm-csf mRNA and GM-CSF protein (i.e., 0.1 mg/kg) were administered, the increased protein level in subjects treated with the MSA-conjugated Gm-csf mRNA relative to those treated with GM-CSF protein was observed until the fifth day after dosing. Maximal plasma GM-CSF protein levels were observed at about 1 day post-treatment with 0.001, 0.01, and 0.1 mg/kg doses of MSA-conjugated Gm-csf mRNA.

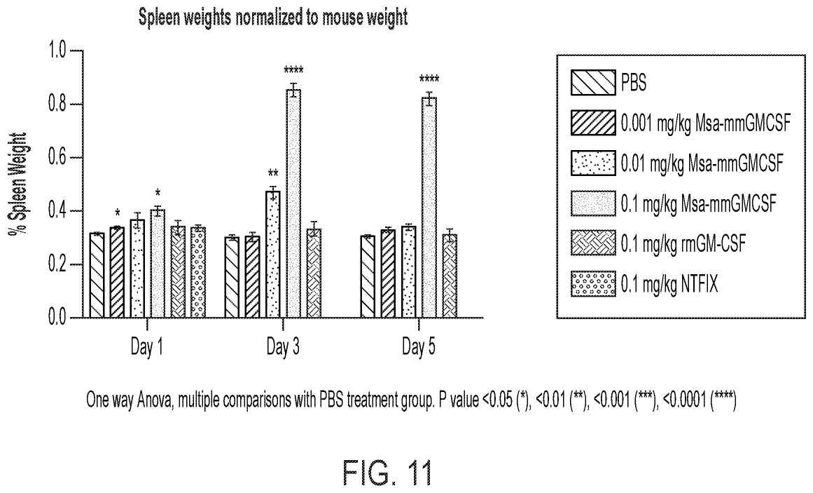

Harvested spleens of mice treated with multiple ascending doses of LNP formulated MSA-conjugated Gm-csf mRNA were measured to determine spleen weight one, three, and five days after initial treatment. The highest dose of 0.1 mg/kg MSA-conjugated Gm-csfmRNA resulted in the largest increase in spleen weight, with maximal splenomegaly occurring about 3 days post-treatment (FIG. 11). The corresponding dosage of GM-CSF protein did not produce a comparable splenomegaly.

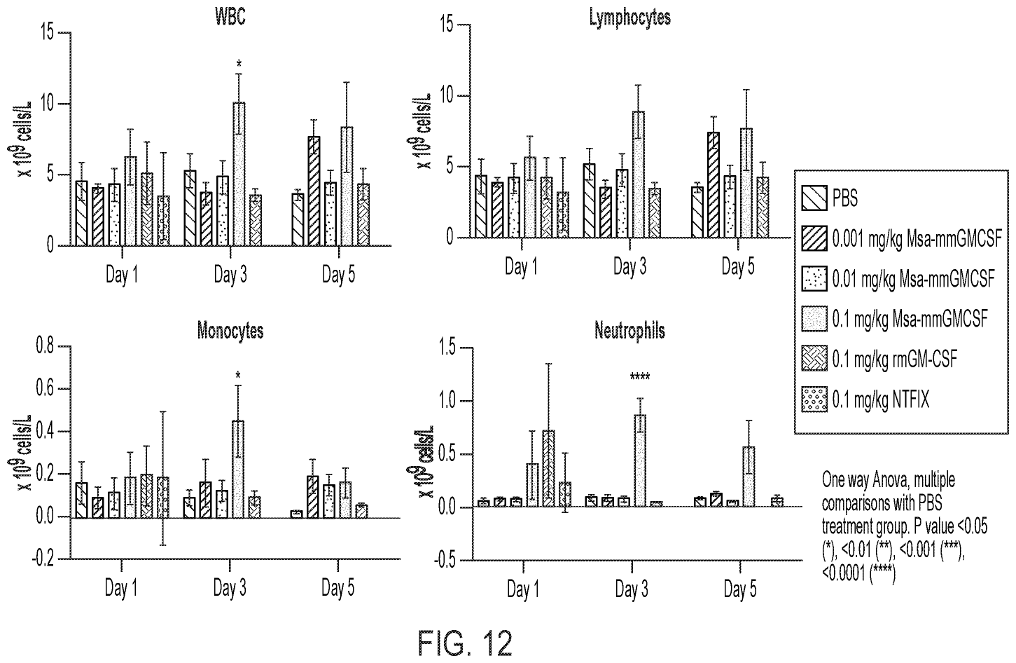

Absolute counts of white blood cells (WBC), monocytes, neutrophils, and lymphocytes within whole blood following treatment was determined. Along with increased spleen size, parallel increases in peripheral white blood cells (WBC) were observed (FIG. 12). Absolute blood cell counts revealed maximal increases in WBC, monocytes, neutrophils and lymphocytes occurred at about 3 days post-treatment.

Taken together, these results demonstrate that treatment of mice with LNP formulated MSA-conjugated Gm-csf mRNA resulted in elevated GM-CSF protein levels, increased spleen size, and changes in complete blood counts.

### Example 7: LNP Formulated MSA-Conjugated Gm-csfmRNA Increased Tregs Leading to Neuroprotection in a 1-Methyl-4-Phenyl-1,2,3,6-Tetrahydropyridine (MPTP)-Intoxicated Model

This Examples describes the effects of administering LNP (comprising Compound 25) formulated Gm-csf mRNA (MSA-mmGMCSF construct comprising the sequence shown in Table 4A) to mice or MPTP-intoxicated mice. Treatment of mice with native recombinant GM-CSF protein was used as a comparator. Treatment and MPTP intoxication of mice, flow cytometric assessments of peripheral blood, suppression assays, and perfusions and immunohistochemistry were performed as described in Example 1.

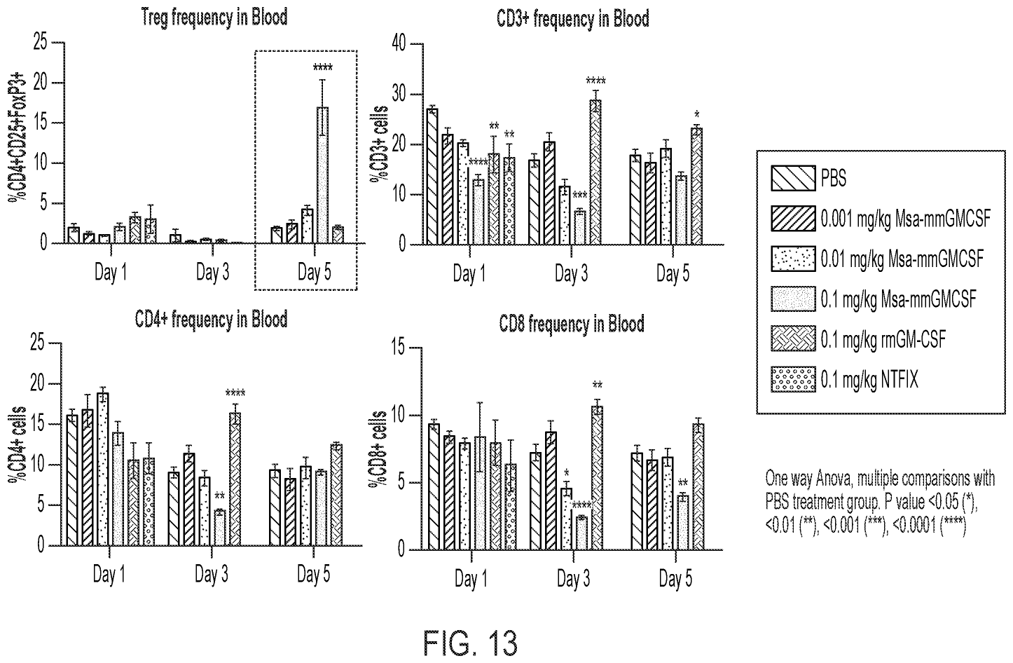

CD4+ T cell and T regulatory cell (Treg) frequency in peripheral blood following treatment of mice was determined by flow cytometry. Flow cytometric analysis indicated a dose-dependent increase in CD4+CD25+FOXP3+ regulatory T cell frequencies following treatment of mice with doses of LNP formulated MSA-conjugated Gm-csfmRNA. A maximal increase in Treg frequency in peripheral blood was observed at about 5 days post-treatment. Similarly, comparing mRNA to protein treatment indicated that administration of 0.1 mg/kg LNP formulated MSA-conjugated Gm-csf mRNA decreased CD3, CD4, and CD8 frequencies within lymphocyte populations in peripheral blood compared to controls and GM-CSF protein-treated mice (FIG. 13). Overall, these results demonstrate that increasing doses of LNP formulated Gm-csf mRNA resulted in an increase in CD4+CD25+FOXP3+ Treg cells in mice, peaking at day 5.

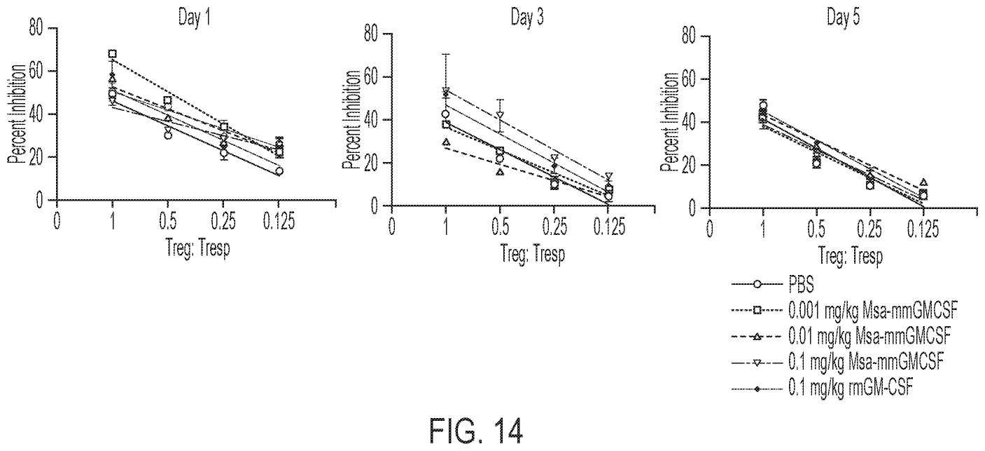

Next, the ability of Tregs isolated from treated and untreated animals to inhibit T responder cells (Tresps) was assessed by a suppression assay as described in Example 1. As shown in FIG. 14, splenic Treg isolated from Gm-csf m RNA treated animals suppressed the proliferation of CD3/CD28-stimulated, carboxyfluorescein succinimidyl ester (CFSE)-stained CD4+CD25−Tresps. Maximal inhibitory capacity of the Tregs occurred at about 3 days post-treatment. The time of maximal Treg activity was characterized as the point of MPTP intoxication.

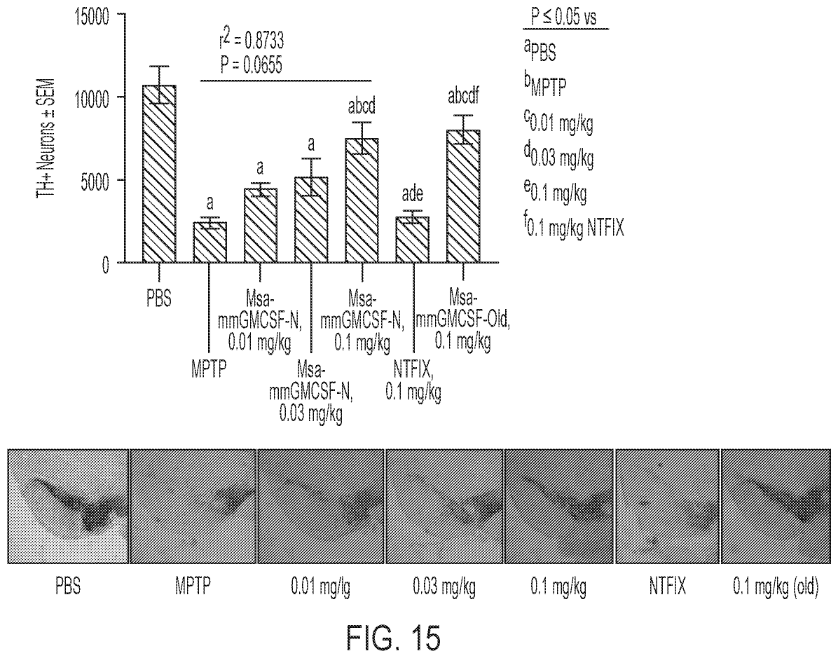

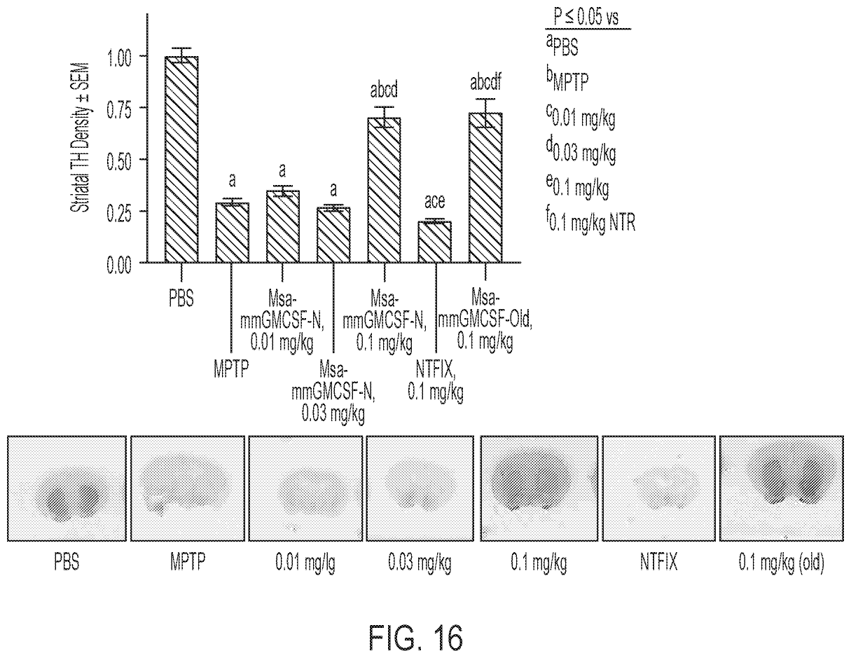

To determine the effect of treatment on neuroprotective activities, mice were pretreated for four days with either GM-CSF protein or LNP formulated Gm-csf mRNA followed by MPTP intoxication. Photomicrographs of immunostained sections of frozen midbrain were evaluated to assess survival of dopaminergic (TH+/Nissl+) neurons within the substantia nigra (SN) and TH+ cell termini within the striatum (STR) of mice. After MPTP intoxication, e.g., after 3 days post-treatment, the total number of surviving nigral dopaminergic (TH+/Nissl+) neurons was assessed along with their striatal projections (FIG. 15). Treatment with MSA-conjugated GM-CSF protein yielded an increase in neuronal survival when compared to MPTP alone or the non-translatable control. Treatment with increasing doses of LNP formulated MSA-conjugated Gm-csf mRNA resulted in corresponding increases in dopaminergic neuronal survival compared to MPTP alone. All LNP formulated MSA-conjugated Gm-csfmRNA doses yielded higher TH+ neuronal counts than MPTP intoxication alone. In particular, treatment with 0.1 mg/kg LNP formulated Gm-csf mRNA was not significantly different from non-lesioned controls, supporting the neuroprotective effect of Gm-csf mRNA. Moreover, the LNP formulated MSA-conjugated Gm-csf mRNA could be stored at 4° C. for at least 6 months without diminishing this effect. While administration of 0.01 or 0.03 mg/kg MSA-conjugated Gm-csf mRNA yielded similar strial TH density values to MPTP alone, administration of the LNP formulated MSA-conjugated Gm-csf mRNA at the highest dosage (0.1 mg/kg) produced a comparable measure of straial TH density to non-lesioned controls, indicating some protective capability at higher dosages (FIG. 16). Moreover, this neuroprotective activity was not observed with administration of the GM-CSF protein alone, even at dosages of 0.1 mg/kg (FIGS. 18 and 19).

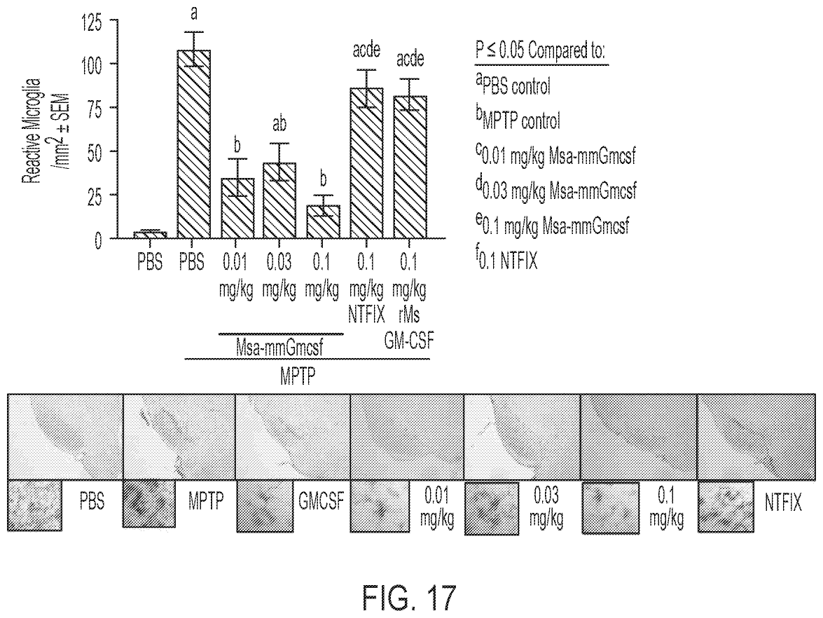

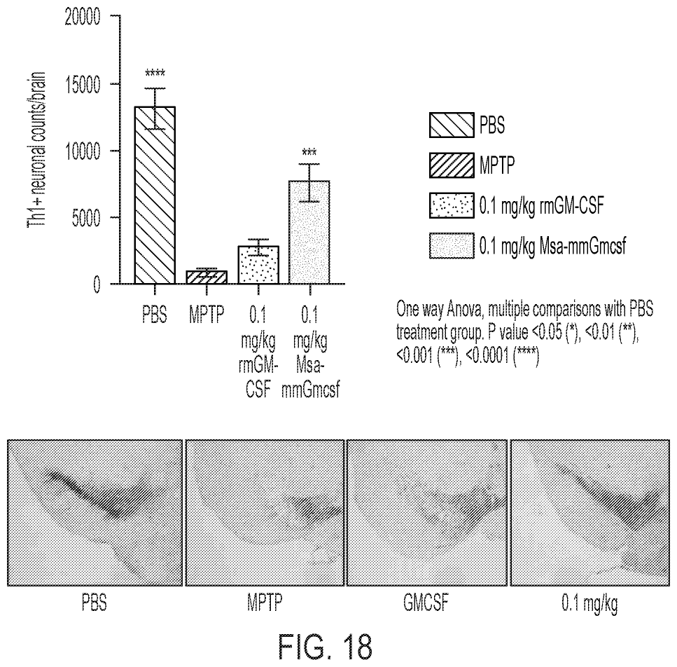

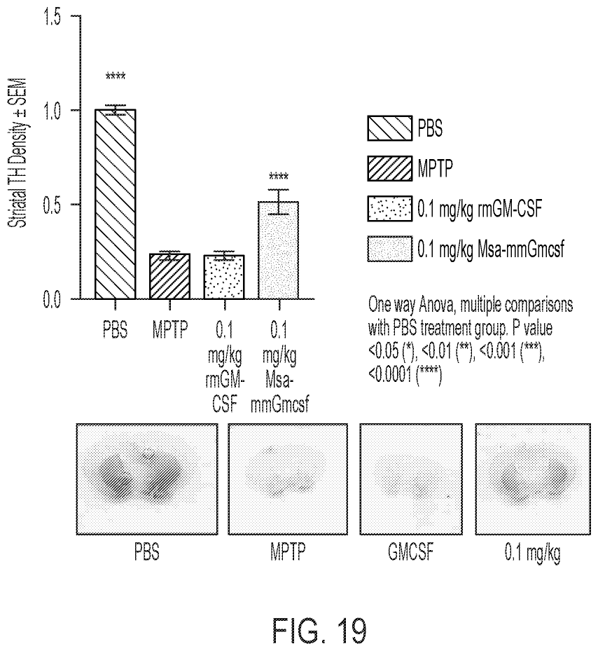

Next, changes in the MPTP-induced inflammatory response were evaluated. Two days after MPTP administration, a time of peak inflammation and neuronal death, brains were harvested to assess reactive microglia as determined by Mac-1 expression and amoeboid morphology (FIG. 17) Treatment with LNP formulated MSA-conjugated Gm-csfmRNA resulted in a statistically significant attenuation of microglial responses, producing a greater reduction than treatment with recombinant GM-CSF protein. Overall, these results demonstrate that LNP formulated MSA-conjugated Gm-csf mRNA treatment resulted in an attenuation of MPTP-induced nigrostriatal neurodegeneration and microglial activation in mice.

### Example 8: Study of LNP Formulated RSA-Conjugated Gm-csfmRNA Treatments in an Alpha-Synuclein (α-syn) Model of Parkinson's Disease

This Example describes the effect of LNP formulated RSA-conjugated Gm-csf in an alpha-syn model of Parkinson's Disease.

Rats were administered a unilateral stereotactic injection of PBS (Sham), AAV-GFP (Vector control), or AAV-α-syn, followed by LNP (comprising Compound 25) formulated rat albumin (RSA)-conjugated Gm-csf mRNA (RSA-rnGMCSF construct comprising the sequences shown in Table 4A) administered intramuscularly at the time of AAV administration and then every seven days thereafter at dosages of 0.1 and 0.3 mg/kg. Control studies were performed dosing a non-translatable Gm-csf mRNA at a dose of 0.3 mg/kg intramuscularly, or rat recombinant GM-CSF protein at a dose of 0.1 mg/kg intraperitoneally. Mice were bled at days 7, 14, 21, and 28 to asses peripheral T cell and B cell populations. Mice were sacrificed on day 28 and spleens and brains were harvested.

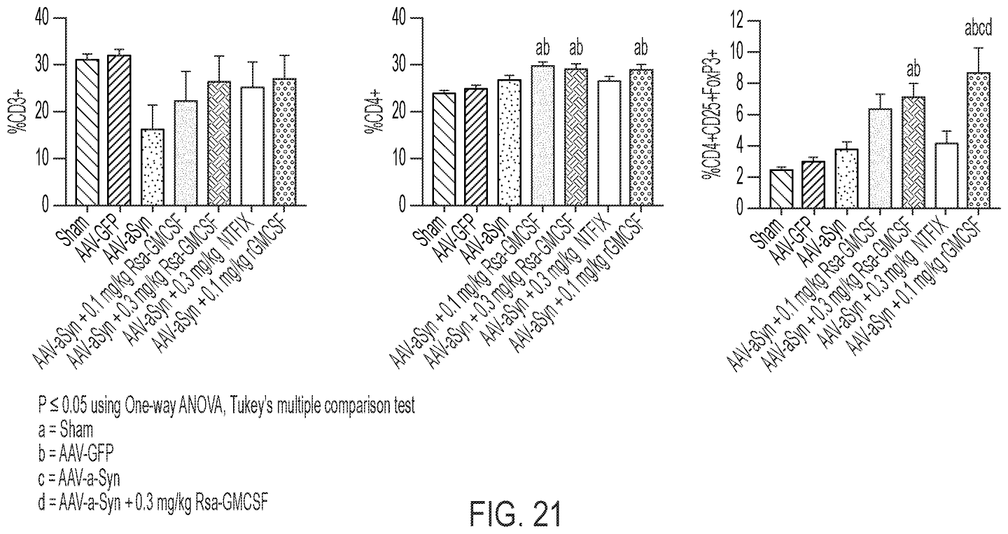

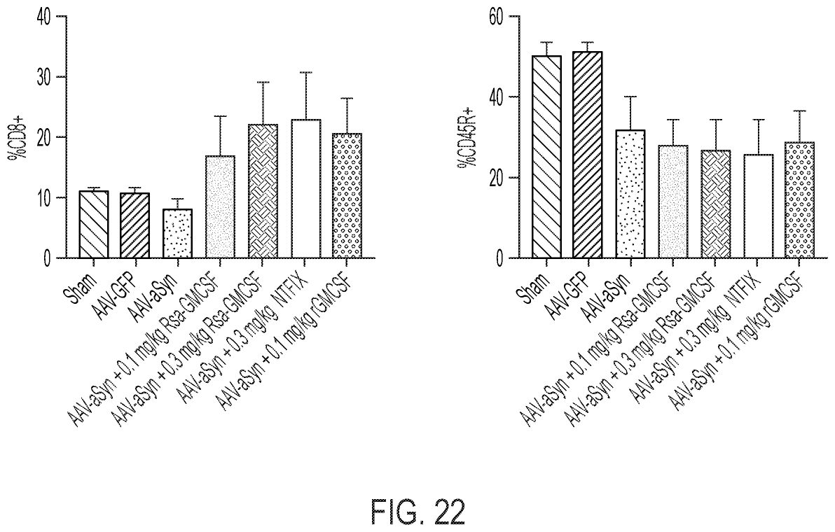

Flow cytometric analysis of splenic T cell populations indicated no significant change in CD3 percentage or CD4 percentage with all treatments; however, increases in CD4+CD25+FOXP3+ Treg percentages and CD8+ percentages were observed with both mRNA treatments, along with a slight decrease in CD45R+ percentages (FIGS. 21 and 22).

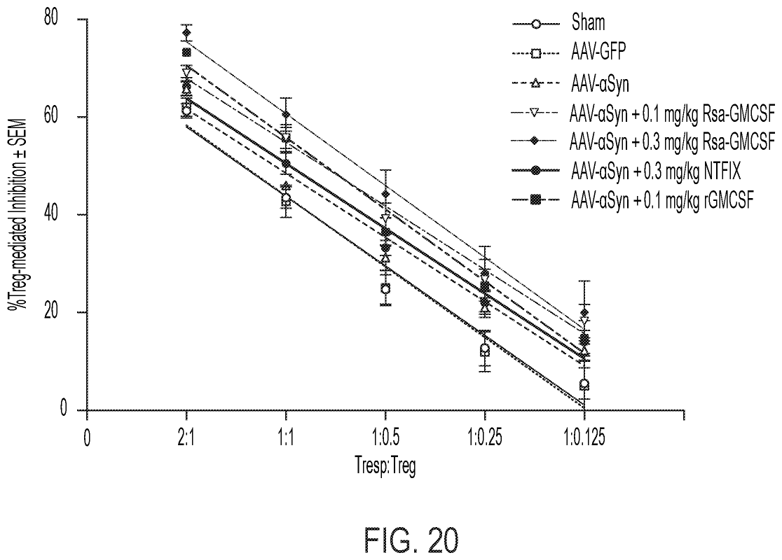

CD4+CD25+ Treg cells enriched from each treatment group were assessed for changes in their suppressive function to inhibit proliferation of CD4+CD25− Tresps (FIG. 20). Treatment with both dosages of LNP formulated Gm-csf mRNA resulted in enhanced suppressive function compared to PBS controls.

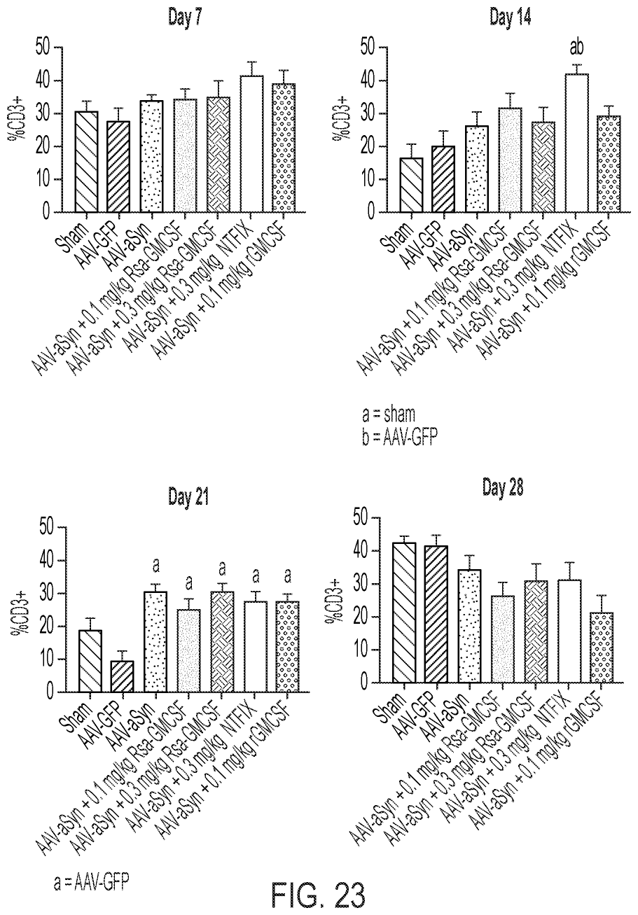

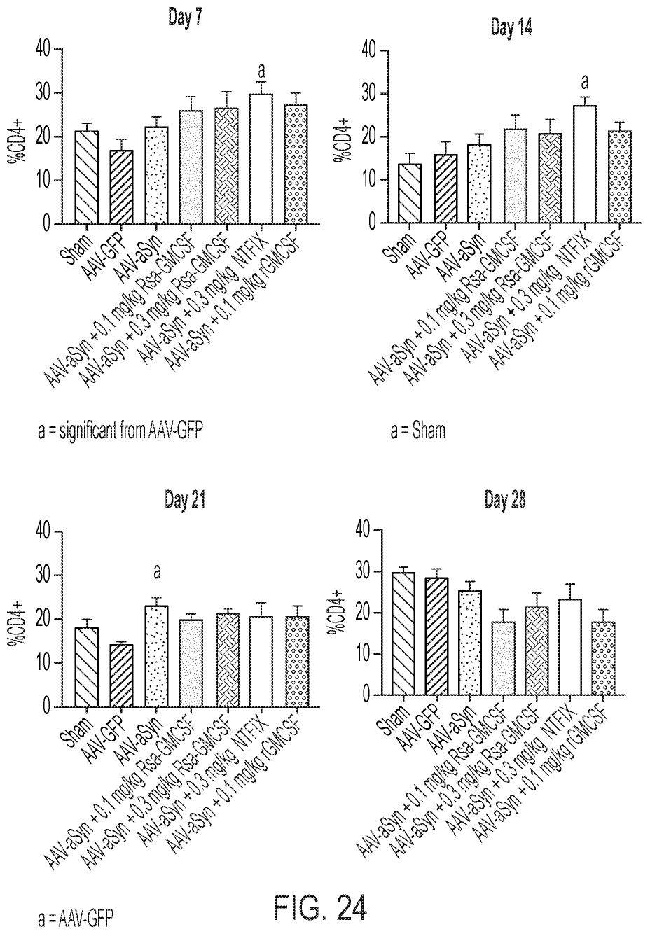

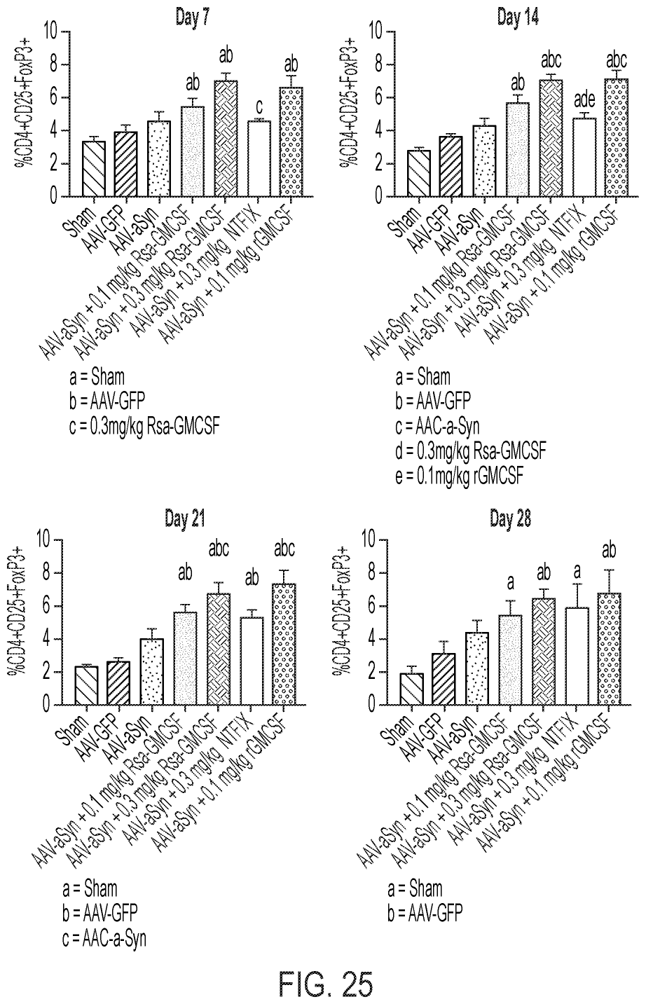

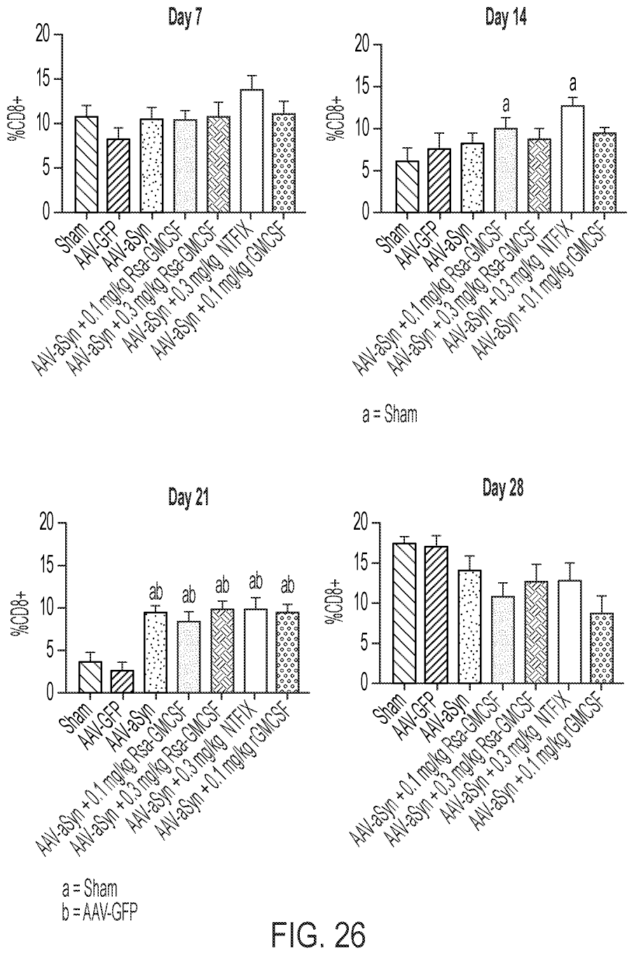

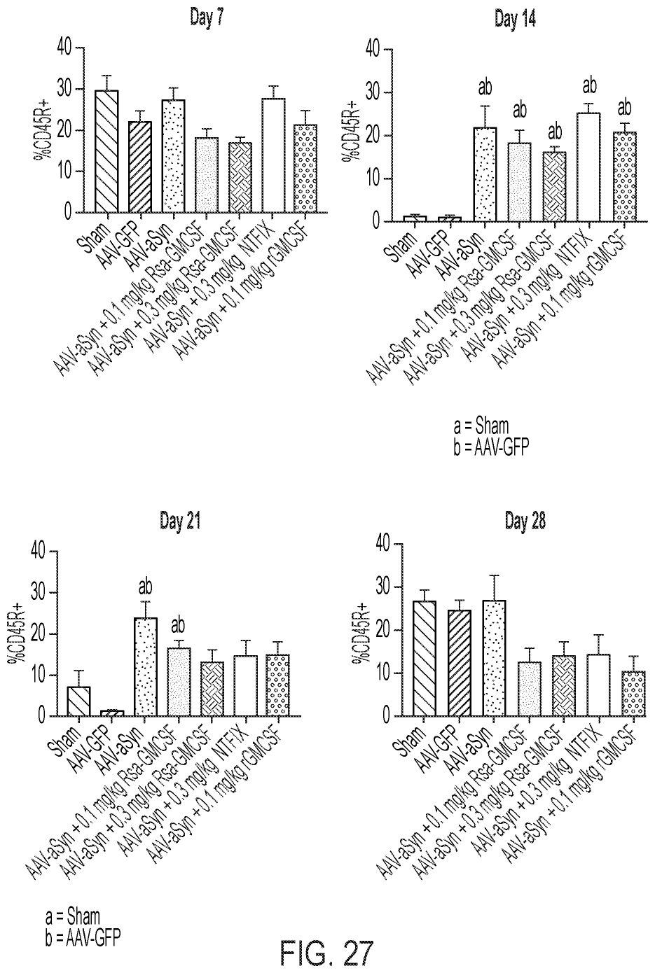

T cell levels were assessed in peripheral blood at days 7, 14, 21, and 28 (FIG. 23-27). Levels of CD3+, CD4+, CD4+CD25+FOXP3+CD8+, and CD45R+ cells were assessed. A mild increasing trend in CD4+CD25+FOXP3+ Treg frequencies was observed with LNP formulated RSA-conjugated Gm-csf mRNA treatment with increasing dosages (FIG. 25).

These results demonstrate that LNP formulated RSA-conjugated Gm-csfmRNA treatment in an alpha-syn model of Parkinson's disease resulted in increased Treg activities.

What is claimed is:

1. A lipid nanoparticle (LNP) composition comprising a polynucleotide encoding a human GM-CSF polypeptide for use, in the treatment of Parkinson's disease in a subject, wherein the GM-CSF polypeptide comprises the amino acid sequence of SEQ ID NO: 1, SEQ ID NO: 8, or SEQ ID NO: 187.

2. A method of treating Parkinson's disease in a subject, comprising administering to the subject an effective amount of a lipid nanoparticle (LNP) composition comprising a polynucleotide encoding a human GM-CSF polypeptide, wherein the GM-CSF polypeptide comprises the amino acid sequence of SEQ ID NO: 1, SEQ ID NO: 8, or SEQ ID NO: 187.

3. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 2.

4. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 3.

5. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 188.

6. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 216.

7. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 221.

8. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 219.

9. The LNP composition for use of claim 1, or the method of claim 2, wherein the polynucleotide encoding the human GM-CSF polypeptide comprises a nucleotide sequence having at least 85%, 90%, 95%, 96%, 97%, 98%, 99% or 100% identity to SEQ ID NO: 224.

10. The LNP composition for use, or the method of any one of claims 1-9, wherein the polynucleotide encoding the human GM-CSF polypeptide comprises at least one chemical modification.

11. The LNP composition for use, or the method of claim 10, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

12. The LNP composition for us, or the method of claim 11, wherein the chemical modification comprises N1-methylpseudouridine.

13. The LNP composition for use, or the method of any one of claims 1-12, wherein the LNP composition comprises: (i) an ionizable lipid, e.g., an amino lipid; (ii) a sterol or other structural lipid; (iii) a non-cationic helper lipid or phospholipid; and (iv) a PEG-lipid, e.g., a PEG-modified lipid.

14. The LNP composition for use, or the method of claim 13, wherein the ionizable lipid comprises Compound 18.

15. The LNP composition for use, or the method of claim 13, wherein the ionizable lipid comprises Compound 25.

16. The LNP composition for use or the method of claim 13, wherein the PEG-lipid is PEG DMG.

17. The LNP composition for use, or the method of any one of claims 1-16, wherein administration of LNP increases the level and/or activity of T regulatory cells in a sample (e.g., a sample from a subject), e.g., as determined by an assay in any one of Examples 2-8.

18. The LNP composition for use or the method of any one of claims 1-17, wherein administration of the LNP increases the level of T regulatory cells in a sample (e.g., a sample from a subject) by at least about 5%.

19. The LNP composition for use or the method of any one of claims 1-18, wherein the level of GM-CSF in tissues is not increased as compared to a reference, e.g., an appropriate control.

20. The LNP composition for use or the method of any one of claims 1-19, wherein nigrostriatal neurodegeneration and microglial activation is reduced as compared to a reference, e.g., an appropriate control.
